(19) United States
(12) Patent Application Publication
(10) Pub. No.: US 2026/0248860 A1
(43) Pub. Date: Aug. 27, 2026

(54) LIVER DISEASE REGULATORY FORMULATION AND USE THEREOF

(71) Applicant: SHANGHAI CELLIVER BIOTECHNOLOGY CO., LTD., Shanghai (CN)

(72) Inventors: Hongdan ZHANG, Shanghai (CN); Xuejing ZHU, Shanghai (CN); Renjie HUANG, Shanghai (CN)

(73) Assignee: SHANGHAI CELLIVER BIOTECHNOLOGY CO., LTD., Shanghai (CN)

(21) Appl. No.: 19/539,029

(22) Filed: Feb. 13, 2026

Related U.S. Application Data
(62) Division of application No. 18/463,267, filed on Sep. 7, 2023.
Application No. 18/463,267 is a continuation-in-part of application No. PCT/CN2022/079802, filed on Mar. 8, 2022.

(30) Foreign Application Priority Data
Mar. 8, 2021 (CN) 202110249543.7

(51) Int. Cl. A61K 35/407; A61K 31/7105; A61P 1/16; C12N 5/00; C12N 5/071

(52) U.S. Cl. CPC A61K 35/407; A61K 31/7105; A61P 1/16; C12N 5/0018; C12N 5/0672; C12N 2500/32; C12N 2500/38; C12N 2501/999; C12N 2502/14

(57) ABSTRACT

An objective of the present invention is to provide a liver disease regulatory formulation, which is beneficial to prevent the occurrence and development of a chronic liver disease by remodeling a liver regeneration microenvironment. The liver disease regulatory formulation, comprises a hepatocyte-derived liver progenitor cell or a secretory supernatant of the hepatocyte-derived liver progenitor cell.

### CROSS-REFERENCE TO RELATED APPLICATION

The present application is a divisional application of U.S. application Ser. No. 18/463,267, filed on Sep. 7, 2023. The U.S. application Ser. No. 18/463,267 is a continuation-in-part of international application of PCT application serial no. PCT/CN2022/079802, filed on Mar. 8, 2022, which claims the priority of a Chinese patent application No. 202110249543.7 filed on Mar. 8, 2021. The entirety of each of the above-mentioned patent applications is hereby incorporated by reference herein and made a part of this specification.

### BACKGROUND OF THE INVENTION

### 1. Technical Field

The present invention relates to the technical field of biotechnology, and in particular relates to an anti-hepatic fibrosis formulation, and a preparation method and use thereof.

### 2. Background Art

It has become an urgent clinical problem to be solved to find an effective method to promote liver regeneration and increase an effective liver volume and liver reserve, thereby improving the liver function condition of a patient, minimizing the incidence of serious complications related to liver cirrhosis and the like liver diseases, and creating conditions for subsequent effective treatment. The orderly regulation of a liver regeneration microenvironment is an important mechanism for maintaining a physiological function of a liver, and remodeling the liver regeneration microenvironment is of great significance to prevent the occurrence and development of chronic liver diseases.

Therefore, it is necessary to develop a novel liver disease regulatory formulation to solve the aforementioned problems existed in the prior art.

### SUMMARY OF THE INVENTION

An objective of the present invention is to provide a liver disease regulatory formulation, which is beneficial to prevent the occurrence and development of a chronic liver disease by remodeling a liver regeneration microenvironment.

The liver disease regulatory formulation of the present invention includes a hepatocyte-derived liver progenitor cell or a secretory supernatant of the hepatocyte-derived liver progenitor cell.

In some embodiments, the hepatocyte-derived liver progenitor cell is a liver precursor cell.

In some embodiments, the hepatocyte-derived liver progenitor cell is a liver precursor-like cell.

In some embodiments, the hepatocyte-derived liver progenitor cell is a human liver progenitor cell.

In some embodiments, the human hepatocyte-derived liver progenitor cell is a human liver progenitor cell.

In some embodiments, the human hepatocyte-derived liver progenitor cell is a human liver progenitor-like cell.

In some embodiments, the secretory supernatant of the hepatocyte-derived liver progenitor cell includes at least one miRNA which is at least one of miRNA-182, miRNA-183 and miRNA-574 and can effectively promote liver cell proliferation.

In some embodiments, the secretory supernatant of the hepatocyte-derived liver progenitor cell includes an active ingredient acting on a JAK-STAT pathway to inhibit activation of a hepatic stellate cell or induce death of the hepatic stellate cell.

In some embodiments, the secretory supernatant of the hepatocyte-derived liver progenitor cell includes at least one of at least one of a leukemia inhibitory factor, an endothelin, a colony stimulating factor, an amphiregulin and a fibroblast growth factor to achieve the effect of inhibiting activation of a hepatic stellate cell or inducing death of the hepatic stellate cell.

In some embodiments, the fibroblast growth factor is FGF19.

In some embodiments, the secretory supernatant of the hepatocyte-derived liver progenitor cell includes a secretory ingredient capable of inducing a receptor to establish effective immune tolerance by inhibiting proliferation of an immune cell.

In some embodiments, the immune cell is any one of a macrophage, a B cell, a T cell, an NK cell and an NKT cell.

In some embodiments, the liver disease regulatory formulation further includes a resuspending ingredient including at least one of physiological saline, a compound electrolyte solution, a buffer solution and a basal medium.

In some embodiments, the liver disease regulatory formulation further includes an auxiliary ingredient including at least one of immunosuppressive ingredient, serum, an antibiotic and a synergistic active ingredient.

In embodiments of the present invention, the secretory supernatant of the hepatocyte-derived liver progenitor cell is acquired from a culture product obtained by culturing the hepatocyte-derived liver progenitor cell in an in vitro medium.

In some embodiments, the in vitro medium is a basal medium which is at least one of a HepX medium, a DMEM/F12 cell culture medium, a William's E cell culture medium, a Neurobasal Medium cell culture medium, an MEM cell culture medium, a DMEM cell culture medium, a 1640RPMI cell culture medium and a F12 cell culture medium.

In some embodiments, the in vitro medium includes the basal medium and a serum-like substance.

In some embodiments, the in vitro medium consists of the basal medium and the serum-like substance. Based on the volume content of the basal medium, the content of the serum-like substance is no more than 20%.

In some embodiments, the in vitro medium is further added with a double antibody of which the content is no more than 2%.

In some embodiments, the in vitro medium further includes at least one of N2, B27, a growth factor, a ROCK kinase inhibitor, a Wnt signaling pathway agonist, a TGF-β signaling inhibitor, N-acetyl-L-cysteine and ascorbic acid.

In some embodiments, the hepatocyte-derived liver progenitor cell is obtained by culturing a primary hepatocyte in vitro with a hepatocyte expansion and transformation medium (TEM medium).

In some embodiments, the TEM medium includes the basal medium, the serum-free additive, the serum-like substance, the growth factor, the TGF-β signal inhibitor, the Wnt signaling pathway agonist, and the ROCK kinase inhibitor.

Based on the content in the basal medium, a content of the growth factor is 0.1-100 nanograms/milliliter, a content of the ROCK kinase inhibitor is 0.1-100 micromoles/liter, a content of the Wnt signaling pathway agonist is 0.1-50 micromoles/liter, a content of the TGF-β signal inhibitor is 0.1-100 micromoles/liter, a content of the serum-like substance is no more than 20%, and a volume content of the serum-free additive is no more than 2%.

In some embodiments, the TEM medium further includes at least one of N-acetyl-L-cysteine and ascorbic acid.

In some embodiments, the growth factor is at least one of an epidermal growth factor, a fibroblast growth factor, a vascular endothelial growth factor, a platelet-derived growth factor, a hepatocyte growth factor, an interleukin-6 and oncostatin.

In some embodiments, the ROCK kinase inhibitor is at least one of Fasudil, Y-27632, Thiazovivin and SB-772077-B.

In some embodiments, the Wnt signaling pathway agonist is at least one of a recombinant Wnt protein, a recombinant R-spondin protein and a glycogen synthase kinase 3β inhibitor.

In some embodiments, the TGF-β signal inhibitor is at least one of RepSox, SB431542 and A83-01.

In some embodiments, the serum-like substance in any one of the in vitro medium and the TEM medium is animal-derived serum.

In some embodiments, the animal-derived serum in any one of the in vitro medium and the TEM medium can be replaced with a serum substitute.

In some embodiments, the serum substitute is an animal-derived ingredient-free platelet and a derivative thereof.

In some embodiments, the serum substitute is sphingosine monophosphate and indoleacetic acid.

In some embodiments, the animal-derived serum is fetal bovine serum.

In the TEM medium of some embodiments, based on the content in the basal medium, a content of the sodium pyruvate is 0.5-1.5 millimoles/liter, a content of the ascorbic acid is 5-50 micrograms/milliliter, a content of the epidermal growth factor is 5-25 nanograms/milliliter, a content of the hepatocyte growth factor is 5-25 nanograms/milliliter, a content of the ROCK kinase inhibitor is 5-20 micromoles/liter, a content of the Wnt signaling pathway agonist is 1-5 micromoles/liter, a content of the TGF-β signal inhibitor is 0.5-2 micromoles/liter, a content of the sphingosine monophosphate is 0.5-2 micromoles/liter, a content of the indoleacetic acid is 2-10 micromoles/liter, a volume percentage of the N2 additive and the B27 additive is no more than 1%, and a content of N-acetyl-L-cysteine is 0.5-10 micromoles/liter.

An embodiment of the present invention provides in vitro use of the liver disease regulatory formulation, including co-culturing the liver disease regulatory formulation with a target cell. The target cell is any one of a primary hepatocyte, a hepatic stellate cell, a macrophage and an immune-related cell.

In some embodiments, the step of co-culturing the liver disease regulatory formulation with the target cell includes co-culturing the liver disease regulatory formulation with the hepatic stellate cell by using a co-culture medium, and the content of the liver disease regulatory formulation is no less than 1% based on a volume percentage of the co-culture medium.

During the co-culture process of some embodiments, a plating density of the target cell is 1×10⁴ cells/square centimeter.

In some embodiments, the co-culture medium consists of the basal medium and the serum-like substance.

In some embodiments, in the co-culture medium, based on the volume percentage of the basal medium, the content of the serum-like substance is no more than 20%.

In some embodiments, the co-culture medium further includes an activator of the hepatic stellate cell.

In some embodiments, the activator of the hepatic stellate cell is a hepatic stellate cell activating factor.

In some embodiments, the hepatic stellate cell activating factor is TGF-β1.

In some embodiments, the step of co-culturing the liver disease regulatory formulation with the target cell includes co-culturing the liver disease regulatory formulation with a hepatic macrophage model which is an inflammatory cell model or a repair cell model.

In some embodiments, the step of co-culturing the liver disease regulatory formulation with the target cell includes co-culturing the liver disease regulatory formulation with the immune-related cell, and inducing proliferation of the immune-related cell by using a stimulator.

In some embodiments, the immune-related cell is any one of a peripheral blood mononuclear cell and a spleen cell.

In some embodiments, the step of co-culturing the liver disease regulatory formulation with the immune-related cell includes resuspending the liver disease regulatory formulation by using the co-culture medium, and controlling a volume concentration of the liver disease regulatory formulation in the co-culture medium to be no less than 5%, so that the of the liver disease regulatory formulation on the proliferation of the immune-related cell is no less than 30%.

In some embodiments, the step of co-culturing the liver disease regulatory formulation with the immune-related cell includes co-culturing different liver disease regulatory formulations with the immune-related cell, and a culture supernatant of the contained in the different liver disease regulatory formulations is derived from different donors.

The use of the liver disease regulatory formulation of the embodiments of the present invention in treatment of a liver disease includes investigating an effect on liver regeneration after an in vivo animal model is intervened with the liver disease regulatory formulation.

In some embodiments, the in vivo animal model is any one of a carbon tetrachloride-induced mouse acute liver failure model, an acetaminophen-induced mouse acute liver failure model, a thioacetamide-induced mammalian liver cirrhosis model, a carbon tetrachloride-induced mammalian liver cirrhosis model, a mammalian nonalcoholic steatohepatitis model, a mouse autoimmune hepatitis model induced by ConA and mediated by a T cell and a NKT cell, a rat model of immune rejection after liver cell or liver tissue transplantation, and a post-liver transplantation acute host anti-graft reaction model.

### BRIEF DESCRIPTION OF THE DRAWINGS

FIG. 1 is a transmission electron micrograph of a PHH Exo sample of Example 1-1;

FIG. 2 is a transmission electron micrograph of a Hep Exo sample of Example 1-1;

FIG. 3 is a comparison chart of the average particle size of exosomes in a PHH Exo sample and a Hep Exo sample of Example 1-1;

FIG. 4 is a comparison chart of the expression of CD63 and CD81 in exosomes from different cell sources as detected by flow cytometry in Example 1-1;

FIG. 5 is a photograph of the expression of CD63 and TSG101 in different cells and exosomes from different cell sources as investigated by a western blotting test in Example 1-1;

FIG. 6 is a photograph of immunofluorescence confocal detection results of the PHH Exo sample and the Hep Exo sample of Example 1-2 after labeling, staining and co-culture;

FIG. 7 is a comparison chart of BrdU incorporation of cells obtained after co-culture of the PHH Exo sample and the Hep Exo sample with different exosome concentrations in Example 1-3;

FIG. 8 is an immunofluorescence photograph obtained after EdU fluorescent staining of cells obtained after co-culturing the PHH Exo sample and the Hep Exo sample with PHHs respectively in Example 1-3;

FIG. 9 is an immunofluorescence photograph obtained by Ki67 immunofluorescence staining of cells obtained after co-culturing the PHH Exo sample and the Hep Exo sample with PHHs respectively in Example 1-3;

FIG. 10 is a comparison chart of flow cytometry results obtained by flow cell-cytometry cycle analysis of a PHH Exo-cell and a Hep Exo-cell in Example 1-4;

FIG. 11 is a comparison chart of miRNA expression levels of cell cycle-related molecules obtained by performing real-time fluorescent quantitative PCR analysis on a control group, the PHH Exo-cell and the Hep Exo-cell in Example 1-4;

FIG. 12 is a comparison chart of imprint photographs obtained by investigating the expression of cell cycle-related molecules in the control group, the PHH Exo-cell and the Hep Exo-cell using the western blotting test in Example 1-4;

FIG. 13 is a clustering diagram of miRNA sequencing expression of exosomes from different cell sources in Example 1-5;

FIG. 14 is a comparison chart of BrdU incorporation in each transfected cell as detected by ELISA 48 hours after in vitro transfection of hsa-miR-182, hsa-miR-183, hsa-miR-149, hsa-miR-215, hsa-miR-574, hsa-miR-654 and hsa-miR-675 with primary hepatocytes in Example 1-5;

FIG. 15 is EdU fluorescence micrographs obtained after EdU staining respectively 48 hours after transfection of hsa-miR-182, hsa-miR-183 and hsa-miR-574 with primary hepatocytes in Example 1-5;

FIG. 16 shows the comparison of EdU incorporation ratio of each transfected cell as detected by an EdU fluorescence method 48 hours after hsa-miR-182, hsa-miR-183 and hsa-miR-574 are respectively transfected with primary hepatocytes in Example 1-5.

FIG. 17 shows the change of a survival rate over time after the modeling of each mouse in a Hep Exo treatment group and a PBS control group is completed in Example 1-6;

FIG. 18 is HE-stained photographs of liver tissue sections 24 hours and 48 hours after modeling of mice in a normal control group, the PBS control group and the Hep Exo treatment group in Example 1-6;

FIG. 19 is a comparison chart of immunohistochemical Ki67 staining of liver tissue sections of mice in the PBS control group and the Hep Exo treatment group in Example 1-6;

FIG. 20 is a comparison chart of the percentage of Ki67 positive cells in paraffin sections of liver tissues of each mouse in the normal group, the PBS control group and the Hep Exo treatment group in Example 1-6;

FIG. 21 shows the comparison results of ALT levels of each mouse in the normal group, the PBS control group and the Hep Exo treatment group at different times in Example 1-6;

FIG. 22 shows the comparison results of AST levels of each mouse in the normal group, the PBS control group and the Hep Exo treatment group at different times in Example 1-6;

FIG. 23 shows the change of a survival rate over time of mice in each group of a CCl4 model in Example 1-7;

FIG. 24 shows the change of a survival rate over time of mice in each group of a APAP model in Example 1-7;

FIG. 25 is a comparison chart of AST levels at different times after modeling of each mouse of the CCl4 model in Example 1-8;

FIG. 26 is a comparison chart of ALT levels at different times after modeling of each mouse of the CCl4 model in Example 1-8;

FIG. 27 is a comparison chart of AST levels and ALT levels 24 hours after modeling of each mouse of the APAP model in Example 1-8;

FIG. 28 is a comparison chart of pathological tissue sections at different times after modeling of each mouse of the CCl4 model in Example 1-8;

FIG. 29 is a comparison chart of pathological tissue sections 24 hours after modeling of each mouse of the APAP model in Example 1-8;

FIG. 30 is a comparison chart of immunohistochemical Ki67 staining of liver tissue sections at different times after modeling of the mice of a CCl4-NC agomir group and the mice of a CCl4-miRNA 183-5p agomir group is completed in Example 1-8;

FIG. 31 is a comparison chart of immunohistochemical Ki67 staining of liver tissue sections 24 hours after modeling of the mice of an APAP-NC agomir group and the mice of an APAP-miRNA 183-5p agomir group is completed in Example 1-8;

FIG. 32 is a schematic diagram showing the comparison of the percentage of Ki67 positive cells in the liver tissue sections of the mice of the CCl4-NC agomir group and the mice of the CCl4-miRNA 183-5p agomir group at different times after the modeling is completed in Example 1-8;

FIG. 33 is a comparison chart of the expression of cell cycle-related proteins in liver tissues at different times after modeling of the mice of the CCl4-miRNA 183-5p agomir group in Example 1-8.

FIG. 34 is a schematic diagram showing the gene expression of human primary hepatocytes in Example 2-1;

FIG. 35 is a schematic diagram showing the gene expression of human liver precursor-like cells in Example 2-1;

FIG. 36 is a comparison photograph of the microscopic morphology of each hepatic stellate cell in an experimental group, a control group and a group activated with TGF-β1 in Example 2-2;

FIG. 37 is a comparison chart of relative mRNA expression of genes related to activation of HSCs in the experimental group, the control group and the group activated with TGF-β1 in Example 2-2;

FIG. 38 is a photograph of the microscopic morphology of the cell aggregates of the experimental group observed under a transmission microscope in Example 2-2;

FIG. 39 shows the analysis result obtained by performing flow cytometry assay on the cell aggregates of the control group in Example 2-2;

FIG. 40 shows the analysis result obtained by performing flow cytometry assay on the cell aggregates of the experimental group in Example 2-2;

FIG. 41 is a comparison chart of the expression of fibrosis-related proteins and the expression of key fibrosis signals in each group of cells obtained by western blot analysis of the cell aggregates of the control group, the group activated with TGF-β1 and the experimental group in Example 2-2;

FIG. 42 is a comparison chart of the expression of liver progenitor cell genes and liver parenchymal cell markers of murine primary hepatocytes and murine liver precursor-like cells in Example 2-3;

FIG. 43 is a comparison chart of photographs of the microscopic morphology of HSCs-T6 cells in each group after co-culture of the control group, the group activated with TGF-β1 and the experimental group for 48 hours in Example 2-4;

FIG. 44 is a comparison chart of the relative mRNA expression of genes related to activation of HSCs in the HSCs-T6 of the control group, the group activated with TGF-β1 and the experimental group in Example 2-4;

FIG. 45 is a photograph of the microscopic morphology of cells of the experimental group in Example 2-4;

FIG. 46 shows the analysis result obtained by performing flow cytometry assay on the cells of the group activated with TGF-β1 in Example 2-4;

FIG. 47 shows the analysis result obtained by performing flow cytometry assay on the cells of the experimental group in Example 2-4;

FIG. 48 is a schematic diagram of a visualized network between a JAK-STAT pathway and proteins involved in growth factor activity, cytokine activity and receptor-ligand activity in a first anti-hepatic fibrosis formulation, in the first anti-hepatic fibrosis formulation of Example 2-5;

FIG. 49 is a comparison chart of the expression of a pSTAT1 signal in each group of cells shown in FIG. 16 obtained after western blot analysis of the control group, the group activated with TGF-β1 and the experimental group in Example 2-6;

FIG. 50 is a comparison chart of the analysis results of the flow cytometry of the cell aggregates of each one of the control group, the group activated with TGF-β1 and the experimental group in Example 2-7;

FIG. 51 is a comparison chart of the percentage of apoptotic cells in each group obtained by statistics according to FIG. 50;

FIG. 52 is a comparison chart of photographs of the microscopic morphology of liver tissue sections of mice in a normal group, a group injected with PBS and a group intervened with an anti-hepatic fibrosis formulation in Example 2-8;

FIG. 53 is a comparison photograph of the distribution state of activated hepatic stellate cells in the liver of the mice in the normal group, the group injected with PBS and the group intervened with an anti-hepatic fibrosis formulation in Example 2-8.

FIG. 54 is a comparison chart of the expressions of collagen-related genes and fibrosis-related genes in LX-2 cells obtained after co-culture of a LX-2 group, a group added with LX-2 and a co-culture group for 48 hours in Example 2-9;

FIG. 55 is a comparison chart of photographs obtained after H&E staining, picro-Sirius sirus red staining, Masson trichrome staining and fibronectin (FN) immunostaining of liver tissues taken from the normal group, the sham operation group and the cell transplantation group in Example 2-10;

FIG. 56 is a comparison chart of the relative quantification of liver fibrosis regions and fibronectin positive-stained regions from statistics after picro-Sirius sirus red staining, Masson trichrome staining and fibronectin (FN) immunostaining of liver tissues taken from the normal group, the sham operation group and the cell transplantation group in Example 2-10;

FIG. 57 shows the analysis results of the determination of hydroxyproline content in a normal group, a sham operation group and a cell transplantation group in Example 2-10;

FIG. 58 shows the analysis results of liver fibrosis scoring of liver tissues taken from the sham operation group and the cell transplantation group in Example 2-10;

FIG. 59 is a comparison chart of photographs obtained by Ki67 immunohistochemical staining of liver tissues taken from the normal group, the sham operation group and the cell transplantation group in Example 2-10;

FIG. 60 shows the quantification result of positive-stained cells from statistics according to the photographs shown in FIG. 59;

FIG. 61 is a comparison chart of photographs obtained after H&E staining, picro-Sirius sirus red staining, Masson trichrome staining and fibronectin (FN) immunostaining of liver tissues taken from the normal group, the sham operation group and the cell transplantation group in Example 2-11;

FIG. 62 is a comparison chart of the relative quantification of liver fibrosis regions and fibronectin positive-stained regions from statistics after picro-Sirius sirus red staining, Masson trichrome staining and fibronectin (FN) immunostaining of liver tissues taken from the normal group, the sham operation group and the cell transplantation group in Example 2-11;

FIG. 63 shows the analysis results of the determination of hydroxyproline content in the liver tissues taken from the normal group, the sham operation group and the cell transplantation group in Example 2-11;

FIG. 64 shows the analysis results of liver fibrosis scoring of liver tissues taken from the sham operation group and the cell transplantation group in Example 2-11;

FIG. 65 is a heat map obtained after analyzing the gene expression levels related to the occurrence of liver fibrosis related to fibrosis promoting, extracellular matrix, and signal transduction of blood in the normal group, the sham operation group and the cell transplantation group in Example 2-11;

FIG. 66 is a bright-field photograph of HepLPCs obtained by bright-field photography of the cells obtained after being cultured in a TEM medium for 10 days in Example 3-1;

FIG. 67 shows the analysis result of the proportion of total macrophages obtained by cell identification of BMDMs induced by mouse GM-CSF with flow cytometry in Example 3-2;

FIG. 68 shows the analysis result of the proportion of type M1 macrophages obtained by cell identification of BMDMs induced by LPS in vitro orientational polarization with flow cytometry in Example 3-2;

FIG. 69 is a comparison chart of the expression of M1-related inflammatory genes obtained by cell identification of a Control group and a DM+LPS group by flow cytometry in Example 3-2;

FIG. 70 is a comparison chart of the expression of M1-related inflammatory genes in each group obtained after RNA extraction and gene expression analysis of a HepLPCs-CM+LPS group, the Control group and the DM+LPS group in Example 3-2;

FIG. 71 is a comparison chart of the expression levels of M1-related inflammatory genes obtained by detecting the concentration of cytokines in the cell culture supernatants of the HepLPCs-CM+LPS group, the Control group and the DM+LPS group in Example 3-2;

FIG. 72 shows the analysis result of the proportion of type M2 macrophages obtained by cell identification of BMDMs induced by IL-4 in vitro orientational polarization with flow cytometry in Example 3-3;

FIG. 73 is a comparison chart of the expression of M2-related inflammatory genes obtained by cell identification of the Control group and the DM+IL-4 group by flow cytometry in Example 3-3;

FIG. 74 is a comparison chart of the secretion of a M2-related inflammatory factor IL10 in each group obtained by RNA extraction and gene expression analysis of the HepLPCs-CM+IL-4 group, the Control group and the DM+IL-4 group in Example 3-3;

FIG. 75 is a DAPI nucleostained fusion diagram after interaction of different concentrations of exosomes with macrophage BMDMs for 6 hours in Example 3-4;

FIG. 76 is a comparison chart of the expression of M1-related inflammatory genes in the Control group, the DM+LPS group, the CM+LPS group and the EV+LPS group in Example 3-4;

FIG. 77 is a comparison chart of the expression of M2-related inflammatory genes in the Control group, the DM+IL4 group, the CM+IL4 group and the EV+IL4 group in Example 3-5;

FIG. 78 is a stained photograph obtained after paraffin embedding, sample preparation by sectioning and H&E staining of the liver tissues of an early NASH mouse model of Example 3-6;

FIG. 79 is a stained photograph obtained after paraffin embedding, sample preparation by sectioning and H&E staining of the liver tissues of mice of the control group of Example 3-6;

FIG. 80 is a stained photograph obtained through Masson staining after paraffin embedding and sample preparation by sectioning of the liver tissues of the early NASH mouse model in Example 3-6;

FIG. 81 is a stained photograph obtained through oil red O staining after paraffin embedding and sample preparation by sectioning of the liver tissues of the early NASH mouse model in Example 3-6;

FIG. 82 is a stained photograph obtained from immunohistochemical staining of a type M1 macrophage-specific marker CD68 after paraffin embedding and sample preparation by sectioning of the liver tissues of the early NASH mouse model in Example 3-6;

FIG. 83 is a stained photograph obtained from immunohistochemical staining of a type M2 macrophage-specific marker CD163 after paraffin embedding and sample preparation by sectioning of the liver tissues of the early NASH mouse model in Example 3-6;

FIG. 84 is a comparison chart of blood biochemistry and TC and TG contents between the early NASH mouse model and the control group in Example 3-6;

FIG. 85 is a comparison chart of H&E stained photographs and oil red O stained photographs of the livers of mice in the sham operation group, a group treated with a low dose of cells and a group treated with a high dose of cells in Example 3-7;

FIG. 86 is a comparison chart of the NAS scores of the sham operation group, the group treated with a low dose of cells and the group treated with a high dose of cells in Example 3-7;

FIG. 87 is a Masson stained photograph of the liver sections of the mice in the sham operation group, the group treated with a low dose of cells and the group treated with a high dose of cells in Example 3-7;

FIG. 88 is a comparison chart of the statistical results of liver fibrosis regions in the stained photograph of each group of FIG. 11 in Example 3-7;

FIG. 89 is a stained photograph obtained by Ki67 staining of the liver sections of the mice in the sham operation group, the group treated with a low dose of cells and the group treated with a high dose of cells in Example 3-7;

FIG. 90 is a comparison chart of the levels of indicators ALT, AST and LDH in the blood of mice in a normal control group, an immunosuppressive group, a model group administrated orally with physiological saline, the sham operation group, the group treated with a low dose of cells and the group treated with a high dose of cells in Example 3-7;

FIG. 91 is a comparison chart of TC and TG contents in the livers of the mice in the normal control group, the sham operation group, the group treated with a low dose of cells and the group treated with a high dose of cells in Example 3-7;

FIG. 92 is a comparison chart of stained photographs obtained after anti-CD163 and anti-CD68 immunohistochemical staining of the mice in the sham operation group, the group treated with a low dose of cells and the group treated with a high dose of cells in Example 3-7;

FIG. 93 is a comparison chart of the numbers of CD163⁺ macrophages and CD68⁺ macrophages in the livers of each group obtained by statistics according to FIG. 92;

FIG. 94 is a comparison chart of H&E stained photographs of liver sections of the mice in a sham operation group, a group treated with a low dose of cells and a group treated with a high dose of cells of a middle-advanced NASH mouse model in Example 3-8;

FIG. 95 is a comparison chart of Masson stained photographs of liver sections of the mice in a sham operation group, a group treated with a low dose of cells and a group treated with a high dose of cells of a middle-advanced NASH mouse model in Example 3-8;

FIG. 96 is a comparison chart of the statistical results of liver fibrosis regions in each group obtained by statistics according to FIG. 96;

FIG. 97 shows the analysis result of the proportion of total macrophages obtained by cell identification of BMDMs induced by mouse GM-CSF with flow cytometry in Example 3-9;

FIG. 98 shows the analysis result of the proportion of type M1 macrophages obtained by cell identification of BMDMs induced by LPS in vitro orientational polarization with flow cytometry in Example 3-9;

FIG. 99 is a comparison chart of the expression of M1-related inflammatory genes obtained by cell identification of a Control group and a DM+LPS group by flow cytometry in Example 3-9;

FIG. 100 shows the analysis result of the proportion of type M2 macrophages obtained by cell identification of BMDMs induced by IL-4 in vitro orientational polarization with flow cytometry in Example 3-10;

FIG. 101 is a comparison chart of the expression of M2-related inflammatory genes obtained by cell identification of the Control group and the DM+IL-4 group by flow cytometry in Example 3-10;

FIG. 102 is a comparison chart of the expression of M1-related inflammatory genes in the Control group, the DM+LPS group, the CM+LPS group and the EV+LPS group in Example 3-11;

FIG. 103 is a comparison chart of the expression of M2-related inflammatory genes in the Control group, the DM+IL4 group, the CM+IL4 group and the EV+IL4 group in Example 3-12;

FIG. 104 is a comparison chart of the expression of various liver precursor-related markers in HepLPCs cells in Example 4-1;

FIG. 105 is a bright-field photograph of HepLPCs obtained by bright-field photography of the cells obtained after being cultured in a TEM medium for 10 days in Example 4-1;

FIG. 106 is a comparison chart obtained by investigating the proliferation inhibition of spleen cells by HepLPC-CM in a positive control group and each co-culture group with flow cytometry in Example 4-1;

FIG. 107 is a comparison chart obtained by investigating the proliferation inhibition of spleen cells by HepLPC-CM derived from a donor 1 and the proliferation of spleen cells in a corresponding positive control group with flow cytometry in Example 4-2;

FIG. 108 is a comparison chart obtained by investigating the proliferation inhibition of spleen cells by HepLPC-CM derived from a donor 2 and the proliferation of spleen cells in a corresponding positive control group with flow cytometry in Example 4-2;

FIG. 109 is a comparison chart obtained by investigating the proliferation inhibition of spleen cells by HepLPC-CM derived from a donor 3 and the proliferation of spleen cells in a corresponding positive control group with flow cytometry in Example 4-2;

FIG. 110 is a comparison chart obtained by investigating the proliferation inhibition of spleen cells by HepLPC-CM derived from a donor 4 and the proliferation of spleen cells in a corresponding positive control group with flow cytometry in Example 4-2;

FIG. 111 is a comparison chart obtained by investigating the proliferation inhibition of spleen cells by HepLPC in the positive control group, a FK506 control group and each co-culture group with flow cytometry in Example 4-3;

FIG. 112 is a comparison chart obtained by investigating the proliferation inhibition of spleen cells by HepLPC derived from a donor 1 and the proliferation of spleen cells in a corresponding positive control group with flow cytometry in Example 4-4;

FIG. 113 is a comparison chart obtained by investigating the proliferation inhibition of spleen cells by HepLPC derived from a donor 2 and the proliferation of spleen cells in a corresponding positive control group with flow cytometry in Example 4-4;

FIG. 114 is a comparison chart obtained by investigating the proliferation inhibition of spleen cells by HepLPC derived from a donor 3 and the proliferation of spleen cells in a corresponding positive control group with flow cytometry in Example 4-4;

FIG. 115 is a comparison chart obtained by investigating the proliferation inhibition of spleen cells by HepLPC derived from a donor 4 and the proliferation of spleen cells in a corresponding positive control group with flow cytometry in Example 4-4;

FIG. 116 is a comparison chart obtained by investigating the proliferation inhibition of PBMCs in a group co-cultured with PBMCs by HepLPC derived from each donor and the proliferation of PBMCs in a corresponding positive control group with flow cytometry in Example 4-5;

FIG. 117 shows a curve of the relationship between different ConA injection doses and mouse survival rate in Example 4-6;

FIG. 118 is a comparison chart of the levels of indicators ALT, AST and LDH in each group obtained by taking blood from the orbits of the mice in a group injected with a dose of 8 milligrams/kilogram at different times and investigating through analysis of a blood biochemical level in Example 4-7;

FIG. 119 is a comparison chart of the levels of indicators ALT, AST, LDH and ALP in each group obtained by taking blood from the orbits 6 hours after the injection of each mouse in the experimental group 1 and the control group is completed and investigating through analysis of a blood biochemical level in Example 4-7; and

FIG. 120 is a comparison chart of the levels of indicators ALT, AST and LDH in each group obtained by taking blood from the orbits 6 hours after the injection of each mouse in the experimental group 2 and the control group is completed and investigating through analysis of a blood biochemical level in Example 4.

### DETAILED DESCRIPTION OF THE EMBODIMENTS

In order to make the objectives, technical solutions and advantages of the embodiments of the present invention clearer, the following will clearly and completely describe the technical solutions in the embodiments of the present invention, and the described embodiments are a part rather than all of the embodiments of the present invention. All other embodiments obtained by a person of ordinary skill in the art based on the embodiments of the present invention without creative efforts are fall within the protection scope of the present invention. The technical or scientific terms used herein shall have the usual meanings understood by those of ordinary skill in the art to which the present invention belongs, unless otherwise defined. The “comprise(s)/comprising” or “include(s)/including” and the like similar words as used herein mean that the elements or articles appearing before the word encompass the elements or articles listed after the word and their equivalents, and do not exclude other elements or articles.

In each embodiment of the present invention, cell culture is carried out in a cell incubator under an environment of 37 degrees Celsius and a carbon dioxide concentration of 5%, unless otherwise specified. The media used for cell culture and various reagents used for cell processing, e.g. a buffer, are sterilized and filtered with a 0.22-micron filter before use to remove impurities.

For the data related to statistical analysis in each example of the present invention, the experiment of each group is repeated for at least 3 times, and the data of the experimental results are statistically analyzed by using GraphPad Prism 8.0 software. A two-tailed unpaired t-test is used for calculating a statistical difference in terms of comparison between two sets of data, and ANOVA analysis of variance is used for calculating a statistical difference in terms of comparison among multiple sets of data. p<0.05 is considered to be statistically different, and in the accompanying drawings: * represents P<0.05; ** represents P<0.005; *** represents P<0.001; and **** represents P<0.0001.

The following is explained in detail with particular examples.

The following examples prepare a hepatocyte regulatory formulation including at least one miRNA as a liver disease regulatory formulation, and use of this liver disease regulatory formulation is investigated. The details were as follows:

### Examples 1-1

In this example, human primary hepatocytes (abbreviated as PHHs) and human liver precursor-like cells (abbreviated as HepLPCs) were used as seed cells. They were cultured with a medium containing a serum-like substance and a medium free of the serum-like substance, and then successfully separated to obtain an exosome that had a particle size of about 100 nanometers and expressed exosome marker proteins TSG101, CD63 and CD81.

The PHHs of this example were purchased from Liver Biotechnology (Shenzhen) Co., Ltd, Guangzhou under a batch number of Lot #201904001; the HepLPCs were available from Celliver Biotechnology Co., Ltd. under a batch number of XLV-19006; a Hep-X basal medium was available from Shanghai Basalmedia Technologies Co., Ltd.; fetal bovine serum, a 1% penicillin-streptomycin solution and murine tail collagen were all available from Gibco; a hepatocyte growth factor HGF was available from Novoprotein; an epithelial cell growth factor EGF was available from Novoprotein; a ROCK kinase inhibitor Y-27632 was available from Topscience; a Wnt signaling pathway agonist CHIR-99021 was available from Topscience; a TGF-β signal inhibitor A-8301 was available from Topscience; CD63-FITC and CD81-PE flow cytometry antibodies were all from BD bioscience, USA.

The composition of the medium containing a serum-like substance as used was as follows: a Hep-X basal medium, as well as a N2 nutritional supplement (100×) with a content of 1%, a B27 nutritional supplement (50×) with a content of 1%, 10% fetal bovine serum FBS, a penicillin-streptomycin solution with a content of 1%; a hepatocyte growth factor HGF with a content of 20 ng/mL, an epithelial growth factor EGF with a content of 50 ng/mL, a ROCK kinase inhibitor Y-27632 with a content of 10 μM, a Wnt signaling pathway agonist CHIR-99021 with a content of 3 μM, and a TGF-β signal inhibitor A-8301 with a content of 1 μM, based on the volume in the Hep-X basal medium.

The composition of the medium free of the serum-like substance as used was the constituent components of the medium containing a serum-like substance after removal of the fetal bovine serum.

Both the medium containing the serum-like substance and the medium free of the serum-like substance were filtered with a 0.22-micron filter before use to remove impurities.

This example provided a process of acquiring precipitated substances containing exosomes respectively from two kinds of seed cells, specifically:
the seed cells were inoculated into a 15 cm culture dish at an inoculation density of 1×10⁵ cells/square centimeter, and added with 2 ml of the medium containing a serum-like substance each well to culture until the cell confluence was no less than 95% and the growth state was good, thereby completing the expansion culture. During the process of the expansion culture, the medium containing a serum-like substance was replaced every 2-3 days.

After the expansion culture was completed, the culture medium in the 15 cm culture dish was replaced with the medium free of the serum-like substance, the culture was continued for 48 hours, and then the culture supernatant was collected. The exosome isolation kit ExoQuick TC® ULTRA EV Isolation available from System Biosciences, USA was used for separating a precipitated substance derived from PHHs and a precipitated substance derived from HepLPCs from the culture supernatant. The specific operation steps were stated in the instructions attached to the exosome isolation kit, and would not be repeated here.

In this example, the aforementioned two precipitated substances were analyzed by utilizing transmission electron microscopy, nanoparticle tracking detection and flow cytometry. The precipitated substance derived from PHHs was abbreviated as a PHH Exo sample, and the precipitated substance derived from HepLPCs was abbreviated as a Hep Exo sample.

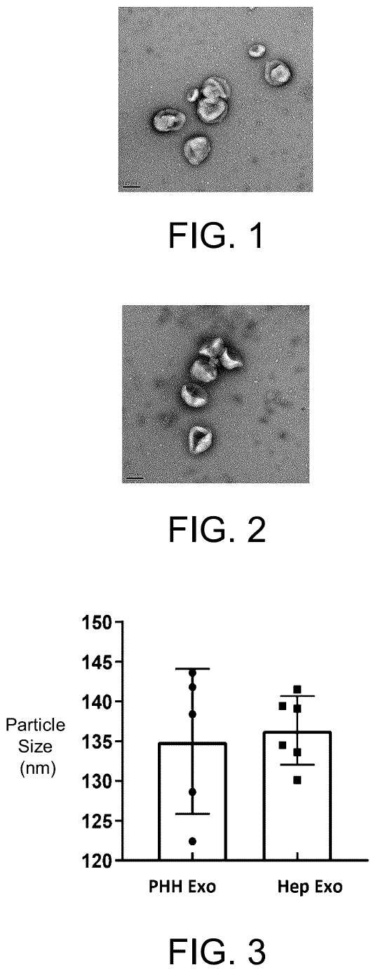

The PHH Exo sample and the Hep Exo sample were diluted and then fixed with a phosphate buffer containing 1% glutaraldehyde at a concentration of 0.1M, then added dropwise onto a copper grid, then negatively stained with 1% uranyl acetate, dried at room temperature, and then observed and photographed with a transmission electron microscope to obtain the transmission electron microscopic comparison photographs of the PHH Exo sample and the Hep Exo sample as shown in FIGS. 1 and 2 respectively, and the pH of the phosphate buffer was 7.4.

APMX110 nanoparticle tracking analyzer available from Particle Metrix, Germany was utilized to analyze the PHH Exo sample and the Hep Exo sample respectively, and the comparison chart of the average particle sizes of the particles in the two samples as shown in FIG. 3 was obtained. The specific detection and analysis methods were conventional technical means of those skilled in the art, and would not be repeated here.

Referring to FIGS. 1 and 2, the particles in the PHH Exo sample and the Hep Exo sample had a diameter of about 100 nanometers and presented with a round-like shape of regular morphology. Further referring to FIG. 3, the average particle size of the particles in the PHH Exo sample was 135±9.103 nm, and the average particle size of the particles in the Hep Exo sample was 136.4±4.323 nm, which conformed to the morphological characteristics of the exosomes.

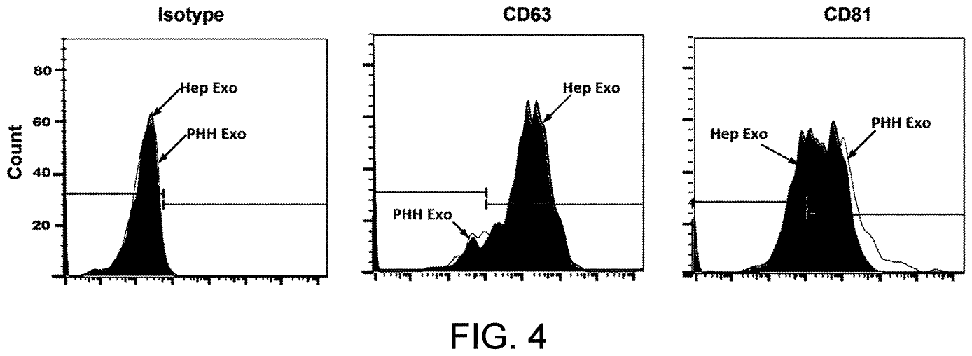

The PHH Exo sample and the Hep Exo sample were respectively diluted with a PBS solution and mixed evenly. A part of the mixture was stained with CD63-FITC and CD81-PE flow cytometry antibodies, and the other unstained part of the PHH Exo sample and the Hep Exo sample was used as a negative control. The aforementioned samples were tested on the Accuri C6 flow cytometer available from BD bioscience, USA to obtain the flow cytometry result shown in FIG. 4. The specific operation and analysis steps were conventional technical means of those skilled in the art, and would not be repeated here.

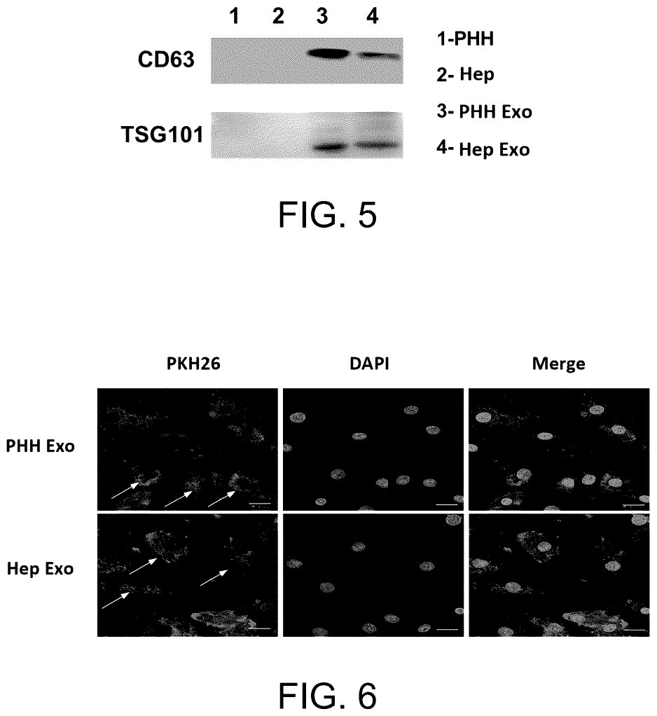

In this example, two cell products obtained after in vitro culture (abbreviated as PHH and Hep), the PHH Exo sample, the Hep Exo sample and a culture supernatant product were subjected to quantitative analysis of protein by a BCA method, and subjected to a Western blotting (WB) test to obtain the comparison photographs of the expression of CD63, CD81, TSG101, EEA1, GRP78 and β-actin in each sample as shown in FIG. 5. A method for lysing a cell product was: the culture supernatant was removed by pipetting, the cell pellet was washed with a PBS buffer and put into a 12-well plate, added with an appropriate amount of a RIPA lysis buffer to collect cells, and the cells were lysed on ice and then centrifuged at 4 degrees Celsius and 12,000 rpm for 10 min to collect the supernatant as a test sample. A SDS-PAGE protein loading buffer (5×) available from Beyotime Biotechnology Co., Ltd. and a Pierce™ BCA Protein Assay Kit available from Thermo Fisher, USA were used for protein quantification; and the WB test was carried out by using a highly sensitive ECL chemiluminescent detection kit available from Nanjing Vazyme Biotech Co., Ltd. and a ChemiDoc chemiluminescent imager available from BIO-RAD, USA. The specific operation and analysis steps were conventional technical means of those skilled in the art, and would not be repeated here.

Referring to FIG. 4, both the PHH Exo sample and the Hep Exo sample were positive in expression of exosome marker proteins CD63 and CD81, and according to statistics, the CD63 positive rates of the PHH Exo sample and the Hep Exo sample were 61.85±3.465% and 90.85±2.475%, respectively, and the CD81 positive rates of the PHH Exo sample and the Hep Exo sample were 69.90±4.95% and 89.40±1.273%, respectively. Referring to FIG. 5, the PHH Exo sample and the Hep Exo sample of a Exosome group both positive in expression of exosome marker proteins CD63 and TSG101, as compared with respective samples of the Cell Lysate group of cell lysates.

### Examples 1-2

In this example, the PHH Exo sample and the Hep Exo sample of Example 1-1 were labeled, and then co-cultured with PHHs to investigate the expression of exosomes in the cytoplasm of hepatocytes, proving that the exosomes derived from PHHs and HepLPCs can be successfully taken up by the hepatocytes.

With PHHs added with a PBS buffer solution as a negative control, the PHH Exo sample and the Hep Exo sample were diluted with a PBS buffer solution, the diluted PHH Exo sample and Hep Exo sample with different concentrations were respectively labeled with the PKH26 Red Fluorescent Cell Linker Kit available from Sigma, USA, and then co-incubated with the PHHs for 24 hours to complete the co-culture. After the co-culture was completed, the cell-containing culture was taken and fixed with a phosphate buffer solution containing 1% glutaraldehyde at a concentration of 0.1M, then stained with DAPI, and then observed under a fluorescent microscope to obtain the immunofluorescence confocal detection results of the PHH Exo sample and the Hep Exo sample after labeling, staining and co-culture as shown in FIG. 6. The specific labeling steps were provided by the kit. FIG. 6 showed that both the PHHs-derived exosomes and the HepLPCs-derived exosomes were significantly expressed in the cytoplasm of the hepatocytes. That was, they could be successfully taken up by the hepatocytes.

### Examples 1-3

This example provided use of a hepatocyte regulatory formulation containing exosomes in in vitro culture. Specifically, the PHH Exo sample and the Hep Exo sample of Example 1-1 were diluted with a buffer solution and co-cultured with the PHHs, and the obtained cells were analyzed for proliferation by BrdU ELISA detection and an EdU fluorescence method, and the expression of Ki67 positive cells was detected by immunofluorescence detection, proving that the PHHs-derived exosomes and the HepLPCs-derived exosomes could promote the proliferation of hepatocytes, and the HepLPCs-derived exosomes had more significant effect of promoting the proliferation of hepatocytes.

Firstly, the PHH Exo sample and the Hep Exo sample were respectively diluted with a PBS buffer to different concentrations of respectively 0, 1, 10, and 100 micrograms/milliliter, and respectively co-cultured with the PHHs for 24 hours at an PHHs inoculation density of 1×10⁵/square centimeter in a 12-well plate as the culture container.

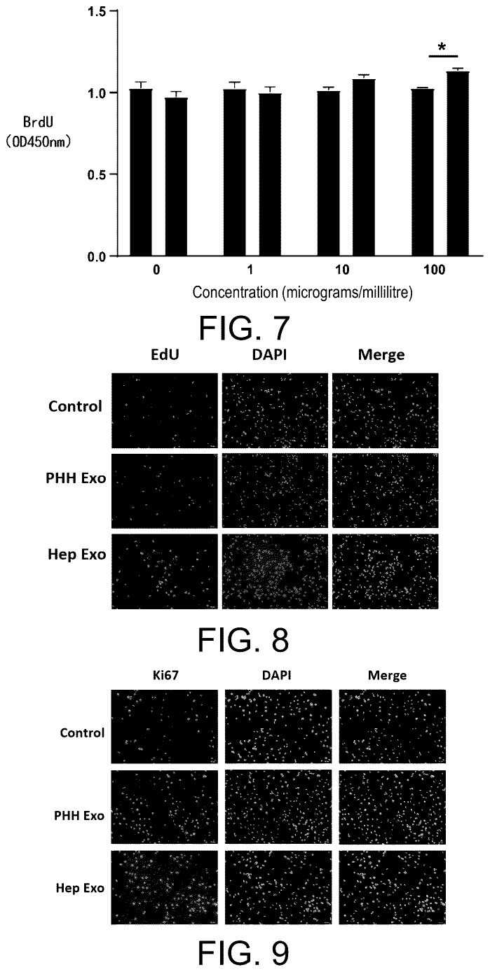

After the co-culture was completed, the BrdU Cell Proliferation ELISA Kit available from Abcam, Hong Kong was used for obtaining the OD value at a wavelength of 450 nanometers by ELISA detection, and the BrdU incorporation was further counted to obtain the comparison chart of BrdU incorporation of cells obtained after co-culture of the PHH Exo sample and the Hep Exo sample with different exosome concentrations as shown in FIG. 7, wherein: in the two histograms corresponding to each group of concentrations, the left side is the PHH Exo sample, and the right side is the Hep Exo sample. The specific detection steps were provided by the kit. ELISA detection and result statistical methods were conventional technical means of those skilled in the art.

After the co-culture was completed, the EdU Apollo 567 In Vitro Imaging Kit available from Guangzhou Ribo Biologics. Corporation was used for performing EdU labeling, cell fixation, Apollo staining and DNA staining on the cells obtained from co-culture of samples with an exosome concentration of 100 micrograms/milliliter. After DNA staining, the cells were observed by using a fluorescent microscope, and a PBS buffer was used as a negative control, to obtain the immunofluorescence photographs as shown in FIG. 8. The specific steps of EdU labeling, cell fixation, Apollo staining and DNA staining were provided by the kit.

After the co-culture was completed, Ki67 immunofluorescence staining was performed on each of the obtained cells to obtain the immunofluorescence photographs as shown in FIG. 9.

Referring to FIG. 7, the PHH Exo sample and Hep Exo sample with an exosome concentration of 100 micrograms/milliliter had a significant effect on the proliferation of the PHHs. Further referring to FIGS. 8 and 9, compared with the negative control group, both the PHH Exo sample and the Hep Exo sample promoted the proliferation of hepatocytes, and as obtained the statistics from FIGS. 8 and 9, for the Hep Exo sample and the PHH Exo sample, the EdU incorporation ratios were 19.89±1.049% and 27.09±30.308% respectively, and the percentages of Ki67 positive cells were 38.7±2.406% and 55.75±6.014% respectively.

EdU and BrdU were thymidine analogues, which infiltrated into DNA molecules being synthesized instead of thymine (T) during a DNA replication period, and were used for detecting DNA replication activity. Ki67 was an antigen associated with proliferating cells and was mainly used for labeling cells in a proliferation cycle. It could be seen from this example that since both the PHHs-derived exosomes and the HepLPCs-derived exosomes could promote the proliferation of hepatocytes, the hepatocyte regulatory formulation formed by combining any of the aforementioned exosomes with a diluent could be used as a medium for promoting the proliferation of hepatocytes.

In some embodiments, the diluent is a suspension with an exosome concentration of 10-200 micrograms/milliliter. In some specific embodiments, the resuspension is any one of a PBS buffer solution, physiological saline and a compound electrolyte solution.

### Examples 1-4

In this example, the cells obtained by co-culturing the HepLPCs-derived exosomes of Example 1-3 with the PHHs were taken as an example to analyze the cell cycle and the expression of cell cycle-related molecules, which proved that the HepLPCs-derived exosomes promoted the proliferation of hepatocytes by accelerating the progression of a cell cycle.

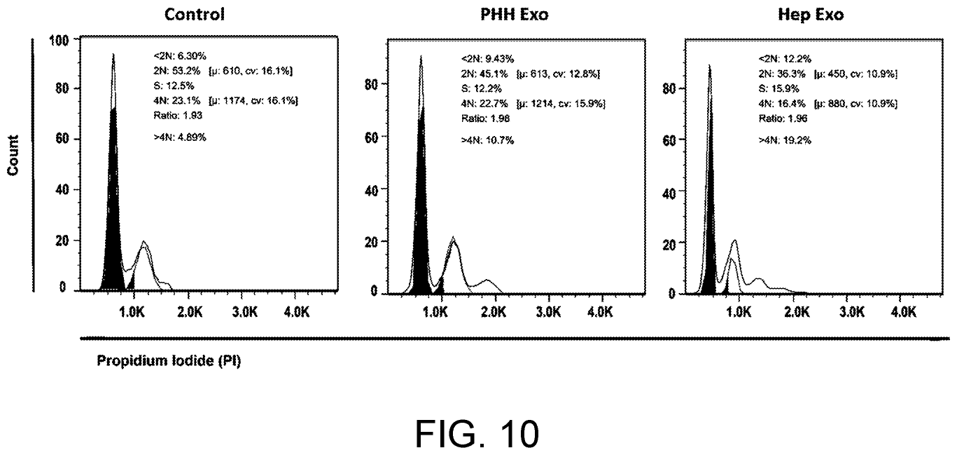

The cells obtained by co-culturing the PHH Exo sample and the Hep Exo sample of Example 1-3 with the PHHs (abbreviated as a PHH Exo-cell and a Hep Exo-cell respectively) were respectively digested with a pancreatin, analyzed for the cell cycle with a Propidium Iodide Flow Cytometry Kit available from Abcam, USA and a BD FACS Verse flow cytometer available from BD bioscience, USA, detected for the red fluorescence at an excitation wavelength of 488 nm, and meanwhile detected for light scattering. The cell DNA content and light scattering analysis were conducted by using Flowjo software to obtain the cell cycle analysis results as shown in FIG. 10, wherein the control group was the PHHs and a PBS buffer with an equal volume. The specific operation steps before loading onto a machine were provided by the kit.

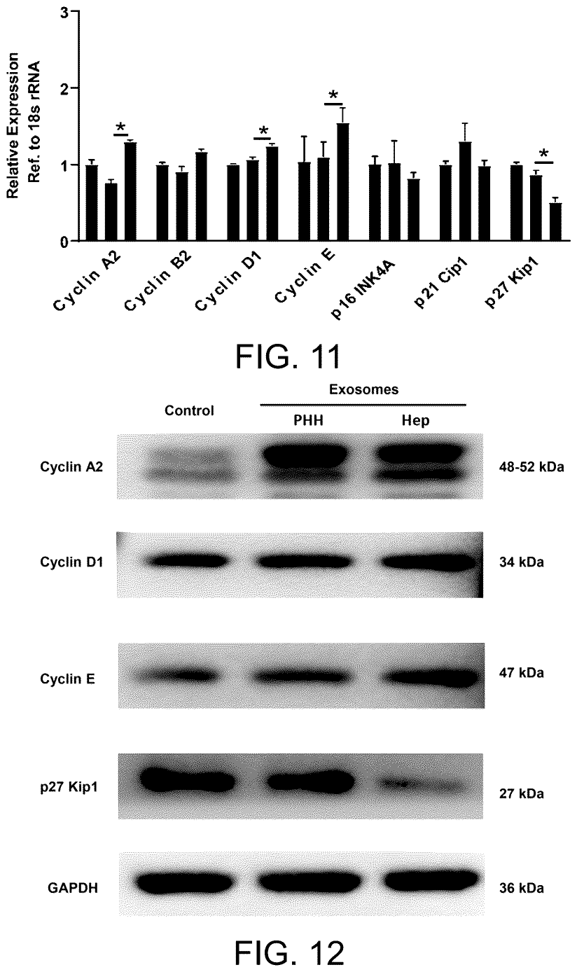

real-time fluorescent quantitative PCR analysis of the control group, the PHH Exo-cell and the Hep Exo-cell was conducted by using a HiScript III 1st Strand cDNA Synthesis Kit and a ChamQ SYBR Color qPCR Master Mix available from Nanjing Vazyme Biotech Co., Ltd. in a LightCycler480I real-time fluorescent quantitative PCR instrument available from Roche, Germany to obtained the comparison chart of miRNA expression levels of cell cycle-related molecules as shown in FIG. 11, wherein: the three histograms corresponding to each cell cycle-related molecule, were the control group, the PHH Exo sample and the Hep Exo sample from left to right sequentially.

A Western Blotting (WB) test was used for investigating the expression of the cell cycle-related molecules in the control group, the PHH Exo-cell and the Hep Exo-cell, so as to obtain the comparison chart of miRNA expression of the cell cycle-related molecules as shown in FIG. 12. For specific operation steps, please refer to the WB testing steps discussed in Example 1-1.

Referring to FIG. 10, compared with the control group and the PHH Exo-cell, for the Hep Exo-cell, the proportions of cells in a G0 phase and a G1 phase were decreased by 15.6±1.353% and 10.733±0.874% respectively (P<0.01), the proportions of cells in a S phase were increased by 6.47±0.97% and 4.17±1.527% respectively (P<0.01), the proportions of cells in a G2/M phase were increased by 14.9±1.413% and 9.133±2.101% respectively, indicating that the Hep Exo sample accelerated the transition process of primary hepatocytes from the G1 phase to the S phase and the G2/M phase, thereby promoting cell proliferation.

Referring to FIGS. 11 and 12, the expression of cell cycle-related proteins Cyclin A2, Cyclin D1, and Cyclin E was significantly up-regulated, while the expression of p27 kip1 was significantly down-regulated, indicating that the Hep Exo sample might promote the progression of the cell cycle by inhibiting the expression of p27 proteins while increasing cyclin family proteins, thereby promoting the proliferation of hepatocytes.

### Examples 1-5

In this example, the miRNAs of the exosomes in the PHH Exo sample and the Hep Exo sample of Example 1-1 were extracted, and it was proved by high-throughput sequencing analysis and sequencing bioinformatic analysis of exosome miRNAs, as well as a BrdU ELISA test and EdU proliferation analysis after in vitro transfection of the primary hepatocytes with miRNA mimics that, the miRNAs with significantly increased expression in the exosomes and capable of effectively promoting the proliferation of hepatocytes were hsa-miR-182, hsa-miR-183 and hsa-miR-574.

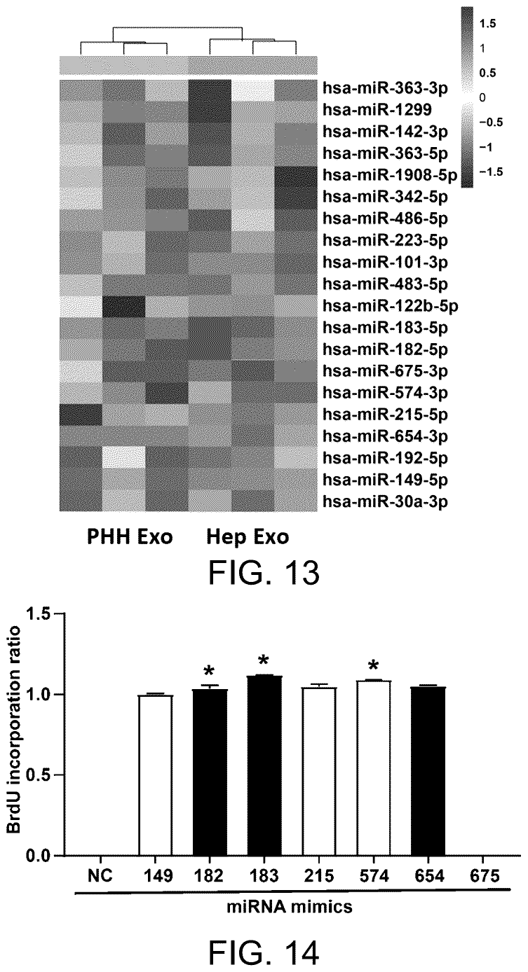

miRNAs were extracted by using a Total Exosome RNA and Protein Isolation Kit available from Invitrogen, USA to obtain a PHH Exo-derived analysis sample PHH-Exo-mi and a Hep Exo-derived analysis sample Hep Exo-mi, and then the miRNA 1st Strand cDNA Synthesis Kit, miRNA Universal SYBR qPCR Master Mix, HiScript III 1st Strand cDNA Synthesis Kit and ChamQ SYBR Color qPCR Master Mix available from Nanjing Vazyme Biotech Co., Ltd. were used for successively connecting linkers at the 3′ and 5′ terminals of the miRNAs, reverse transcribing into cDNAs, and then performing PCR amplification. After PCR amplification, a target fragment library was recovered by gel cutting, and the qualified library was sequenced and analyzed by the Illumina HiSeq™ 2500 high-throughput sequencer available from Illumina, USA, to obtain the clustering heat map as shown in FIG. 13, which provided up-regulated or down-regulated differential expression of the top 15 miRNAs.

In this example, 7 miRNAs with significantly up-regulated expression were selected and respectively denoted as hsa-miR-182, hsa-miR-183, hsa-miR-149, hsa-miR-215, hsa-miR-574, hsa-miR-654 and hsa-miR-675, the expression of these miRNAs was enhanced by in vitro transfection of the primary hepatocytes with miRNA mimics, and then they were detected for BrdU incorporation by BrdU ELISA, to obtain the comparison chart of BrdU incorporation in each cell transfected with each miRNA as shown in FIG. 14, wherein NC was a blank transfection group. The in vitro transfection was conducted with the miRNA mimic/inhibitor available from Guangzhou Ribo Biologics. Corporation. For the process of detecting BrdU incorporation by BrdU ELISA and the kit as used, please refer to Example 1-3.

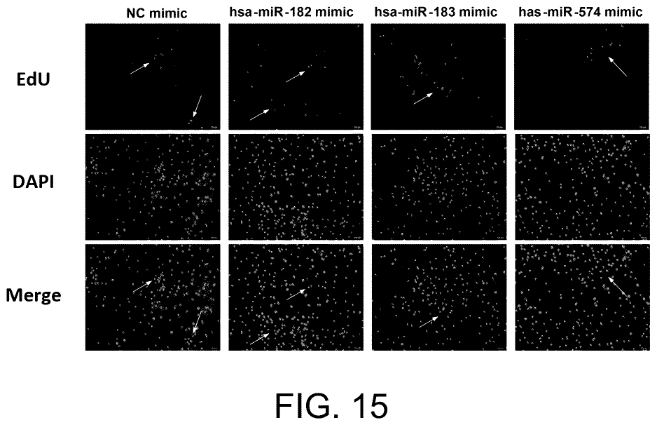

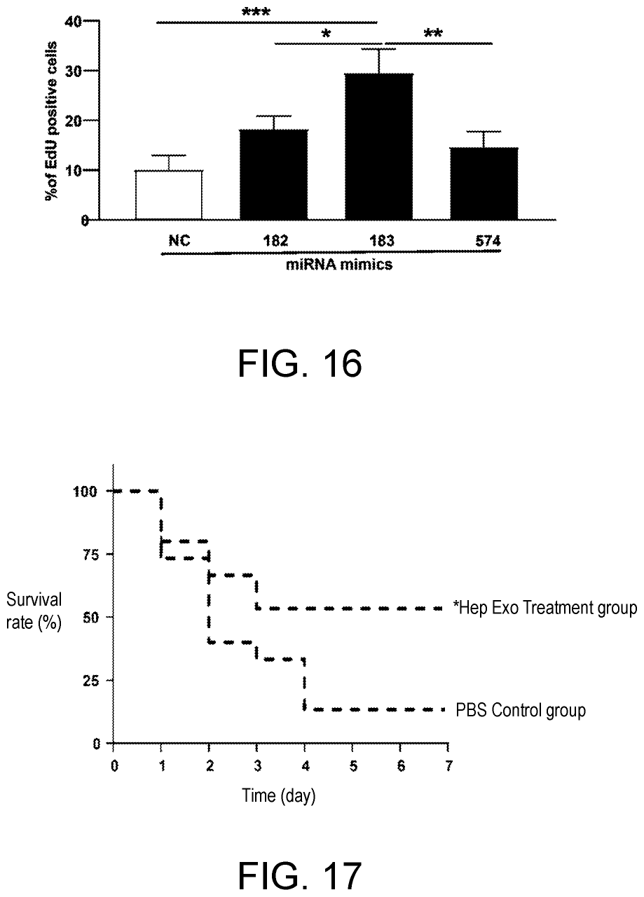

In this example, the primary hepatocytes transfected with hsa-miR-182, hsa-miR-183 and hsa-miR-574 were further stained with EdU, and then detected for the EdU incorporation rate by an EdU fluorescence method, so as to obtain the EdU fluorescence micrographs as shown in FIG. 15 and the comparison chart of EdU incorporation rate as shown in FIG. 16. For specific detection kits, please refer to Example 1-3.

Referring to FIG. 14, among the 7 miRNAs with significantly up-regulated expression, the hsa-miR-182, hsa-miR-183 and hsa-miR-574 had significant effect of promoting the proliferation of primary hepatocytes (p<0.05). Referring to FIGS. 15 and 16, compared with the group transfected with NC mimic, the EdU incorporation rate of the group transfected with hsa-miR-183 mimic in vitro was significantly increased, and was significantly higher than those of the group transfected with hsa-miR-182 and the group transfected with hsa-miR-574 mimic. The EdU incorporation rates of the group transfected with NC mimic, the group transfected with hsa-miR-182, the group transfected with hsa-miR-183 and the group transfected with hsa-miR-574 mimic were 10.04±2.946%, 18.22±2.67%, 29.46±4.799% and 14.6%±3.173%, respectively.

### Examples 1-6

In this example, after the establishment of a carbon tetrachloride-induced mouse acute liver failure model, the mice were subjected to tail vein injection of an injection containing the Hep Exo sample to serve as an in vivo animal model of liver failure intervened with an exosome formulation to promote regeneration of liver tissues.

Firstly, several C57BL/6 mice aged 6-8 weeks and weighing 22-25 g were used for establishing the carbon tetrachloride-induced mouse acute liver failure model and a control model. Particularly, the mice were injected intraperitoneally with an induction injection of carbon tetrachloride diluted with olive oil according to 1:4 to construct a mouse acute liver failure model, the mice were injected intraperitoneally with olive oil of an equal volume to construct a normal group, and the intraperitoneal injection dosage of the two models were each 1 mL/kg.

Then, 6 hours after the injection of the induction injection, partial of the mouse acute liver failure models were subjected to tail vein injection of a treatment injection obtained by mixing a PBS buffer with the Hep Exo sample evenly to form a group treated with Hep Exo; and partial of the mouse acute liver failure models were subjected to tail vein injection of a PBS buffer with the same volume as the treatment injection to form a PBS control group. In the treatment injection, the concentration of the Hep Exo sample was 2 micrograms/microliter. The injection doses of the group treated with Hep Exo and the PBS control group were both 15 mg/kg.

In some other embodiments, the injection doses of the group treated with Hep Exo and the PBS control group were 1-100 mg/kg.

All the aforementioned injections and buffers were sterilized and filtered through a 0.22 m filter before injection.

In this example, the survival condition of each mouse in the group treated with Hep Exo and the PBS control group were investigated within 7 days after the modeling was completed. Specifically, a Kaplan-meier method was used for survival analysis to plot a survival curve and perform a log-rank test with p<0.05 being considered to have a statistical difference, so as to obtain the comparison chart of survival condition of each mouse in the group treated with Hep Exo and the PBS control group as shown in FIG. 17, wherein the number of mice used for analysis in each group was 15. The specific analysis methods were conventional technical means of those skilled in the art, and would not be repeated here.

Referring to FIG. 17, the mice in the PBS control group had a 24-hour mortality rate of 25% and a mortality rate exceeding 50% within 48 hours, while the mice in the group treated with Hep Exo had a 24-hour mortality rate of 20% and a mortality rate of only 30% within 48 hours, and had no death after 72 hours (p<0.05), indicating that the exosomes derived from human liver precursor-like cells could improve the 7-day survival rate of the mice and had a significant therapeutic effect on acute liver failure.

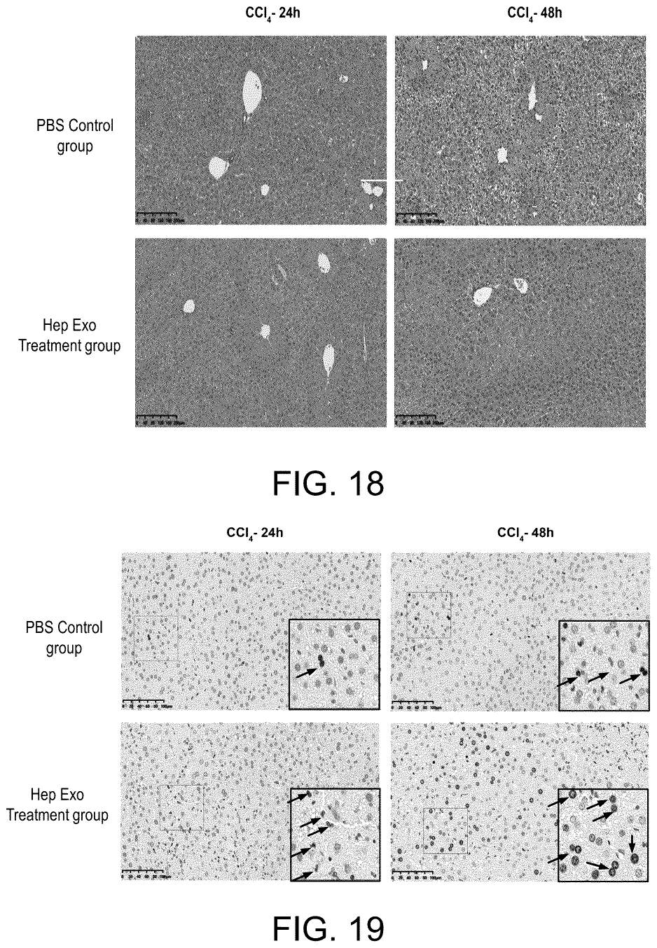

In this example, at 24 hours and 48 hours after modeling, the liver tissues of the mice in the normal control group, the PBS control group and the group treated with Hep Exo were taken, subjected to H&E (hematoxylin-eosin staining) staining and then prepared into sections. The sections were observed under a microscope, to obtain the comparison chart of the pathological damage of the mice in each group as shown in FIG. 18. The specific section preparation and observation steps were conventional technical means of those skilled in the art, and would not be repeated here.

Referring to FIG. 18, the liver tissues of the mice in the normal group showed normal hepatocellular morphology, had a complete and clear structure of the hepatic lobule, and had no inflammatory cell infiltration. Both at 24 h and 48 h of modeling, the mice in the PBS control group had obvious hepatocellular swelling, fragmented nuclei, increased vacuoles, local infiltration of inflammatory cells, damage to the normal connecting structure of hepatic cords, and severe hepatic sinusoidal congestion. However, both at 24 h and 48 h of modeling, the mice in the group treated with Hep Exo showed milder degree of hepatocellular damage, and had less vacuole formation and inflammatory cell infiltration than those of the control group and liver damage that was alleviated to a certain extent.

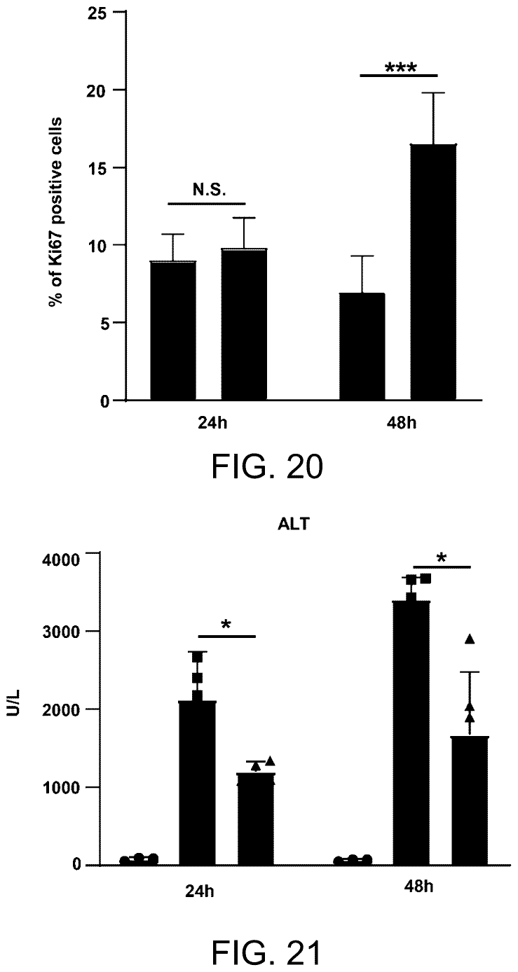

In this example, at 24 hours and 48 hours after modeling, the liver tissues of the mice in the normal group, the PBS control group and the group treated with Hep Exo were taken for paraffin sectioning, and then subjected to Ki67 immunohistochemical staining to investigate the regeneration of the liver tissues, so as to obtain the comparison chart of immunohistochemical Ki67 staining of the liver tissue sections of the mice in the PBS control group and the group treated with Hep Exo as shown in FIG. 19 and the percentage of Ki67 positive cells in the paraffin sections of the mouse liver tissues of each group as shown in FIG. 10, wherein the number of mice used for statistics in each group was 8. For specific steps of Ki67 immunohistochemical staining, please refer to Example 1-3. In FIG. 20, the two histograms corresponding to each modeling time were the PBS control group and the group treated with Hep Exo from left to right sequentially.

Referring to FIGS. 19 and 20, in the 48-hour sections of the mice in the group treated with Hep Exo, Ki67-positive expressing cells were significantly more than those in the PBS control group, and upon statistics, the percentages of Ki67-positive expressing cells in the group treated with Hep Exo and the PBS control group were respectively 16.587±3.381% and 7.021±2.415%, indicating that liver regeneration was effectively activated. It could be seen that Hep Exo sample had an important therapeutic effect in acute liver failure, could improve the survival rate of the mice, reduced liver damage, and effectively promoted liver tissue regeneration.

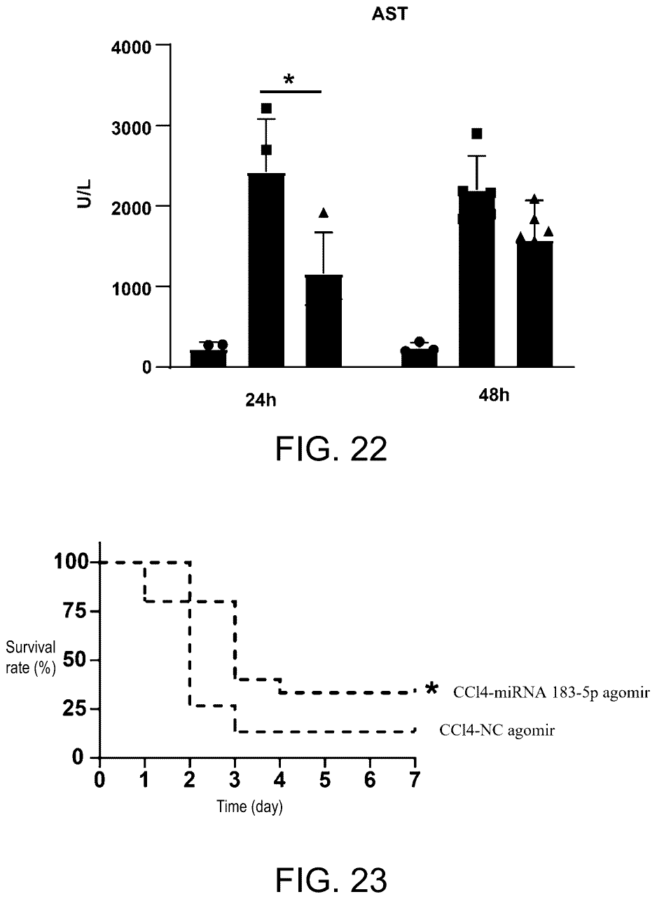

In this example, at 24 hours and 48 hours after modeling, the mice were anesthetized by inhalation of sevoflurane, the eyeballs of the mice were removed to take blood, and the blood was allowed to flow out naturally. The collected blood was collected into a 1.5 mL EP tube, allowed to stand at normal temperature for 30 min, and centrifuged at 4° C. and 3,000 rpm for 10 min, and then the supernatant was slowly pipetted as the mouse serum. An alanine aminotransferase (ALT) kit and an aspartate aminotransferase (AST) detection kit available from Beckman Coulter, USA were used for detecting biochemical indicators ALT and AST. The AST level and the ALT level were calculated according to the average absorbance ΔA per minute, so as to obtain the comparison results of the AST levels and the ALT levels of each mouse in the normal group, the PBS control group and the group treated with Hep Exo at different times as shown in FIGS. 21 and 22, wherein the number of mice for statistics in each group was 8. The three histograms corresponding to each modeling time in FIGS. 21 and 22 were the normal group, the PBS control group and the group treated with Hep Exo from left to right sequentially.

Referring to FIGS. 21 and 22, the levels of serum AST and ALT in the PBS control group and the group treated with Hep Exo were gradually increased after modeling and reached the peak at 48 h. Compared with the PBS control group, the levels of serum AST and ALT of mice in the group treated with Hep Exo were both significantly reduced. It could be seen that the HepLPC-derived exosomes could effectively reduce the levels of serum ALT and AST and play a protection role in acute liver injury in mice.

### Examples 1-7

In this example, miRNA-183 was taken as an example, and the use of an exosome formulation containing the miRNA-183 in the preparation of a drug for treating liver failure was provided.

Specifically, two mouse acute liver failure models induced by carbon tetrachloride and acetaminophen were constructed to simulate different liver injury mechanisms respectively. The mice were subjected to tail vein injection of an injection containing the miRNA-183 to serve as an in vivo animal model of liver failure intervened with an exosome formulation to promote regeneration of liver tissues.

A method for constructing the carbon tetrachloride-induced acute liver failure model (abbreviated as a CCl4 model) was: C57BL/6 mice aged 6-8 weeks and weighing 22-25 g were intraperitoneally injected with a carbon tetrachloride induction injection obtained by diluting carbon tetrachloride with olive oil according to 1:4 at an injection dose of 1 mL/kg. The corresponding normal control group was intraperitoneally injected with an equal volume of olive oil (abbreviated as a CCl4 normal group). The carbon tetrachloride induction injection was sterilized and filtered with a 0.22 micron filter before injection.

A method for constructing the acetaminophen-induced acute liver failure model (abbreviated as an APAP model) was: C57BL/6 mice aged 6-8 weeks and weighing 22-25 g were intraperitoneally injected with an acetaminophen induction injection obtained by mixing acetaminophen with a PBS buffer at an injection dose of 1 mL/kg. The corresponding normal control group was intraperitoneally injected with an equal volume of the PBS buffer (abbreviated as an APAP normal group). The acetaminophen induction injection was sterilized and filtered with a 0.22 micron filter before injection.

After 6 hours of modeling, partial of the CCl4 model and partial of the APAP model were subjected to tail vein injection of each 200 microliters of a negative control injection diluted with CCl4 and the PBS buffer respectively and containing 15 nanomoles of miRNA-183 agomir (abbreviated as a CCl4-NC agomir group and an APAP-NC agomir group respectively).

Cy5-labeled miRNA-183 agomir was encapsulated with an Entranster™-in vivo RNA transfection reagent and diluted with CCl4 and a PBS buffer respectively, and then after 6 hours of modeling, partial of the CCl4 model and partial of the APAP model were subjected to tail vein injection (abbreviated as a CCl4-miRNA 183-5p agomir group and an APAP-miRNA 183-5p agomir group respectively). The injection contained 2 micrograms of nucleic acid per microliter of the transfection reagent, the injection volume of each injection was 200 microliters, and the content of the miRNA-183 agomir was 15 nanomoles. The Entranster™-in vivo RNA transfection reagent was available from Engreen Biosystem Co, Ltd., Beijing, and the Cy5-labeled miRNA-183 agomir was available from Guangzhou Ribo Biologics. Corporation.

The liver tissues and blood samples were taken from each group at different time points after modeling to detect indicators related to liver injury and regeneration. The number of mice used for detection in each group was 8. The details were as follows:

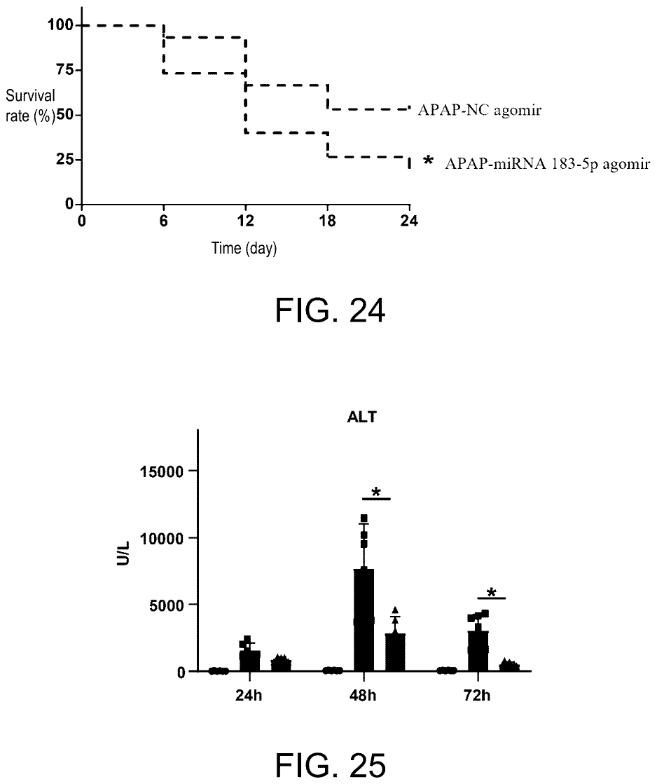

This example investigated the 7-day survival rate of mice subjected to different treatment after modeling, so as to obtain the 7-day survival rate of mice in each group of the CCl4 model and the 7-day survival rate of mice in each group of the APAP model as shown in FIGS. 23 and 24 respectively. For specific operation and analysis processes, please refer to Example 1-3.

Referring to FIG. 23, the mice in the CCl4-NC agomir group had a 24-hour mortality rate of 20%, and a mortality rate exceeding 70% within 48 hours, while the mice in the CCl4-miRNA 183-5p agomir group did not die within 24 hours and had a mortality rate of only 20% within 48 hours (p<0.05); and referring to FIG. 24, the mice in the APAP-NC agomir group had a 24-hour mortality rate up to 80%, while the mice in the APAP-miRNA 183-5p agomir group had a 24-hour mortality rate of only 50%. FIG. 23 and FIG. 24 both showed that miRNA 183-5p could significantly improve the 7-day survival rate of the mice, and had a significant therapeutic effect on acute liver failure.

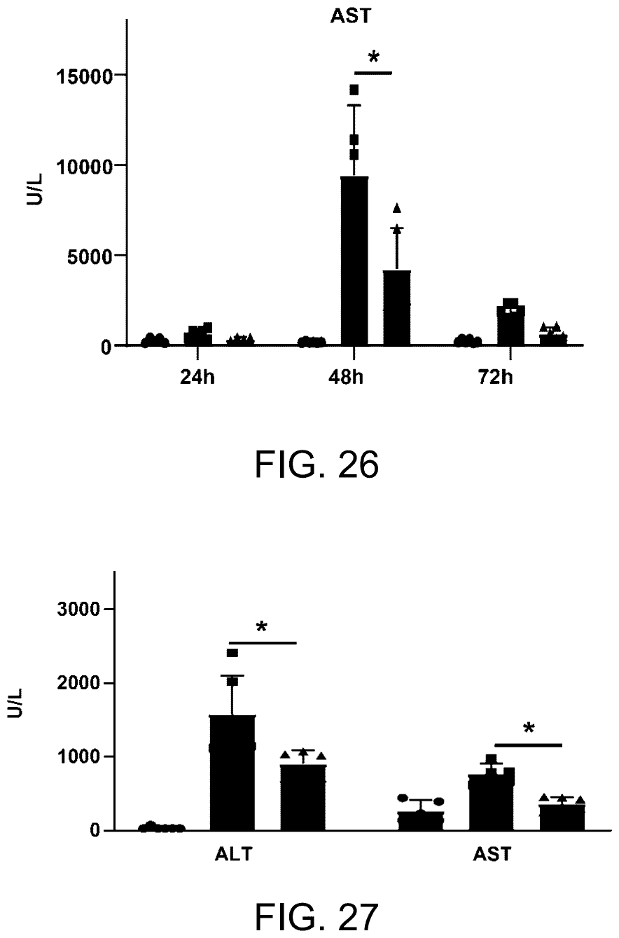

In this example, the peripheral blood of each mouse of the CCl4 model was taken at different time points within 72 hours after modeling for detection of ALT and AST, and the peripheral blood of each mouse of the APAP model was taken for detection of ALT and AST within 24 hours after modeling, so as to obtain the comparison charts of the AST level and the ALT level respectively as shown in FIGS. 25 to 27. For the specific detection processes of ALT and AST, please refer to Example 1-3. In FIGS. 25 and 26, the three histograms corresponding to each modeling time were the normal group, the CCl4-NC agomir group and the CCl4-miRNA 183-5p agomir group from left to right sequentially. In FIG. 27, the three histograms corresponding to each factor (ALT or AST) were the normal group, the APAP-NC agomir group and the APAP-miRNA 183-5p agomir group from left to right sequentially.

Referring to FIGS. 25 and 26, the levels of AST and ALT of the mice in the CCl4-NC agomir group and the mice in the CCl4-miRNA 183-5p agomir group were both gradually increased after modeling and reached the peak at 48 h, but were reduced at 72 h; while the levels of AST and ALT of the mice in the CCl4-miRNA 183-5p agomir group were significantly reduced compared with those of the mice in the CCl4-NC agomir group. Referring to FIG. 27, the AST and ALT levels of the mice in the APAP-miRNA 183-5p agomir group were significantly lower than those of the mice in the APAP-NC agomir group. All of the above showed that miRNA 183-5p could effectively reduce the levels of serum ALT and AST.

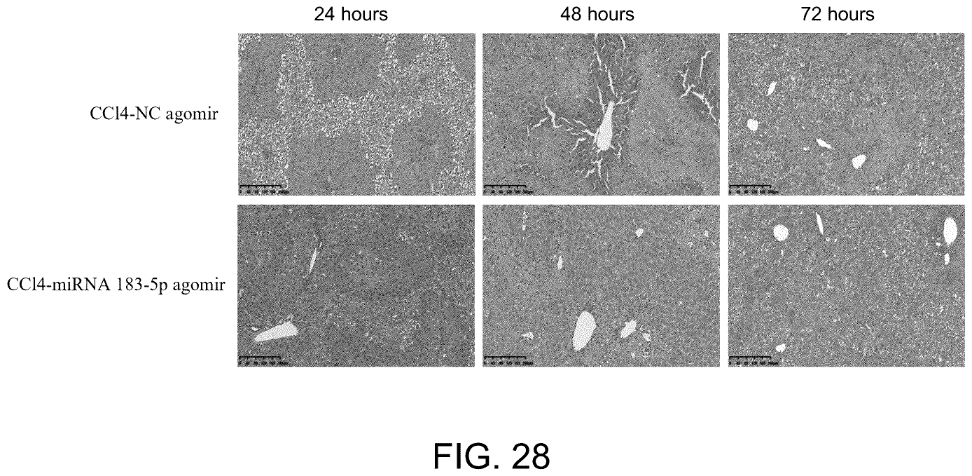

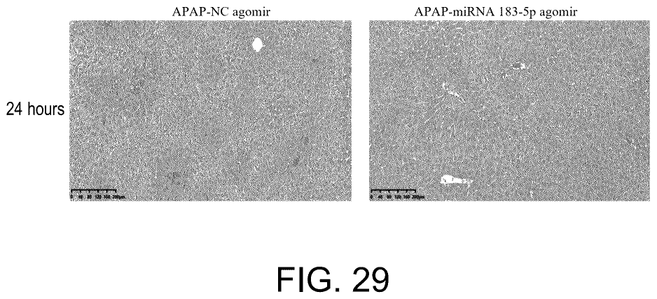

In this example, the liver tissues of each mouse of the CCl4 model were taken at different time points after modeling for pathological HE staining to investigate liver injury, and the liver tissues of each mouse of the APAP model were taken at 24 hours after modeling for pathological HE staining to investigate liver injury, so as to obtain the comparison chart of the pathological tissue sections of each mouse of the CCl4 model and each mouse of the APAP model after modeling as shown in FIGS. 28 to 29 respectively. For the specific operation processes, please refer to Example 1-3.

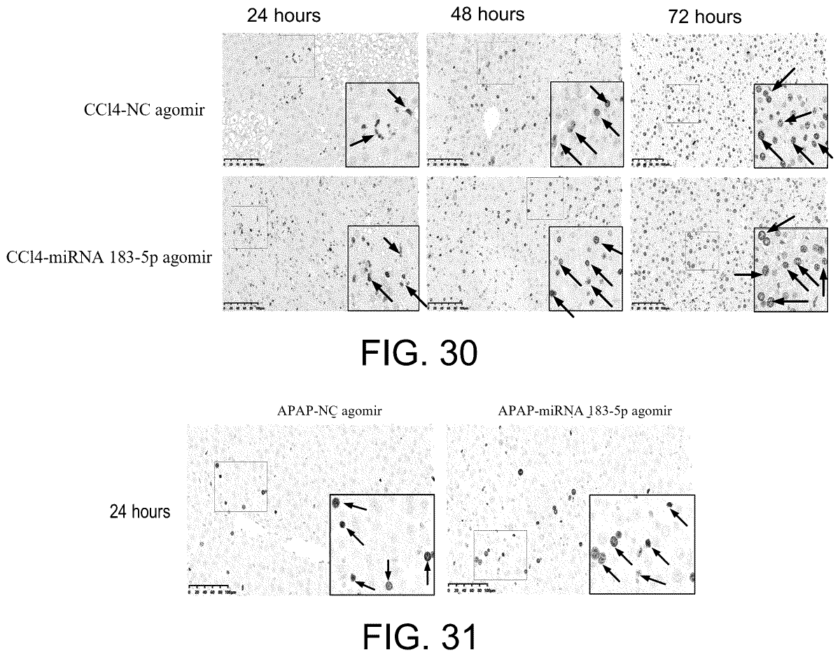

In this example, the expression of Ki67 was further detected immunohistochemically to evaluate the liver tissue regeneration of differently treated mice, so as to obtain the comparison chart of immunohistochemical Ki67 staining of the liver tissue sections of the mice in the CCl4-NC agomir group and the mice in the CCl4-miRNA 183-5p agomir group at different time points after modeling as shown in FIG. 30, and the comparison chart of immunohistochemical Ki67 staining of the liver tissue sections of the mice in the APAP-NC agomir group and the mice in the APAP-miRNA 183-5p agomir group 24 hours after modeling as shown in FIG. 31. For the specific operation processes, please refer to Example 1-3.

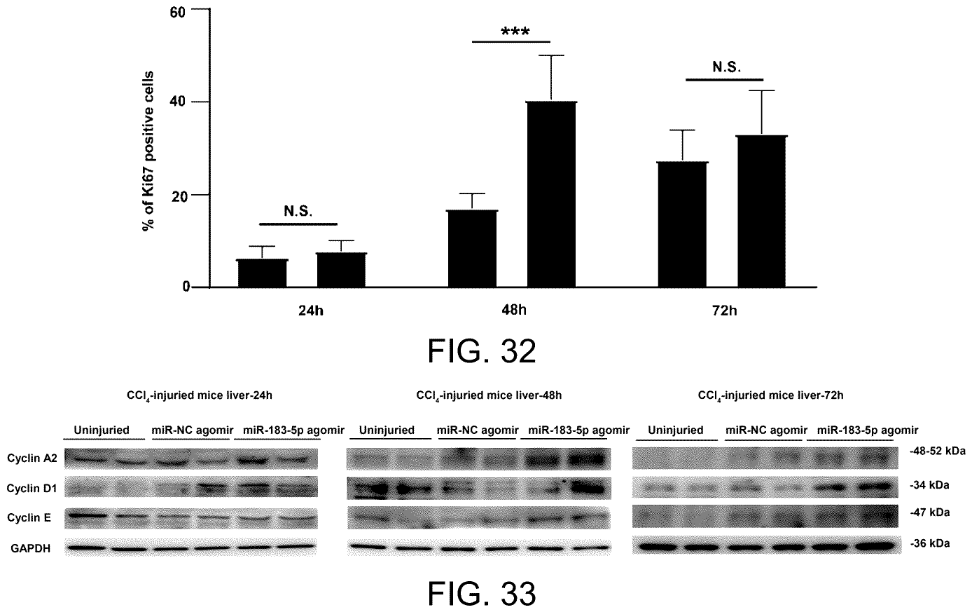

FIG. 32 counted the percentages of Ki67 positive cells in the liver tissue sections of the mice in the CCl4-NC agomir group and the mice in the CCl4-miRNA 183-5p agomir group at different times after modeling. Among them: the two histograms corresponding to each modeling completion time were the CCl4-NC agomir group and the CCl4-miRNA 183-5p agomir group from left to right sequentially.

Referring to FIGS. 28 to 32, the liver tissues of the mice in the CCl4 normal group and the APAP normal group both showed normal hepatocellular morphology, had a complete and clear structure of the hepatic lobule, and had no inflammatory cell infiltration. The mice in the CCl4-NC agomir group and the APAP-NC agomir group showed obvious liver injury occurred at 24 hours after modeling, and showed severe liver cell necrosis, fragmented nuclei, increased vacuoles, a large number of inflammatory cell infiltration, and severe hepatic sinusoidal congestion occurred at 48 h. However, the mice in the CCl4-miRNA 183-5p agomir group and the APAP-miRNA 183-5p agomir group also showed liver injury 24 hours after modeling, but after 48 hours of modeling, the degree of liver injury was significantly alleviated, and vacuole formation and inflammatory cell infiltration were both less than those of the corresponding NC agomir group.

Compared with the CCl4-NC agomir group, the number of Ki67-positive expressing cells in the 48-hour sections of the mice in the CCl4-miRNA 183-5p agomir group was significantly increased (p<0.05); and compared with the APAP-NC agomir group, the number of Ki67-positive expressing cells in the 24-hour sections of the mice in the APAP-miRNA 183-5p agomir group was significantly increased (p<0.05), indicating that the liver regeneration was effectively activated.

In this example, the protein expression of cell cycle-related molecules of each mouse in the CCl4-miRNA 183-5p agomir group at different times after the completion of modeling was investigated by a Western blotting method, so as to obtain the schematic diagram as shown in FIG. 33. For the specific operation methods, please refer to Example 1.

Referring to FIG. 33, the expression of cell cycle-related proteins Cyclin A2, Cyclin D, and Cyclin E in the liver tissues of the CCl4-miRNA 183-5p agomir group was up-regulated, while the expression of p27 kip1 was down-regulated, indicating that miRNA-183-5p might promote the proliferation of hepatocytes by inhibiting the expression of the p27 protein while increasing the cyclin family protein to promote the progression of the cell cycle.

In the following examples, an anti-hepatic fibrosis formulation was prepared as a liver disease regulatory formulation, and the use of such a liver disease regulatory formulation was investigated. The details were as follows:

### Examples 2-1

This example provided the first anti-hepatic fibrosis formulation, the preparation method of which was as follows:
S0: human liver precursor-like cells Human-HepLPCs with a confluence of no less than 60% were provided as seed cells;
S1: the Human-HepLPCs were cultured in vitro with a serum-free DMEM medium for 24 hours; and
S2: the in vitro culture supernatant was collected, and subjected to 25-fold filtration and concentration after cell debris in the in vitro culture supernatant was removed, so as to obtain the secretory supernatant as an anti-hepatic fibrosis formulation. Specifically, in the step S2, the cell debris in the culture supernatant was removed under a centrifugal force of 3,000 g; and filtration and concentration was conducted with a 10 kDa Amicon Ultra ultrafilter.

In this example, the total protein content in the aforementioned secretory supernatant was 2.2 milligrams/milliliter, as detected by the detection method provided in the instructions employing a BCA protein quantification kit (available from Shanghai beyotime Biotechnology Co., Ltd.).

The Human-HepLPCs in the step S0 of this example were obtained by conducting transformation and expansion culture of Human-primary hepatocytes in a TEM medium for 7-9 days, and then subjecting to 1:(3-6) subculture to the 2nd-5th generation. Specifically, the TEM medium was composed of the following components: a DMEM/F12 basal medium, as well as based on the content in the DMEM/F12 basal medium: a N2 nutrient supplement (100×) with a content of 1%, a B27 nutritional supplement (50×) with a content of 1%, 1 mM sodium pyruvate, 10 μg/mL of ascorbic acid, 20 ng/mL of a hepatocyte growth factor HGF, 20 ng/mL of an epithelial growth factor EGF, 10 μM of a ROCK kinase inhibitor Y27632, 3 μM of a Wnt signaling pathway agonist CHIR99021, 1 μM of a TGF-β signaling inhibitor A8301, 1 μM of sphingosine-1-phosphate S1P and 5 μM of indoleacetic acid LPA. Among them, the DMEM/F12, the N2 nutritional supplement, the B27 nutritional supplement and the sodium pyruvate were available from Invitrogen; the ascorbic acid was available from Sigma-Aldrich; the HGF and the EGF were available from Novoprotein; the Y27632, CHIR99021, A8301, s1p and LPA were all available from TargetMol.

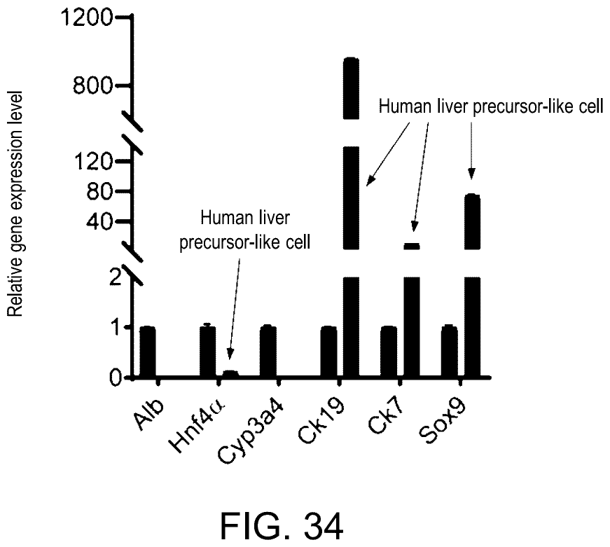

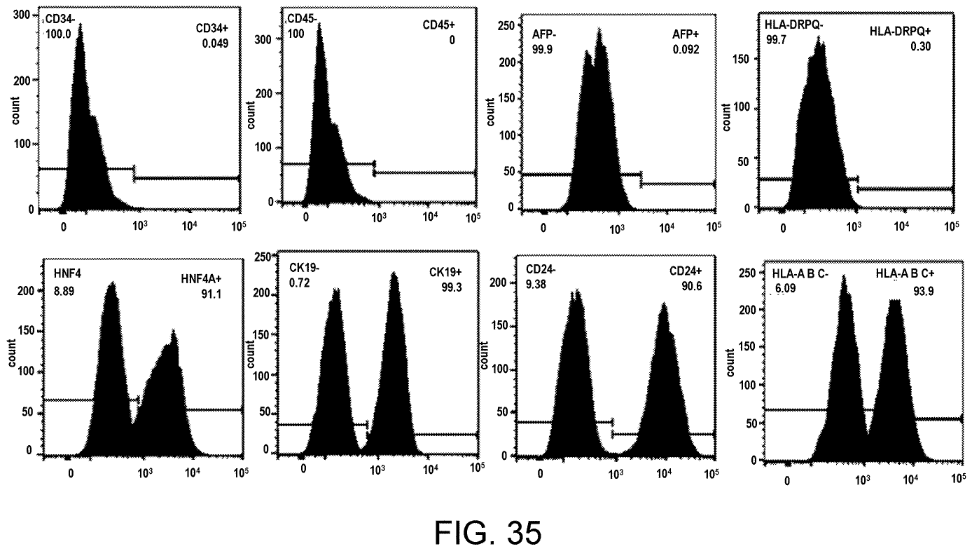

In this example, qPCR and flow cytometry were used for analyzing the gene expression of human primary hepatocytes and human liver precursor-like cells, and the results were shown in FIGS. 34 and 35. Referring to FIG. 34, the histogram indicated by the arrow represented the relative gene expression of the human liver precursor-like cells. Under the action of the TEM medium, in the human primary hepatocytes, the expression of liver progenitor cell genes Ck7, Ck19 and Sox9 were increased significantly, while the expression of liver parenchymal cell markers such as Alb, Cyp3a4 and Hnf4α were decreased significantly. Referring to FIG. 35, in the human liver precursor-like cells, the liver cell marker HNF4α and liver stem cell/hepatic progenitor cell markers CD24 and CK19 were significantly expressed, and the expression levels of a hematopoietic stem cell antigen CD34, a leukocyte common antigen CD45 and a liver fetal cell marker AFP were all less than 2%. The human liver precursor-like cells did not express the MHC class II antigens HLA-DP, HLA-DQ and HLA-DR, showing low immunogenicity.

### Example 2-2

In this example, the first anti-hepatic fibrosis formulation of Example 2-1 was co-cultured with the human immortalized hepatic stellate cell line LX-2, to investigate the apoptosis-promoting effect of the anti-hepatic fibrosis formulation on LX-2. LX-2 in this example was purchased from Procell.

The process of co-culturing the first anti-hepatic fibrosis formulation of Example 1 with LX-2 was as follows: LX-2 was fixed in a DMEM medium containing 10% FBS, 100 U/mL of penicillin and 100 mg/mL of streptomycin, added with 2.5 ng/mL of TGF-β1 to activate LX-2; and then added with the anti-hepatic fibrosis formulation of Example 1, mixed evenly, and allowed to stand for 48 hours. In the co-culture mixture, the volume percentage content of the first anti-hepatic fibrosis formulation was 1%, 2.5% and 5%. Among them, the first anti-hepatic fibrosis formulation was added with 10 μg/mL of an anti-FGF19 antibody (rabbit monoclonal antibody, available from R&D Systems) and 10 μg/mL of an anti-AREG antibody (rabbit polyclonal antibody, available from R&D Systems) and incubated for 2 hours first before use.

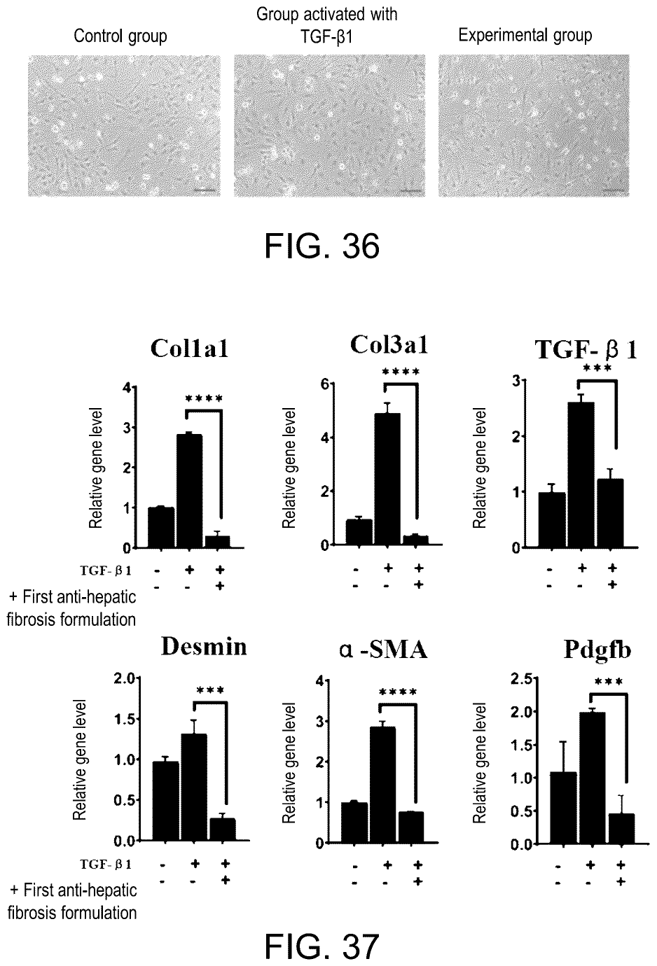

In this example, the aforementioned cells obtained by co-culture were used as the experimental group, the cells obtained by co-culturing LX-2 with the DMEM medium for 48 hours were used as the control group, and the cell aggregate obtained after LX-2 was activated with the addition of TGF-β1 and then co-cultured with the DMEM medium for 48 hours was used as the group activated with TGF-β1. The morphology of the three groups was observed with a transmission electron microscope after sample preparation to obtain the photographs of microscopic morphology of the LX-2 cells in each group as shown in FIG. 36. Referring to FIG. 3, activation of the LX-2 cells by TGF-β1 resulted in a change in the morphology of the LX-2 cells into elongated dendrites, and the addition of the first anti-hepatic fibrosis formulation for co-culture could reverse this change.

In this example, the relative mRNA expression of HSCs activation-related genes Col1a1, Col3a1, TGF-β1, Desmin, α-SMA and Pdgfb in LX-2 of the group activated with TGF-β1 and the experimental group (the volume percentage of the first anti-hepatic fibrosis formulation being 1%) was investigated by a real-time polymerase chain reaction (RT-PCR), the data was normalized to GAPDH expression and compared with the control group to count the degree of differentiation, so as to obtain the comparison chart of relative mRNA expression of HSCs activation-related genes in each group as shown in FIG. 37. It could be seen from FIG. 37 that TGF-β1 activated LX-2 cells, making the expression levels of the aforementioned HSCs activation-related genes be up-regulated; while the introduction of the first anti-hepatic fibrosis formulation significantly inhibited the expression levels of the aforementioned HSCs activation-related genes. It could be seen that the first anti-hepatic fibrosis formulation had a significant inhibitory effect on the activation of HSCs.

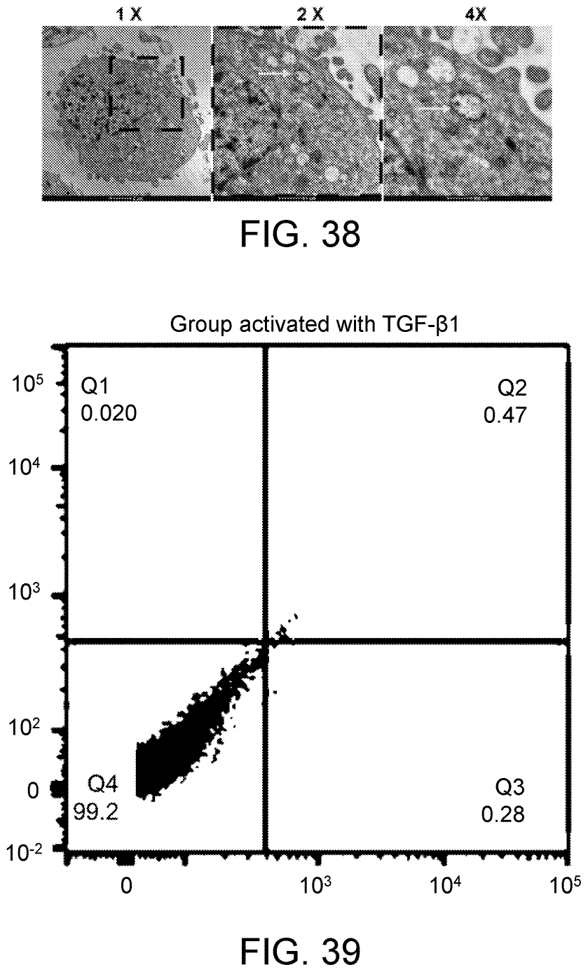

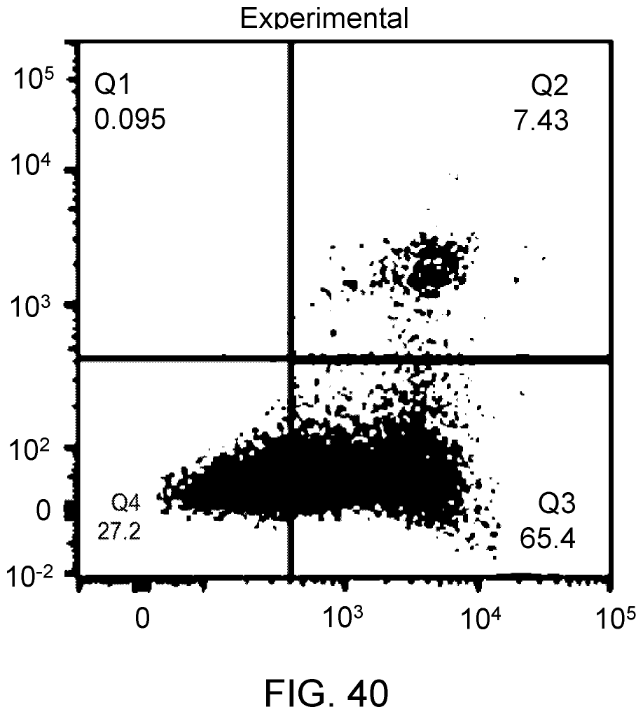

In this example, the cells of the experimental group (the volume percentage content of the first anti-hepatic fibrosis formulation being 1%) were made into a sample and then observed under a transmission electron microscope, to obtain the microscopic photograph as shown in FIG. 38. Referring to FIG. 38, apoptotic bodies indicated by the arrows were observed in the cell aggregates obtained by co-culturing the first anti-hepatic fibrosis formulation with LX-2. The cells obtained from the experimental group and the group activated with TGF-β1 were further stained with annexin V/PI and then detected by flow cytometry to obtain the flow cytometry diagram of the control group as shown in FIG. 39 and flow cytometry diagram of the experimental group as shown in FIG. 40. Referring to FIGS. 39 and 40, the first anti-hepatic fibrosis formulation induced HSC apoptosis.

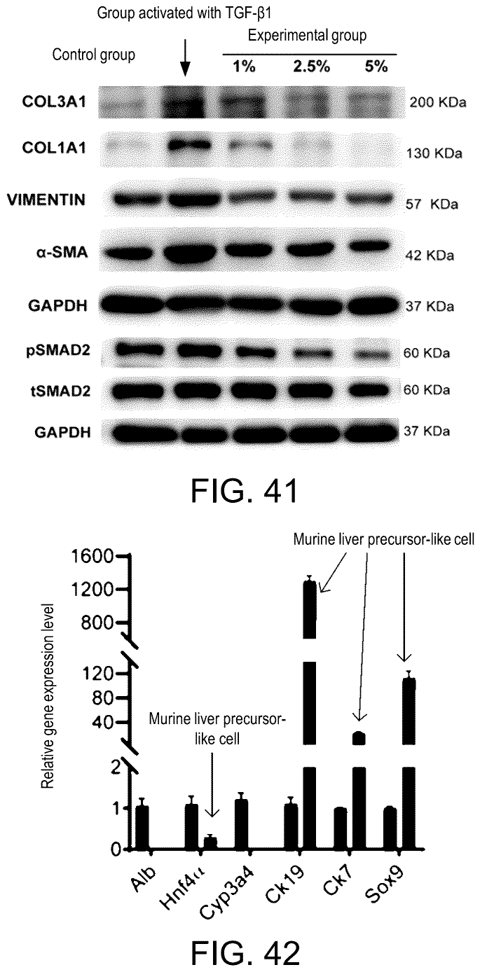

In this example, the cell aggregates of the control group, the group activated with TGF-β1 and the experimental groups with different mass percentages of anti-hepatic fibrosis formulations were subjected to Western blot analysis, so as to obtain a comparison chart of the expression of fibrosis-related proteins and the expression of key fibrosis signals of cells in each group as shown in FIG. 41. It could be seen that in the presence of TGF-β1, the first anti-hepatic fibrosis formulation induced a dose-dependent decrease in the expression of the fibrosis-associated proteins and the key fibrosis signal TGF-β-SMAD pathway.

### Examples 2-3

This example provided a second anti-hepatic fibrosis formulation, and its preparation method was to take murine liver precursor-like cells Rat-HepLPCs with confluence of no less than 60% as seed cells, and for the other preparation processes, please refer to Example 1.

The Rat-HepLPCs were obtained by culturing the murine primary hepatocytes (Rat primary hepatocytes) as seed cells in a TEM medium. For the specific culture process, please refer to Example 2-1.

In this example, the gene expression of the Rat primary hepatocytes and the Rat-HepLPCs were analyzed by qPCR and flow cytometry, and the results were as shown in FIG. 42. Referring to FIG. 42, under the action of the TEM medium, in the murine liver precursor-like cells, the expression of liver progenitor cell genes Ck7, Ck19 and Sox9 were increased significantly, while the expression of liver parenchymal cell markers such as Alb, Cyp3a4 and Hnf4α were decreased significantly.

### Example 2-4

In this example, the second anti-hepatic fibrosis formulation of Example 2-3 was co-cultured with the murine hepatic stellate cell line HSCs-T6, to investigate the apoptosis-promoting effect of the anti-hepatic fibrosis formulation on HSCs-T6. The HSCs-T6 in this example was purchased from Procell.

For the process of co-culturing the second anti-hepatic fibrosis formulation of Example 2-3 with HSCs-T6, please refer to Example 2-2. In the co-culture mixture, the volume percentage content of the second anti-hepatic fibrosis formulation was 1%.

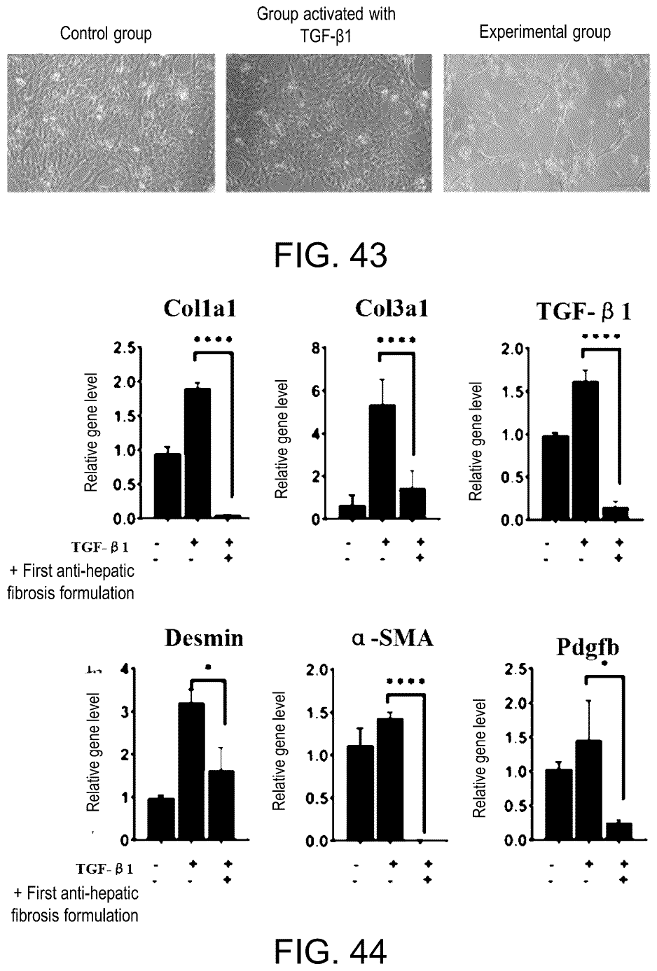

In this example, the cells obtained by co-culturing the second anti-hepatic fibrosis formulation with HSCs-T6 were used as the experimental group, the cells obtained by co-culturing HSCs-T6 with the DMEM medium for 48 hours were used as the control group, and the cell obtained after HSCs-T6 was activated with the addition of TGF-β1 and then co-cultured with the DMEM medium for 48 hours was used as the group activated with TGF-β1. The morphology of the three groups was observed with a transmission electron microscope after sample preparation to obtain the photographs of microscopic morphology of the HSCs-T6 cells in each group as shown in FIG. 43. Referring to FIG. 43, activation of the HSCs-T6 cells by TGF-β1 resulted in a change in the morphology of the HSCs-T6 cells into elongated dendrites, and the addition of the first anti-hepatic fibrosis formulation for co-culture could reverse this change.

In this example, the relative mRNA expression of HSCs activation-related genes Col1a1, Col3a1, TGF-β1, Desmin, α-SMA and Pdgfb in HSCs-T6 of the control group, the group activated with TGF-β1 and the experimental group was investigated by a real-time polymerase chain reaction (RT-PCR), the data was normalized to GAPDH expression and compared with the control group to count the degree of differentiation, so as to obtain the comparison chart of relative mRNA expression of HSCs activation-related genes in each group as shown in FIG. 44. It could be seen from FIG. 44 that TGF-β1 activated HSCs-T6 cells, making the expression levels of the aforementioned HSCs activation-related genes be up-regulated; while the introduction of the second anti-hepatic fibrosis formulation significantly inhibited the expression levels of the aforementioned HSCs activation-related genes. It could be seen that the second anti-hepatic fibrosis formulation had a significant inhibitory effect on the activation of HSCs.

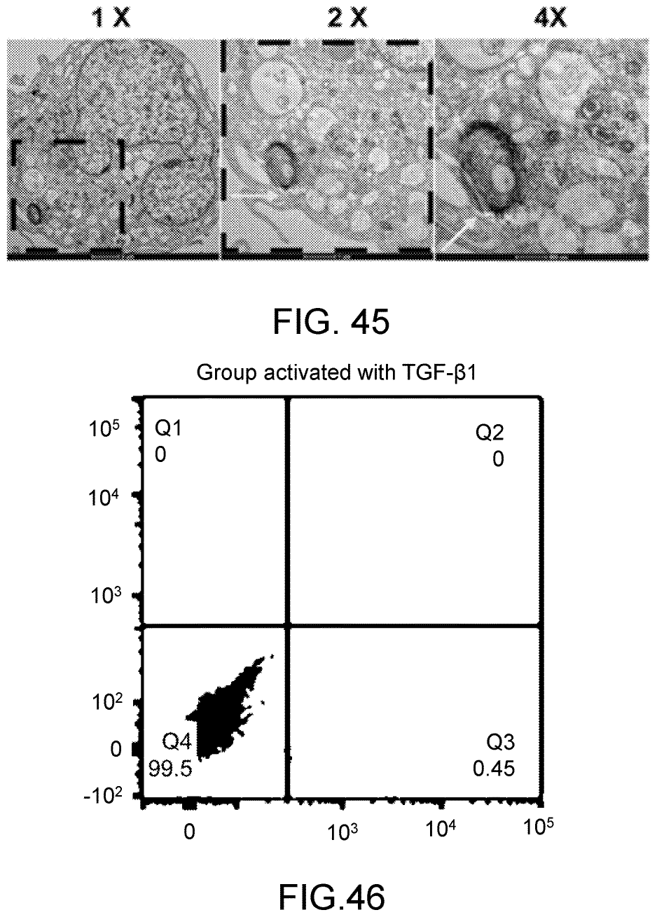

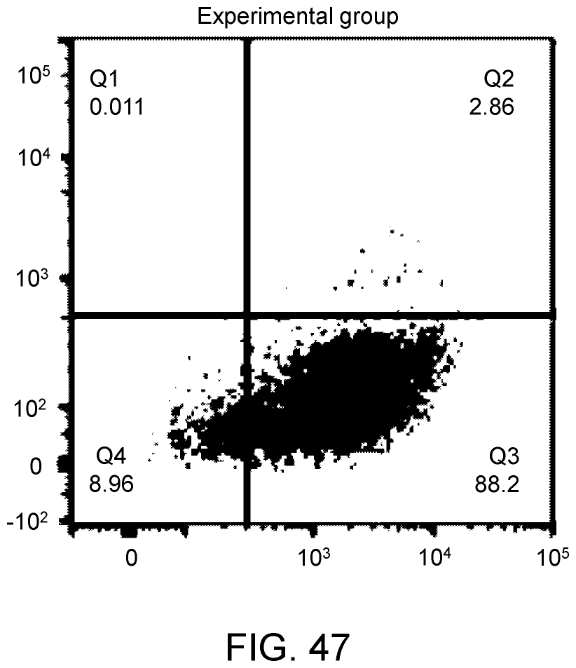

In this example, the cells of the experimental group were made into a sample and then observed under a transmission electron microscope, to obtain the microscopic photograph as shown in FIG. 45. Referring to FIG. 45, apoptotic bodies indicated by the arrows were also observed in the cells obtained by co-culturing the second anti-hepatic fibrosis formulation with HSCs-T6. The cells obtained from the experimental group and the group activated with TGF-β1 were further stained with annexin V/PI and then detected by flow cytometry to obtain the flow cytometry diagram of the group activated with TGF-β1 as shown in FIG. 46 and flow cytometry diagram of the experimental group as shown in FIG. 47. Referring to FIGS. 46 and 47, the second anti-hepatic fibrosis formulation induced HSC apoptosis.

### Examples 2-5

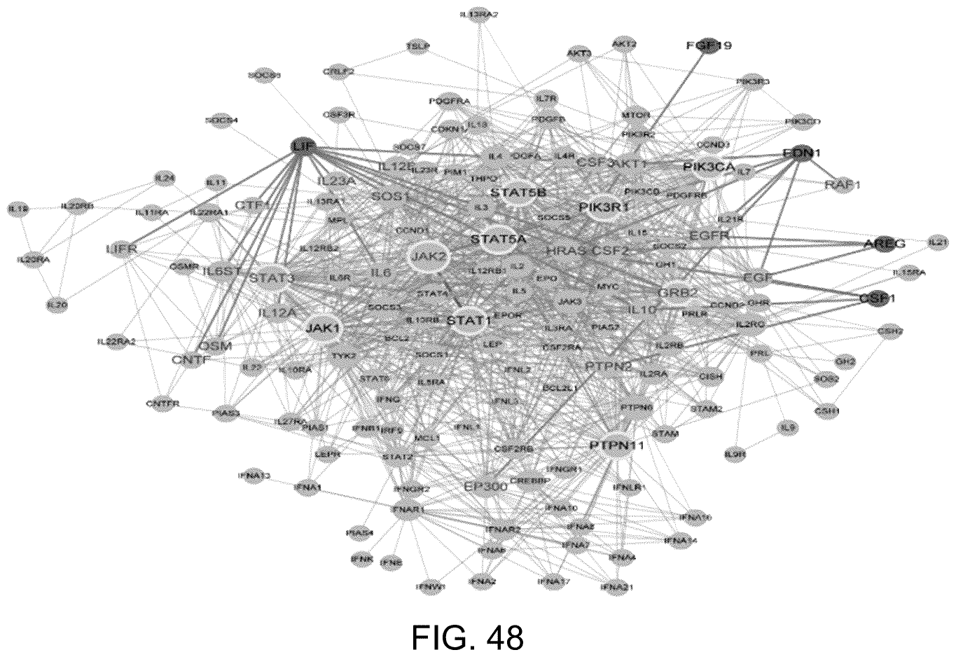

The JAK/STAT pathway played an important regulatory role in the progress of forming liver fibrosis. In this example, the first anti-hepatic fibrosis formulation of Example 1 obtained by in vitro culture of human liver precursor-like cells Human-HepLPCs were analyzed for proteomic composition by using a tandem mass spectrometry tag (TMT), so to obtain a schematic diagram of a visualized network between a JAK-STAT pathway in the first anti-hepatic fibrosis formulation and proteins involved in growth factor activity, cytokine activity and receptor-ligand activity in a first anti-hepatic fibrosis formulation as shown in FIG. 48 through analysis and construction of protein-protein interaction (PPI);

Referring to FIG. 48, the leukemia inhibitory factor (LIF), the endothelin 1 (EDN1), the colony-stimulating factor 1 (CSF1), the amphiregulin (AREG), the fibroblast growth factor 19 (FGF19) interacted with the intermediate molecules of the JAK-STAT pathway directly or indirectly.

### Examples 2-6

This example provided a third anti-hepatic fibrosis formulation including a recombinant human FGF19 (rhFGF19) and a recombinant human AREG (rhAREG). In this example, the third anti-hepatic fibrosis formulation was co-cultured with LX-2 to form a TGF-β1+rhFGF19+rhAREG group, so as to investigate its effect on LX-2, by referring to the method of Example 2-2, except that: the concentrations of rhFGF19 were 0.1 ng/mL, 1 ng/mL, 10 ng/mL, 100 ng/mL and 1000 ng/mL, respectively, and the concentrations of rhAREG were 0.1 ng/mL, 1 ng/mL, 10 ng/mL, 100 ng/mL and 1000 ng/mL, respectively. The rhFGF19 concentration and the rhAREG concentration were different in different experimental groups.

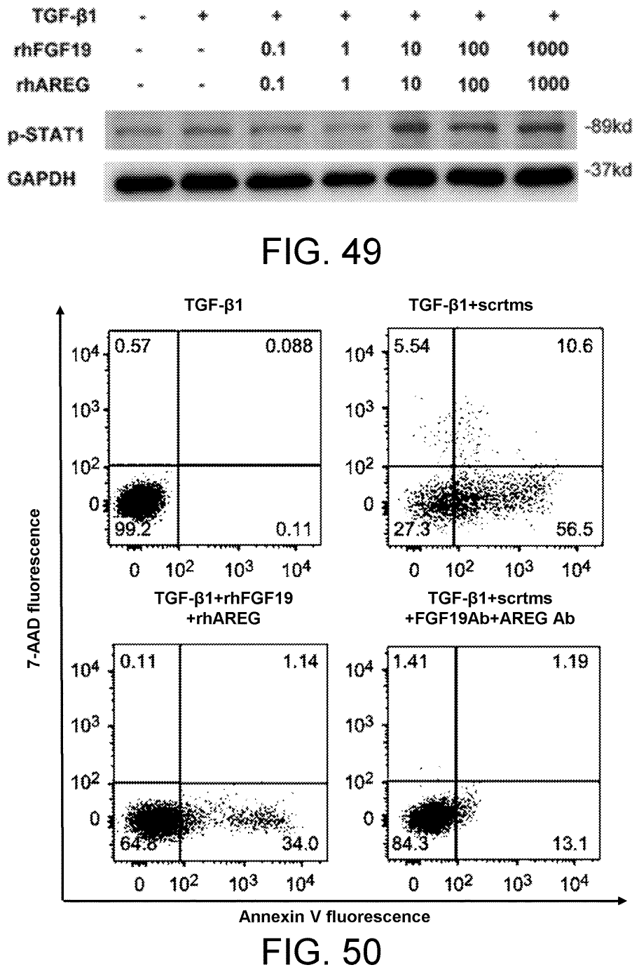

It was well known that p-STAT1 played an important role in the process of liver fibrosis, and mainly realized the anti-fibrosis effect by inhibiting the function of hepatic stellate cells. In this example, the control group, the group activated with TGF-β1 and different experimental groups were subjected to Western blot analysis, to obtain the comparison chart of the expression of a pSTAT1 signal in the cells of each group as shown in FIG. 49. Referring to FIG. 49, it could be seen that the level of p-STAT1 was increased when the concentration of each recombinant protein was not lower than 10 ng/mL.

### Examples 2-7

In this example, a FGF19 neutralizing antibody FGF19 Ab and an AREG neutralizing antibody AREG Ab were added to the system of co-culturing the first anti-hepatic fibrosis formulation with the activated LX-2 in Example 2-2 to form a TGF-β1+scrtms+FGF19 Ab+AREG Ab group, which were used for investigating the synergistic effect of rhFGF19 and rhAREG, together with the TGF-β1+rhFGF19+rhAREG group of Example 2-6 and the group activated with TGF-β1 and experimental group of Example 2-2. The concentrations of rhFGF19 and rhFGF19 in the co-culture system were both 100 nanograms/milliliter, and the concentrations of the FGF19 neutralizing antibody FGF19 Ab and the AREG neutralizing antibody AREG Ab were both 10 micrograms/milliliter in the co-culture system.

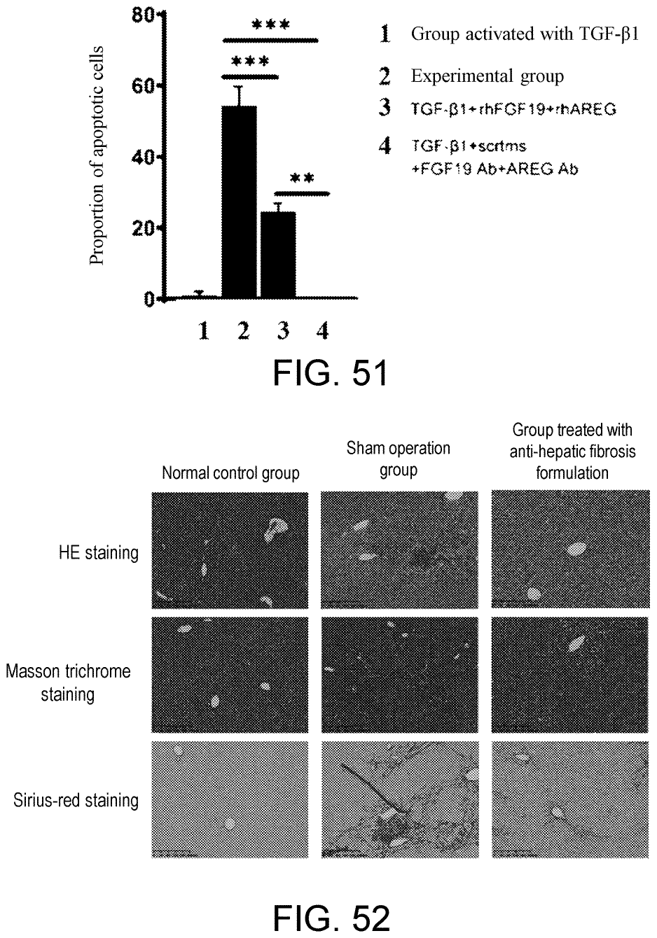

After 48 hours of co-culture, the cells in each group were analyzed by flow cytometry, so as to obtain a comparison chart of cell counts in each group as shown in FIG. 50. The number percentage of apoptotic cells was counted according to FIG. 50, so as to obtain a comparison chart of the number percentage of apoptotic cells in each group as shown in FIG. 51. Referring to FIGS. 50 and 51, in the presence of TGF-β1, compared with the co-culture system of TGF-β1 and LX-2, the combined use of rhFGF19 and rhAREG induced the apoptosis of LX-2 to a certain extent; and the combined use of rhFGF19 Ab and rhAREG Ab reduced the aforementioned LX-2 apoptosis-promoting effect, indicating that the combined use of rhFGF19 and rhAREG facilitated induction of STAT1-mediated HSC apoptosis.

### Examples 2-8

This example provided the use of the anti-hepatic fibrosis formulation in the preparation of an anti-hepatic fibrosis drug.

First, in order to induce liver fibrosis, thioacetamide (TAA) was used for inducing liver fibrosis. Particularly, a liver fibrosis model was formed by inducing 5-6 week-old female mice (C57BL/C) with thioacetamide (TAA), wherein TAA was diluted in physiological saline and injected intraperitoneally into the mice at a dose of 200 mg/kg for 7 weeks in total, with 3 times per week. There were three groups of animals, i.e. a normal control group, a sham operation group (group injected with PBS) and a group intervened with an anti-hepatic fibrosis formulation, and the number of animals of them were: 8, 8 and 8, respectively.

The secretory supernatant of Example 1 as the anti-hepatic fibrosis formulation was diluted with PBS to a total protein concentration of 2 mg/ml, so as to obtain an injection of anti-hepatic fibrosis formulation. After 7 weeks of TAA injection, the normal control group received no treatment, the sham operation group was injected with 250 ul of a PBS solution through the spleen, and the group intervened with an anti-hepatic fibrosis formulation was injected with 250 ul of the injection of anti-hepatic fibrosis formulation through the spleen. On 7 days after completion of the injection, the livers of the mice were taken, soaked in formalin solution for fixation, embedded and sectioned for detection by HE staining, Masson trichrome staining and sirius-red staining to comprehensively analyze the degree of liver fibrosis in the mice.

As shown in FIG. 52, after 7 weeks of TAA drug induction, the surfaces of the livers of the mice in the group injected with PBS was uneven and had a rough texture. Immunomorphology showed that collagen was widely present, and the interconnection between fibers separated the normal liver tissues. However, after treatment with the anti-hepatic fibrosis formulation, the texture of the livers of the mice in the group intervened with an anti-hepatic fibrosis formulation was closer to that of the normal group. Immunomorphology showed that the fibrous tissue was obviously reduced in the liver, and the fibrous tissues showed a slender structure, which suggested that the intervention with the anti-hepatic fibrosis formulation significantly improved the degree of liver fibrosis induced by TAA in mice.

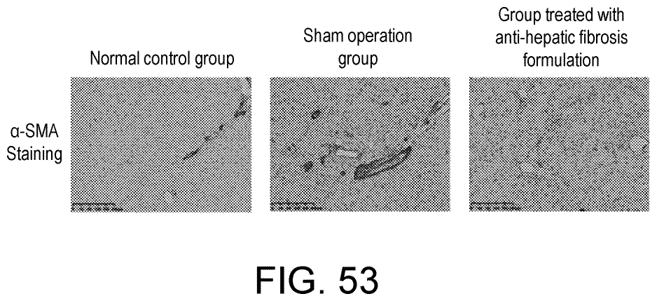

As shown FIG. 53, after 7 weeks of TAA drug induction, the number of activated hepatic stellate cells (α-SMA positive cells) in the livers of rats injected with PBS was significantly increased, while in the group intervened with an anti-hepatic fibrosis formulation, the number of the activated hepatic stellate cells (α-SMA positive cells) was significantly reduced compared with that of the sham operation group, which indicated that the anti-hepatic fibrosis formulation could inhibit the activation of hepatic stellate cells in vivo.

A qPCR test process of the example of the present invention was as follows: total mRNA was extracted by using an Eastep Super RNA extraction kit (Cat. No. LS1040, available from Promega). Reverse transcription was carried out by using a HiScript® II first-strand cDNA synthesis kit (Cat. No. R211-01, available from Vazyme). Then, real-time PCR was developed by using AceQ qPCR SYBR Green Master Mix (Cat. No. Q131-02, available from Vazyme) and a Life Technology ABI 7500 system. GAPDH expression was used as an internal control to determine a threshold cycle (CT), and the relative change in gene expression was calculated by using a Δ (ΔCT) method. In the example of the present invention, a transmission electron microscope (model Jem 1200ex II, available from JEOL) was used for observing the morphology of the cells. The cells were fixed with 2.5% glutaraldehyde and 2% osmic acid, then dehydrated and embedded in an epoxy resin, cut into 80-nm thick sections, and then observed after double staining with uranyl acetate and lead acetate.

For RNA sequencing and bioinformatics analysis of the example of the present invention, the total RNA was isolated from liver tissues by using a TRIzol® reagent; the genomic DNA was removed by using a DNase I (available from TaKara); and the concentration and purity of the RNA sample were determined by a 2100 bioanalyzer (available from Agilent) and quantified by ND-2000. Library preparation and Illumina Hiseq xten/Nova seq 6000 sequencing RNA seq transcriptome library was prepared according to a TruSeq™ RNA sample preparation kit available from Illumina, by using 1 g of total RNA; according to the instructions of the library construction protocol of Illumina, the fragmented RNAs were subjected to synthesis of cDNAs of the first strand and the second strand, followed by ligation with a linker and enrichment at low cycles; and after quantification, the paired-terminal RNA-seq sequencing library was sequenced at Guangzhou RiboBio Co., Ltd. by using an Illumina HiSeq xten/NovaSeq 6000 sequencer.

Gene expression of the example of the present invention was normalized by EDASEQ. Differentially expressed genes were obtained by using DESeq2 of version 1.10.1, and a cut-off value of an Q value<0.05 and log 2 (fold of change)>1 were used for identifying the differentially expressed genes. All differentially expressed mRNAs were selected for G0 analysis with clusterProfiler. glbase was used for other analysis.

In the example of the present invention, the total protein of cells or secretinites were extracted by using a RIPA buffer (P0013B, available from Beyotime) containing a protease inhibitor mixture (P1010, available from Beyotime). The samples were sonicated for 30 s on ice and then centrifuged at 12,000×g at 4° C. for 15 minutes. The supernatant was collected and quantified with a BCA protein analysis kit (ZJ101, available from Epizyme). The quantitative protein samples were resolved by 5×SDS-PAGE (P0015, available from Beyotime) and transferred to a hydrophobic PVDF transfer membrane (IPVH00010, available from Merck Millipore). The membrane was blocked in 5% BSA in TBST for 1.5 hours, and incubated with an primary antibody at 4° C. overnight. Then the membrane was washed with TBST for three times, and incubated with a secondary antibody at room temperature for 2 hours. Imprints were detected by using an enhanced ECL chemiluminescent detection kit (E411-04, available from Vazyme) and a digital luminescent image analyzer (BioRad). Densitometric analysis of each wave band was determined by using ImageJ software or QingXiang software. The primary and secondary antibodies as used were listed in Table 1.

TABLE 1

| ID | Description | GeneRatio | BgRatio | p. adjust | Count | Enrich_factor* |
|---|---|---|---|---|---|---|
| GO:0071674 | mononuclear cell migration | 7/25 | 68/17913 | 4.45E−10 | 7 | 73.7594 |
| GO:0046427 | positive regulation of JAK- | 7/25 | 85/17913 | 1.84E−09 | 7 | 59.0075 |
| | STAT cascade | | | | | |
| GO:1904894 | positive regulation of STAT | 7/25 | 88/17913 | 2.23E−09 | 7 | 56.9959 |
| | cascade | | | | | |
| GO:0002687 | positive regulation of | 9/25 | 121/17913 | 1.11E−11 | 9 | 53.2949 |
| | leukocyte migration | | | | | |
| GO:0050921 | positive regulation of | 8/25 | 130/17913 | 6.24E−10 | 8 | 44.0935 |
| | chemotaxis | | | | | |
| GO:0030595 | leukocyte chemotaxis | 11/25 | 188/17913 | 8.23E−13 | 11 | 41.9240 |
| GO:0046425 | regulation of JAK-STAT | 7/25 | 120/17913 | 1.52E−08 | 7 | 41.7970 |
| | cascade | | | | | |
| GO:0050731 | positive regulation of | 10/25 | 184/17913 | 9.68E−12 | 10 | 38.9413 |
| | peptidyl-tyrosine | | | | | |
| | phosphorylation | | | | | |
| GO:1904892 | regulation of STAT cascade | 7/25 | 129/17913 | 2.17E−08 | 7 | 38.8809 |
| GO:0097529 | myeloid leukocyte migration | 9/25 | 170/17913 | 1.49784E−10 | 9 | 37.9334 |

In the example of the present invention, an annexin V-FITC apoptosis detection kit available from Beyotime and an annexin V-APC apoptosis detection kit available from Biogems were used for detecting apoptosis in LX-2 through annexin V/propidium iodide (PI) or annexin V/7-AAD. Particularly, the cells were collected and resuspended in a binding buffer, and then stained with annexins V and PI or 7-AAD according to the instructions of the kit. 0.5 μM of a protein kinase inhibitor Staurosporine was used as a apoptosis-promoting control (positive control). Fluorescence was detected by a BD-facverse flow cytometer, and data analysis was conducted with FlowJo software.

In the example of the present invention, a cytokine antibody array (AAH-INF-G3, RayBio® G series) was used for measuring the expression of 40 cytokines in the culture supernatant. Positive signals were detected with a laser scanner. The basic statistic data used for significance analysis was the fold of change. Differentially expressed proteins (DEP) were defined as proteins with a fold of change greater than 1.2 or less than 0.83 (absolute log fc>0.263). The functions of cytokines were annotated by gene ontology (G0) annotations.

Raw mass spectrometry (MS) data files were processed by using proteome discovery (PD) software (version 2.4.0.305) and a built-in Sequest HT search engine. The MS spectrum list was searched according to UniProt FASTA database of Homo sapiens (UniProt-Human-9606-2020-10.FASTA), wherein aminomethyl [C], a TMT 6 complex (K) and a TMT 6 complex (N-terminal) were used as fixed modifications, and oxidation (M) and acetyl (N-terminal of the protein) were variable modifications. The parameters used for identifying peptides were: 10 ppm precursor ion mass tolerance, 0.02 Da fragment mass tolerance, an up to 2 times of deletion cleavage. The false discovery rates (FDRs) of PSM and a peptide level were both set to 0.01. The functions of the proteins were annotated by gene ontology (G0) annotations (http://www.geneontology.org/). The Kyoto Encyclopedia of Genes and Genomes (KEGG) database was used for analyzing an enriching pathway. A two-tailed Fisher's exact test was used in the G0-KEGG enrichment analysis. P<0.05 was considered significant. Cytoscape version 2.6 (www.Cytoscape.org) was used for visualizing and analyzing an interaction network of molecules and proteins. Differentially expressed proteins were ranked by hierarchical clustering and represented by a heatmap. The heatmap was generated by R software (http://www.r-project.org).

### Examples 2-9

In this example, the HepLPCs obtained in Example 2-1 were used as the main component of a first cell preparation. The first cell preparation was co-cultured with the human immortalized hepatic stellate cell line LX-2 to investigate the death-promoting effect of the cell preparation on LX-2. LX-2 in this example was purchased from Procell.

For the convenience of experimental observation, first Human-HepLPCs and LX-2 were stained with lipophilic membrane dyes DiO and DiI respectively, and then the stained cells were resuspended in a DMEM complete medium to obtain different cell preparations. Specifically, Human-HepLPCs obtained by in vitro culture in a TEM medium in Example 2-1 were digested and washed, then resuspended in a serum-free DMEM complete medium containing 5 uM of DiO (available from Beyotime) or DiI (available from Beyotime) respectively, incubated in an incubator for 10 minutes, and then washed with a PBS buffer to remove unbound dyes, and finally the stained cells were resuspended in a DMEM complete medium to obtain the labelled cell preparation as used in Example 3. after entering the cell membrane, DiO could diffuse laterally to gradually stain the cell membrane of the entire cell in green; and after entering the cell membrane, DiI could diffuse laterally to gradually stain the cell membrane of the entire cell in red.

In Example 2-9, a cell preparation containing the DiI-labeled LX-2 and a cell preparation containing DiO-labeled HepLPCs were mixed according to a proportion of 1:1, then added with 2.5 ng/ml of TGF-β, and then inoculated into a 96-well plate with low adhesion according to 3,000 cells/well to serve as a co-culture group; the cell preparation containing the DiI-labeled LX-2 was inoculated into a 96-well plate with low adhesion according to 3,000 cells/well to serve as a LX-2 group; and the cell preparation containing the DiI-labeled LX-2 was added with 2.5 ng/ml of TGF-β, and then inoculated into a 96-well plate with low adhesion according to 3,000 cells/well to serve as a group added with LX-2. After 48 hours of co-culture of the LX-2 group, the group added with LX-2 and the co-culture group, the culture medium was discarded by pipetting, and after the cells were observed and photographed under a microscope, the RNAs of the LX-2 group and the group added with LX-2 were collected respectively. For the co-culture group, LX2 cells with red DiI fluorescence label were sorted by flow cytometry, and then the RNA of the cells was collected.

The total RNAs of LX2 in the 3 groups was extracted by employing an RNA extraction kit (purchased from Promaga); and PCR was conducted on a PCR instrument (purchased from Roche) with a SYBR Green PCR kit (purchased from Vazyme). Verified genes included collagen-related genes COL3A1 and COL1A1; and fibrosis-related genes α-SMA, Vimentin, and Timp1.

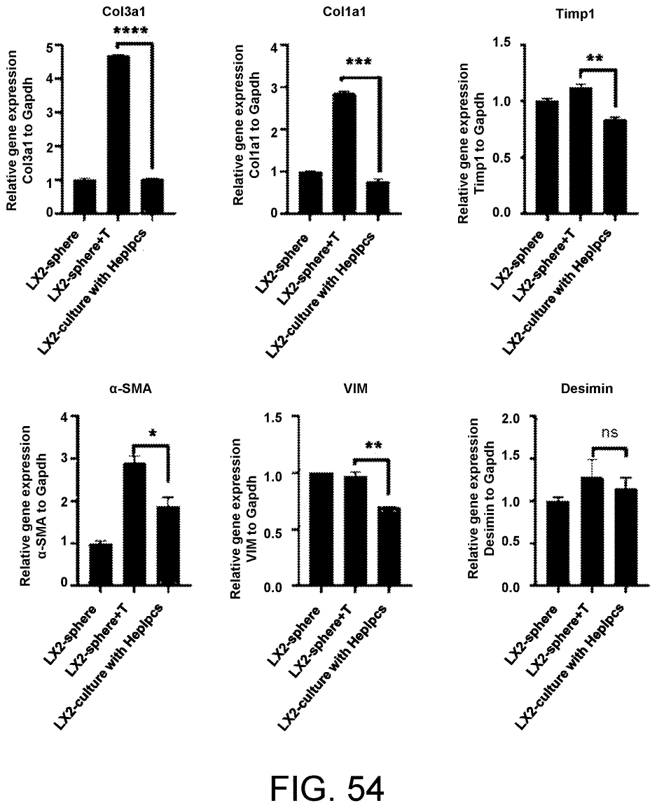

In this example, after 48 hours of co-culture of the LX-2 group, the group added with LX-2 and the co-culture group, the LX-2 cells in each group were determined for the collagen-related genes and fibrosis-related genes by quantitative fluorescence PCR (real-time PCR), so as to obtain a comparison chart of the expression of the collagen-related genes and the fibrosis-related genes of the LX-2 cells in each group as shown in FIG. 54. Referring to FIG. 54, in hepatic stellate cells activated by TGF-β1, the levels of the fibrosis-related genes COL3A1, COL1A1, α-SMA, Vimentin, Timp1 and Desimin were all significantly up-regulated, while after the hepatic stellate cells activated by TGF-β1 were co-cultured with liver precursor cells, the aforementioned genes were all significantly down-regulated, which proved that the liver precursor cells could significantly inhibit the activation of the hepatic stellate cells in vitro.

### Examples 2-10

This example provided the use of a second cell preparation prepared from the Human-HepLPCs of Example 2-1 as liver-derived cells with precursor characteristics in the preparation of an anti-hepatic fibrosis drug.

The second cell preparation was a cell suspension obtained by resuspending the Human-HepLPCs in PBS. Specifically, 5×10⁶ Human-HepLPCs were resuspended in 500 μl of PBS.

The in vitro organ-like model of liver fibrosis was a mammalian liver cirrhosis model induced by thioacetamide (TAA). The modeling method was as follows: 5-6 week-old female Sprague-Dawley rats (available from Vitalriver) were induced with TAA diluted with physiological saline at an injection dose of 200 mg/kg every week, twice a week for an induction time of 13 weeks to complete modeling.

In this example: 8 successfully modeled rats were subjected to any treatment to serve as a normal group; 8 successfully modeled rats were injected with 500 μl of PBS through spleens to serve as a sham operation group; taking the first time of induction as an initial time node, 8 successfully modeled rats were injected with an immunosuppressive drug tacrolimus (FK506) at an injection dose of 0.2 mg/kg through spleens every day at the 13th and 15th weeks, and started to be injected with the second cell preparation twice per week on the day after the first injection of FK506. The rats were sacrificed on the third day of week 17.

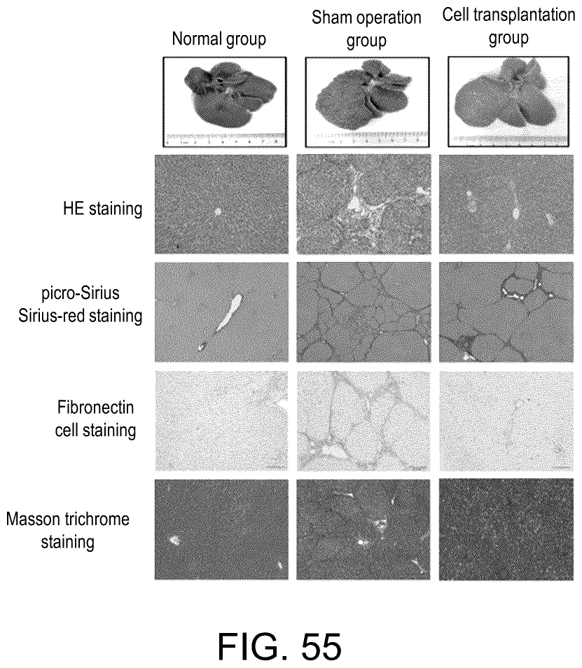

In this example, the livers of rats in each experimental group were taken for further staining analysis, hydroxyproline content analysis, and liver fibrosis scoring. Specifically referring to FIGS. 55 to 60, it could be seen that the injection of the second cell preparation could reduce the accumulation of extracellular matrix (ECM), reduce the hydroxyproline level of the rats, and relieve liver cirrhosis in the rats.

FIG. 55 was a comparison chart of photographs obtained after H&E staining, picro-Sirius sirus red staining, Masson trichrome staining and fibronectin (FN) immunostaining of liver tissues taken from the normal group, the sham operation group and the cell transplantation group, wherein the scale bar in the figure was 100 μm.

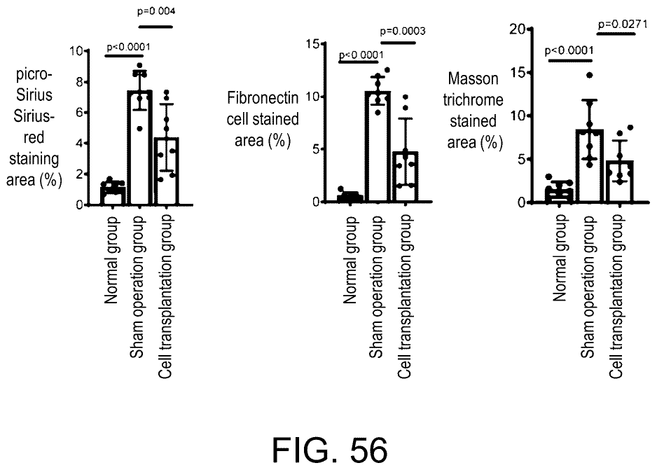

FIG. 56 was a comparison chart of the relative quantification of liver fibrosis regions and fibronectin positive-stained regions from statistics after picro-Sirius sirus red staining, Masson trichrome staining and fibronectin (FN) immunostaining of liver tissues taken from the normal group, the sham operation group and the cell transplantation group.

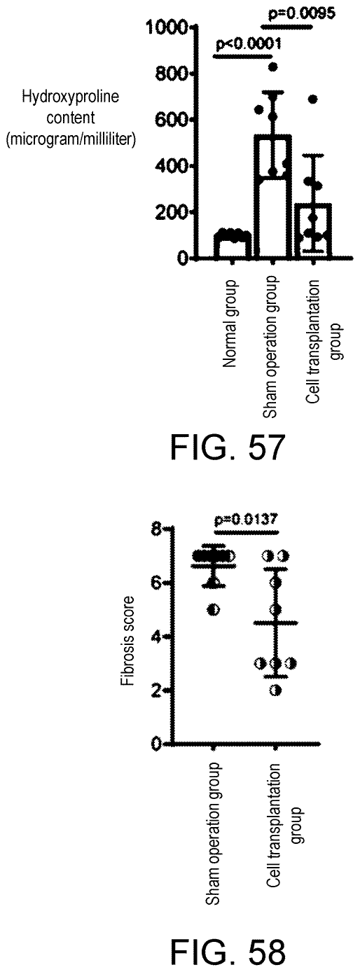

FIG. 57 showed the analysis results of the determination of hydroxyproline content in a normal group, a sham operation group and a cell transplantation group. FIG. 58 showed the analysis results of liver fibrosis scoring of liver tissues taken from the sham operation group and the cell transplantation group.

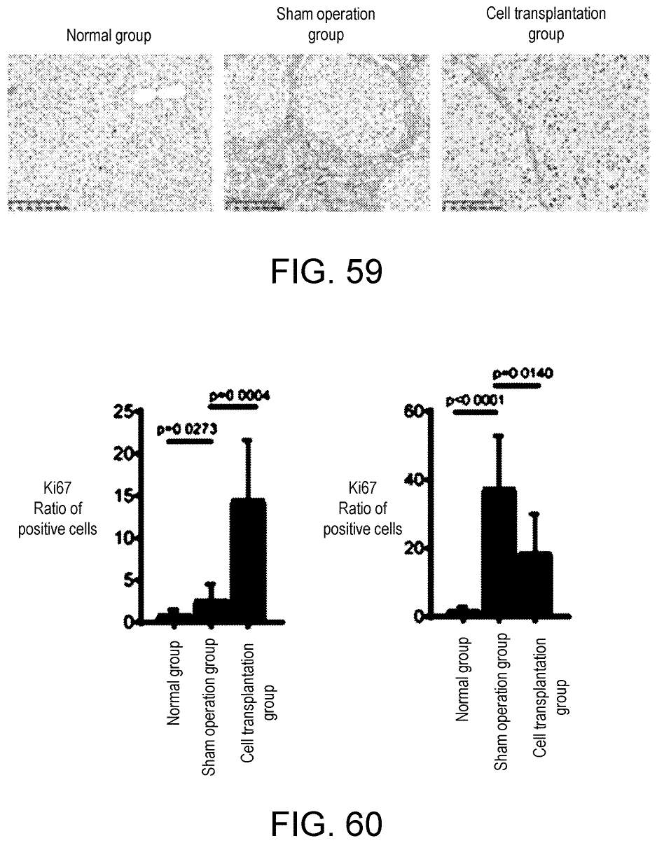

FIGS. 59 and 60 were respectively a comparison chart of photographs obtained by Ki67 immunohistochemical staining of liver tissues taken from the normal group, the sham operation group and the cell transplantation group, and a quantification result obtained by counting the positively stained cells according to the photographs as shown in FIG. 59.

### Examples 2-11

This example provided the use of a third cell preparation prepared from the Rat-HepLPCs of Example 2-2 as liver-derived cells with precursor characteristics in the preparation of an anti-hepatic fibrosis drug.

The difference between the third cell preparation in this example and the second cell preparation in Example 2-4 was that the liver-derived cells were Rat-Hep LPCs.

The in vitro organ-like model of liver fibrosis was a carbon tetrachloride-induced mammalian liver cirrhosis model. The modeling method was as follows: 5-6 week-old female Sprague-Dawley rats (available from Vitalriver) were induced with a mixed injection of CCl4 and olive oil having a CCl4 content of 40% at an injection dose of 1 mL/kg every week for an induction time of 13 weeks to complete the modeling.

The treatment of the normal group and the sham operation group of this example were consistent with those of Example 4, and the difference between the treatment of the cell transplantation group and that of Example 2-4 was that: intervention with the immunosuppressive drug tacrolimus was not used, and the rats were injected with the third cell preparation at week 13 and then sacrificed at week 17.

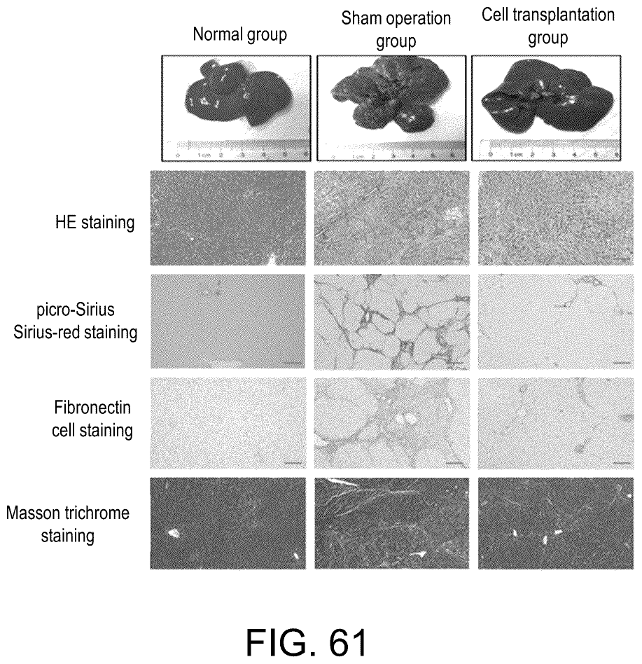

In this example, livers and blood were taken from rats in each group for further staining analysis, hydroxyproline content analysis, and liver fibrosis scoring. Specifically referring to FIGS. 61 to 65, it could be seen that injection of the second cell preparation could reduce the accumulation of extracellular matrix (ECM), reduce the hydroxyproline level in the rats, inhibit the production of the fibrotic cytokine TGF-β and its intracellular signaling molecules in fibrotic tissues, and relieve liver cirrhosis in the rats.

FIG. 61 was a comparison chart of photographs obtained after H&E staining, picro-Sirius sirus red staining, Masson trichrome staining and fibronectin (FN) immunostaining of liver tissues taken from the normal group, the sham operation group and the cell transplantation group, wherein the scale bar in the figure was 100 μm.

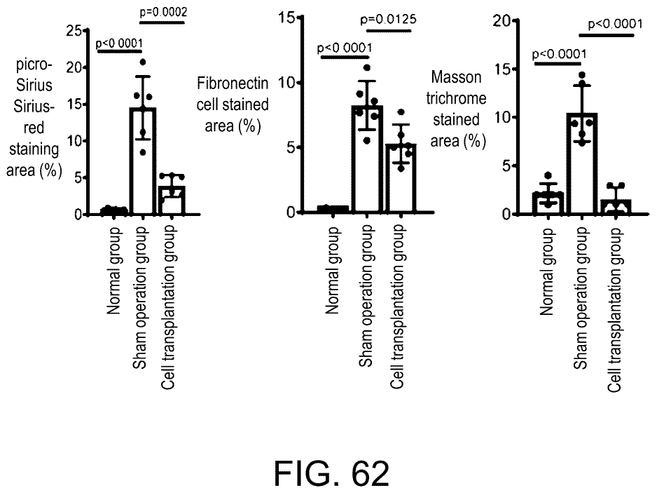

FIG. 62 was a comparison chart of the relative quantification of liver fibrosis regions and fibronectin positive-stained regions from statistics after picro-Sirius sirus red staining, Masson trichrome staining and fibronectin (FN) immunostaining of liver tissues taken from the normal group, the sham operation group and the cell transplantation group.

FIG. 63 showed the analysis results of the determination of hydroxyproline content in the liver tissues taken from the normal group, the sham operation group and the cell transplantation group. FIG. 64 showed the analysis results of liver fibrosis scoring of liver tissues taken from the sham operation group and the cell transplantation group.

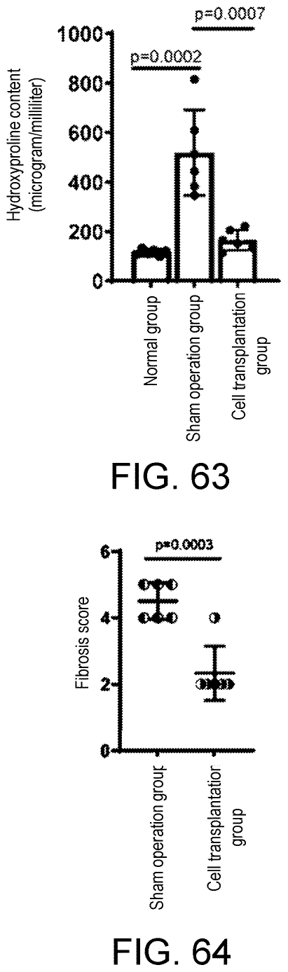

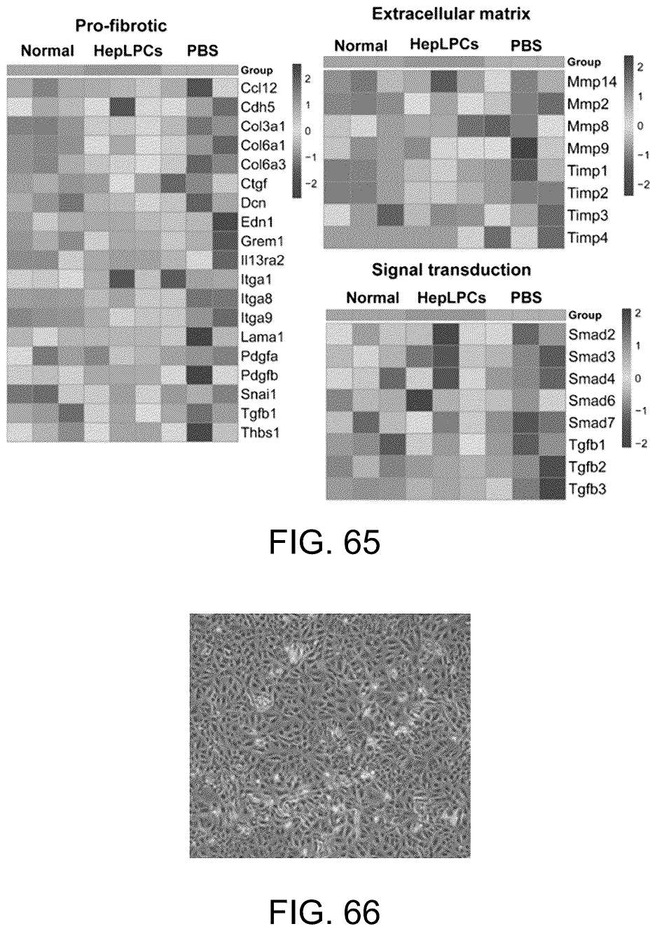

FIG. 65 was a heat map obtained after analyzing the gene expression levels related to the occurrence of liver fibrosis related to fibrosis promoting, extracellular matrix, and signal transduction of blood in the normal group, the sham operation group and the cell transplantation group.

In the example of the present invention:

Liver tissues were fixed in 4% paraformaldehyde (PFA), embedded in paraffin, and subsequently cut into 4 μm-thick sections. The liver sections were routinely stained for collagen deposition with hematoxylin and eosin (H&E) staining, picro-Sirius sirius red staining, and Masson's trichrome (MT) staining. The quantification of collagen types I and III was analyzed with a microscope (Olympus BX50) under polarized light. Using a polarized filter, in sirius red-stained sections, the type I collagen fibers would be presented as orange to red, and the type III collagen fibers would be presented as yellow to green. The sections stained with sirius red were used for liver fibrosis scoring under an Ishak scoring system. For immunohistochemistry (IHC), tissue sections were stained with primary antibodies against fibronectin, GFP, KI67, HLA class I, CK18 and ALB. The liver fibrosis score and Ki67+ cells of two independent individuals were calculated by a double-blind method, and the mean value of the results was used for analysis. Representative images of H&E, picro sirius red, MT staining and IHC were photographed at Turbo with Aperio available from Leica. Quantification was performed by dividing the positively stained area by a total sampled area using Image J software.

Immunofluorescence was performed on liver sections (5 μm=thick) or cell spheroids in a cryostat. Prior to antibody staining, the liver sections or cell spheroids were fixed in 4% paraformaldehyde (PFA), then permeabilized with 0.3% Triton X-100 and blocked with 3% bovine serum albumin (BSA), and then incubated to obtain a primary antibody against α-SMA that cleaves caspase 3 at 4° C. overnight (see Supplementary Table 1 for details), and the cell spheroids were cultured at 4° C. overnight to obtain primary antibodies against ALB, CYP3A4, TTR, CK19, SOX9 and AFP (see Supplementary Table 1 for details). The samples were then washed with PBS and stained with a fluorescently labeled secondary antibody. The control samples were similarly treated without incubation with the primary antibody. Representative images of immunofluorescent staining were taken with a confocal microscope (TCS SP8, available from Leica).

An hydroxyproline assay was performed according to the protocol of the manufacturer (Solarbio, BC0250). Briefly, liver tissues (200 mg) were homogenized in an extracting solution, boiled in an oven at 110° C. for 2 to 6 hours until there was no visible big mass, and then centrifuged at 16,000 rpm for 20 minutes, and the pH value was adjusted to 6 to 8 with 10 mol/L of NaOH (about 1 ml). The volume of the hydrolyzed sample was set to 4 ml with distilled water, the supernatant was transferred onto a 96-well plate, and measured following the protocol of the manufacturer.

In the following examples, a hepatic macrophage regulator was prepared as a liver disease regulatory formulation, and the use of this liver disease regulatory formulation was investigated. The details were as follows:

### Examples 3-1

This example provided a first hepatic macrophage regulator, the preparation method of which was as follows:
S0: human primary hepatocytes were provided;
S1: the primary hepatocytes were cultured by using a hepatocyte proliferation medium (abbreviated as a TEM medium) until the confluence was no less than 80%;
S2: the cells obtained after the step S1 were digested and then subcultured according to a proportion of 1:3;
S3: the TEM medium was replaced with a high-glucose serum-free DMEM medium, and then in vitro culture was continued for 24 hours; and
S4: after the in vitro culture was completed, the cell supernatant was collected and centrifuged, the cell debris was removed to obtain a culture supernatant, and the culture supernatant was used as the first hepatic macrophage regulator.

In the step S0 of this example, the human primary hepatocytes were available from Research Institute for Liver Diseases (Shanghai) Co. Ltd. Specifically, before the TEM culture, the human primary hepatocytes were subjected to Percoll density gradient centrifugation combined with flow sorting to exclude CD24-positive and EpCAM-positive precursor cells.

In the TEM medium of the step S1 of this example, based on the content in the HepX Basal medium, it consisted of 1% of a serum-free additive N2 (100×), 1% of a serum-free additive B27 (50×), 20 ng/mL of HGF, 20 ng/mL of EGF, 20 ng/mL of FGF, 1.25 μM of N-acetyl-L-cysteine, 10 μg/mL of ascorbic acid, 1 μM of a TGF-β signal inhibitor A8301, 3 μM of a Wnt signaling pathway agonist CHIR99021, 10 μM of a ROCK kinase inhibitor Y27632, 2% of a penicillin-streptomycin double antibody (100×) and 10% of fetal bovine serum FBS. Among them: the HepX Basal medium, N2, B27, and the penicillin-streptomycin double antibody were available from Shanghai Basalmedia Technologies Co., Ltd.; the fetal bovine serum FBS was available from Biological Industries, Israel; HGF was available from Sino Biological, USA; EGF and FGF were available from Peprotech, USA; A8301, CHIR99021 and Y27632 were available from Topscience, Shanghai.

In the step S1, the step of culturing the primary hepatocytes included: resuspending the human primary hepatocytes in a TEM medium and then inoculating them into a 6-well plate coated with Vitronectin XF™ (available from STEMCELL Technologies Inc., Canada) at a density of 0.5-1×10⁵/cm² for culture. During the culture process, the TEM medium was replaced every 2-3 days.

In the step S2, the step of digesting the cells obtained after the step S1 and then subculturing according to a proportion of 1:3 included: digesting with 0.25% Trypsin-EDTA (available from Gibco, USA), inoculating into a new culture dish according to a proportion of 1:3, and conducting expansion culture to the third passage until the cell confluency reached 80%.

In the step S4, the cell supernatant was centrifuged under a centrifugal force of 300 g for 10 minutes to remove cell debris, so as to obtain the first hepatic macrophage regulator.

In this example, the human liver precursor-like cells (HepLPCs) obtained through the step S2 were identified and analyzed through flow cytometry. For the comparison chart of the expression of various liver precursor-related markers in HepLPCs cells, please refer to FIG. 34. Referring to FIG. 34, it could be seen that the HepLPCs expressed the liver precursor-related markers CK19 and CD24 and the hepatocyte-like marker ALB, showing the characteristics of liver precursor cells. The expression levels of the hematopoietic stem cell antigen CD34 and the leukocyte common antigen CD45 were less than 2%, showing low immunogenicity.

In this example, the cells obtained after being cultured in a TEM medium for 10 days were photographed under a bright field, so as to obtain a bright-field photograph of the HepLPCs as shown in FIG. 66. Referring to FIG. 66, it could be seen that the HepLPCs cells were spindle-shaped and grown adherently, and the cells grown to fully cover the field of view and exhibited the characteristics of hepatic precursor cells with a high nuclear/cytoplasmic ratio.

### Examples 3-2

This example provided a modeling method for an inflammatory cell model, and the inflammatory cell model was co-cultured with the first hepatic macrophage regulator (abbreviated as HepLPCs-CM) of Example 1 to investigate the effect of the first hepatic macrophage regulator on type M1 macrophages.

In this example, the inflammatory cell model was established by stimulating macrophages with lipopolysaccharide LPS. The specific process included: mouse primary bone marrow-derived macrophages (BMDMs) were acquired, resuspended in a BMDM medium and inoculated, and then added with a mouse granulocyte-macrophage colony-stimulating factor GM-CSF available from Novoprotein to induce differentiation of BMDMs for a period of 7 days until cell maturity. The resultant mature primary macrophages were induced with orientational polarization in vitro by LPS for 6 hours, and then digested to obtain the inflammatory cell model. Among them, the concentration of LPS was controlled to be 100 ng/mL during the process of stimulation with LPS.

Specifically, 6-8-week-old adult C57 mice were sacrificed by spinal dislocation, and the bone marrow mass in a femoral myelocavity was taken; the bone marrow mass was filtered through a 70 μm cell sieve and centrifuged at 500 g for 5 minutes, and then the supernatant was discarded; the precipitate was resuspended with an erythrocyte lysis buffer (available from Shanghai beyotime Biotechnology Co., Ltd.) and centrifuged again at a centrifugal force of 500 g for 5 minutes, and the supernatant was discarded to obtain a secondary precipitate; and the secondary precipitate was repeatedly resuspended in a BMDM medium and centrifuged to fully wash off the residual erythrocyte lysis buffer and cell debris in the precipitate. Finally, the purified cells were resuspended in a BMDM medium, and then inoculated into a 12-well plate according to (8-10)×10⁵/well, wherein each well was added with mouse GM-CSF at a controlled concentration of 40 ng/mL. 6-8 hours after completion of inoculation, the cells were transferred into a new culture dish for culture, and the medium was replaced with a BMDM medium supplemented with 40 ng/mL of mouse GM-CSF once every 3 days until day 7, so as to achieve the differentiation and maturation of macrophages. The BMDM medium consisted of 500 mL of a1640 culture medium (available from Shanghai Basalmedia Technologies Co., Ltd.), 5% of a penicillin-streptomycin double antibody and 10% of FBS.

Specifically, after the macrophages were differentiated and matured, the culture medium was replaced with a BMDM medium containing 100 ng/mL of LPS, and culture was conducted for 6 hours to complete the induction by orientational polarization in vitro.

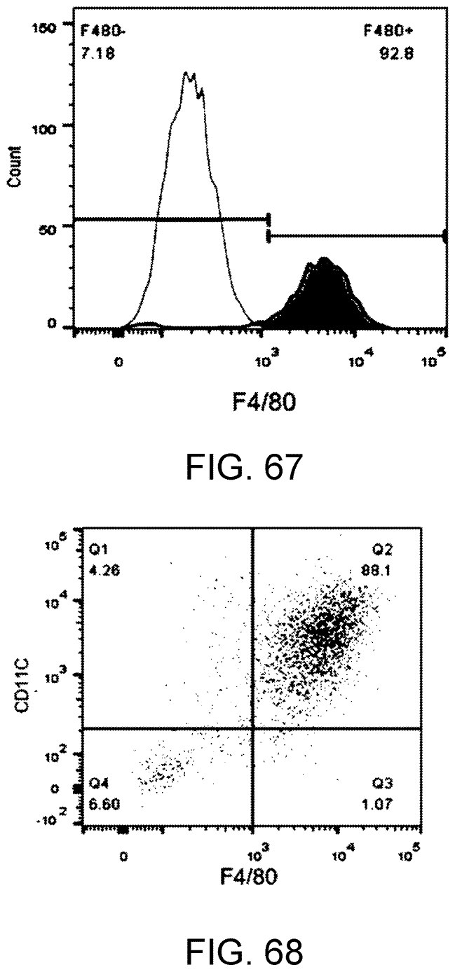

In this example, the BMDMs obtained by inducing with the mouse GM-CSF and the BMDMs obtained by inducing with LPS in vitro orientational polarization were subjected to cell identification by flow cytometry, so as to obtain the analysis result of the proportion of total macrophages as shown in FIG. 67 and the analysis result of the proportion of the type M1 macrophages as shown in FIG. 68. Referring to FIGS. 67 and 68, it could be seen that the expression of the total macrophage phenotype marker F4/80+ in the BMDMs obtained by inducing with the mouse GM-CSF accounted for 92.8%, that was, the total macrophage accounted for 92.8%, which was line with the experimental cell purity of BMDMs; and in the BMDMs obtained after LPS stimulation for 6 hours, the expression of M1 phenotype markers CD11c+ and F4/80+ accounted for 88.1%, that was, the type M1 macrophages accounted for 88.1%. It could be seen that in the inflammatory cell model obtained after the BMDMs were differentiated as induced by the mouse GM-CSF and LPS stimulation in this example, the proportion of the type M1 macrophages met the requirement of purity.

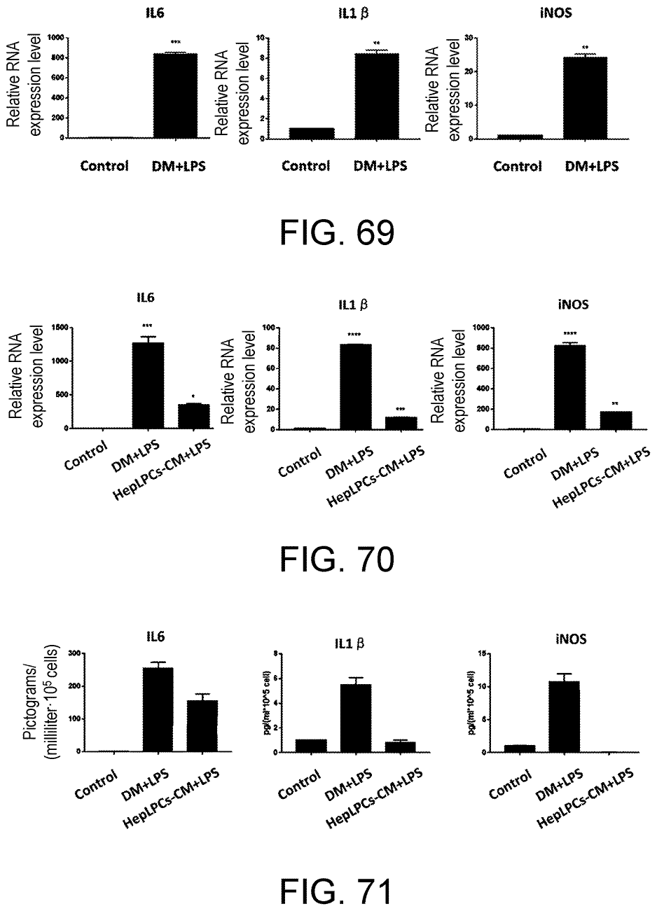

In this example, untreated BMDMs (the Control group) and BMDMs obtained by inducing through LPS in vitro orientational polarization (the DM+LPS group) were subjected to cell identification by flow cytometry, so as to obtain a comparison chart of M1-related inflammatory gene expression as shown in FIG. 69. Referring to FIG. 69, compared with the control group, the expression levels of M1-related genes in the BMDMs that had been stimulated by LPS for 6 hours were all up-regulated. Among them, the expression level of IL6 was up-regulated to 823.200±174.500, the expression level of IL1β was up-regulated to 8.389±0.029, and the expression level of iNOS was up-regulated to 24.650±1.196.

In this example, HepLPCs-CM was co-cultured with the aforementioned BMDMs obtained by inducing through LPS in vitro orientational polarization for 6 hours to obtain a HepLPCs-CM+LPS group, and the HepLPCs-CM+LPS group, the Control group and the DM+LPS group were subjected to RNA extraction and gene expression analysis to obtain a comparison chart of the expression of M1-related inflammatory genes in each group as shown FIG. 70. Referring to FIGS. 69 and 70, compared with the positive control treatment group DM+LPS, the expression of M1-related genes was significantly down-regulated after the BMDMs obtained by inducing through LPS in vitro orientational polarization were co-cultured with HepLPCs-CM. The expression level of IL6 was down-regulated to 346.300±20.810, the expression level of IL1β was down-regulated to 11.290±0.10, and the expression level of iNOS was down-regulated to 169.800±9.711.

In this example, the cell culture supernatants collected from the experimental group, the control group and the positive control treatment group were tested for cytokine concentration by using an ELISA kit available from MultiSciences (Lianke) Biotech Co., Ltd., so as to obtain a comparison chart of the expression levels of M1-related inflammatory genes in the cell culture supernatant of the HepLPCs-CM+LPS group, the Control group and the DM+LPS group as shown in FIG. 71. For specific operation methods, please refer to the instructions of the kit. For the collection method of the cell culture supernatant, please refer to Example 1, which would not be repeated here. Referring to FIG. 71, compared with the positive control treatment group, the secretion of inflammatory factors in the supernatant of the BMDMs obtained by inducing through LPS in vitro orientational polarization was decreased after being treated with HepLPCs-CM. Among them, the secreted IL6 was 138.700±32.130 pg/(mL*10⁵ cells), the secreted IL1β was 0.710±0.019 pg/(mL*10⁵ cells), and the secreted iNOS was 0.095±0.001 pg/(mL*10⁵ cells).

Currently, a large number of experimental and clinical data supported the central role of macrophages in the occurrence and development of nonalcoholic fatty liver disease (NAFLD) and nonalcoholic steatohepatitis (NASH). Under the induction with different factors, two classic cell subpopulations of the macrophages could occur, namely the type M1 macrophages and the type M2 macrophages. The functions of the two were completely different. It was generally believed in the industry that the type M1 macrophages promoted inflammatory responses by producing a large number of pro-inflammatory cytokines such as interleukin-1β (IL-1β), TNF-α, interleukin-6 (IL-6), as well as nitric oxide (NO) and reactive oxygen species (ROSs) to mediate the inflammatory response of a body.

From FIGS. 70 and 71 and the respective analysis results, it could be seen that the HepLPCs-CM could inhibit the inflammatory activation of macrophages as induced by LPS, and significantly reduce the expression of genes and secreted proteins of inflammation-related factors.

### Example 3-3

This example provided a modeling method for a repair cell model, and the repair cell model was co-cultured with the first hepatic macrophage regulator (abbreviated as HepLPCs-CM) of Example 1 to investigate the effect of the first hepatic macrophage regulator on type M2 macrophages.

In this example, the repair cell model was established by stimulating macrophages with the interleukin 4 (IL-4) available from Novoprotein. The specific process included that: for the method of acquiring mouse primary bone marrow-derived macrophages (BMDMs) and the method of conducting differentiation of the BMDMs with the mouse GM-CSF for 7 days, please refer to Example 2. The mature primary macrophages were induced by in vitro orientational polarization with IL-4 for 6 hours, and then the macrophages were digested to obtain the repair cell model. Among them, the concentration of IL-4 was controlled to be 40 ng/mL during the process of stimulation with IL-4. For other specific experimental steps, please refer to Example 3-2.

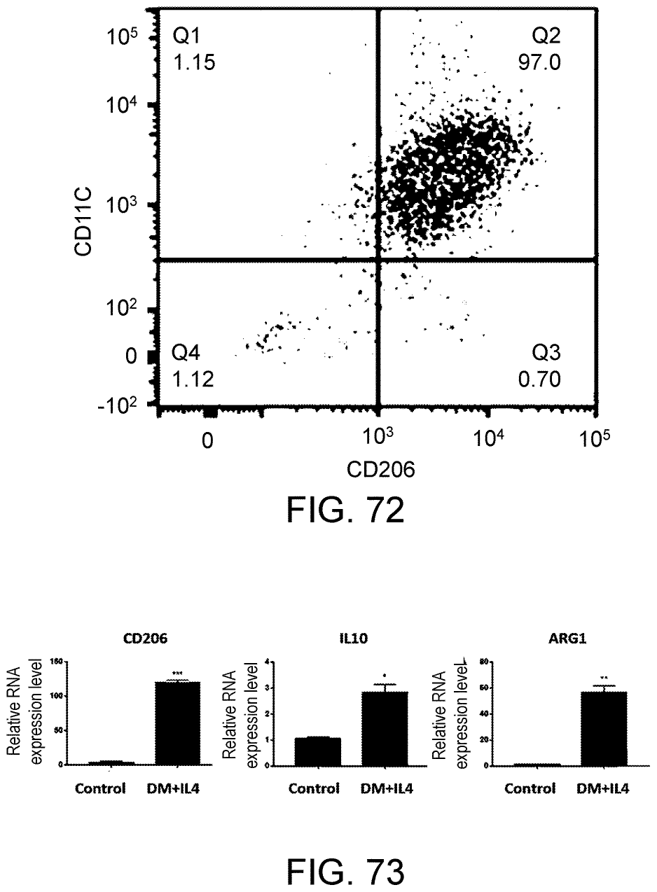

In this example, the BMDMs induced by IL-4 in vitro orientational polarization were subjected to cell identification by flow cytometry, so as to obtain the analysis result of the proportion of type M2 macrophages as shown in FIG. 72. Referring to FIG. 72, it could be seen that in the BMDMs obtained after IL-4 stimulation for 6 hours, the expression of the M2 phenotype marker CD206+ and the total macrophage phenotype marker F4/80+ accounted for 97%, that was, the type M2 macrophages accounted for 97%. It could be seen that in the repair cell model obtained after the BMDMs were differentiated as induced by the mouse GM-CSF and IL-4 stimulation in this example, the proportion of the type M2 macrophages met the requirement of purity.

In this example, untreated BMDMs (the Control group) and BMDMs obtained by inducing through IL-4 in vitro orientational polarization (the DM+IL-4 group) were subjected to cell identification by flow cytometry, so as to obtain a comparison chart of M2-related inflammatory gene expression as shown in FIG. 73. Referring to FIG. 73, compared with the Control group, the expression levels of M2-related genes in the BMDMs that had been stimulated by IL-4 for 6 hours were all up-regulated. Among them, the expression level of CD206 was up-regulated to 114.000±3.579, the expression level of IL10 was up-regulated to 2.634±0.028, and the expression level of ARG1 was up-regulated to 53.260±8.083.

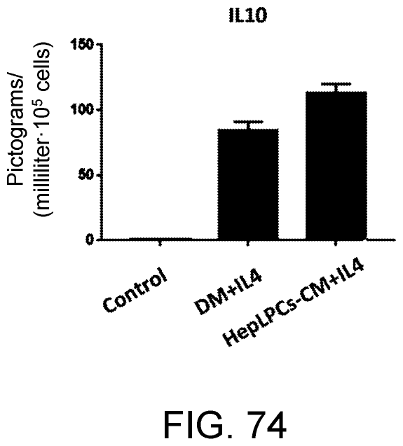

In this example, HepLPCs-CM was co-cultured with the aforementioned BMDMs obtained by inducing through IL-4 in vitro orientational polarization in a BMDM medium for 6 hours to obtain a HepLPCs-CM+IL-4 group, and the HepLPCs-CM+IL-4 group, the Control group and the DM+IL-4 group were subjected to RNA extraction and gene expression analysis to obtain the comparison chart of the secretion of the M2-related inflammatory factor IL10 in each group as shown in FIG. 74. Referring to FIGS. 73 and 74, compared with DM+IL-4, the secretion of the inflammatory factor IL10 in the supernatant was increased as detected after the BMDMs obtained by inducing through IL-4 in vitro orientational polarization were co-cultured with HepLPCs-CM. The secreted IL10 was 108.052±0.472 pg/(mL*10⁵ cells).

It was generally believed in the industry that the type M2 macrophages mainly produce immune regulatory factors such as interleukin-10 (IL-10), which participated in Th2 cell-type immune responses, inhibited inflammation and fibrosis, and played an important role in tissue repair. From FIGS. 73 and 74 and the respective analysis results, it could be seen that HepLPCs-CM could promote IL-4-induced gene expression of repair type M2 macrophages and a small increase of the anti-inflammatory factor IL-10.

As a comprehensive result of Examples 3-2 and 3-3, it could be seen that HepLPCs-CM played a role in inhibiting inflammatory responses and promoting tissue repair by affecting the change of macrophage subpopulations.

### Examples 3-4

In this example, the exosome components (abbreviated as HepLPCs-Ex) in the HepLPCs-CM of Example 3-1 were extracted to investigate its effect on the inflammatory cell model of Example 3-2.

In this example, the exosome component in the HepLPCs-CM of Example 3-1 was extracted by using an ExoQuick-TC exosome extraction kit to obtain HepLPCs-Ex. For specific extraction methods, please refer to the instructions of the kit. The extracted HepLPCs-Ex was entrusted to Shanghai XP Biomed Ltd. for NTA detection. The results showed that the particle diameters of the extracted samples were mostly concentrated between 90-110 nm, with a peak at 96 nm.

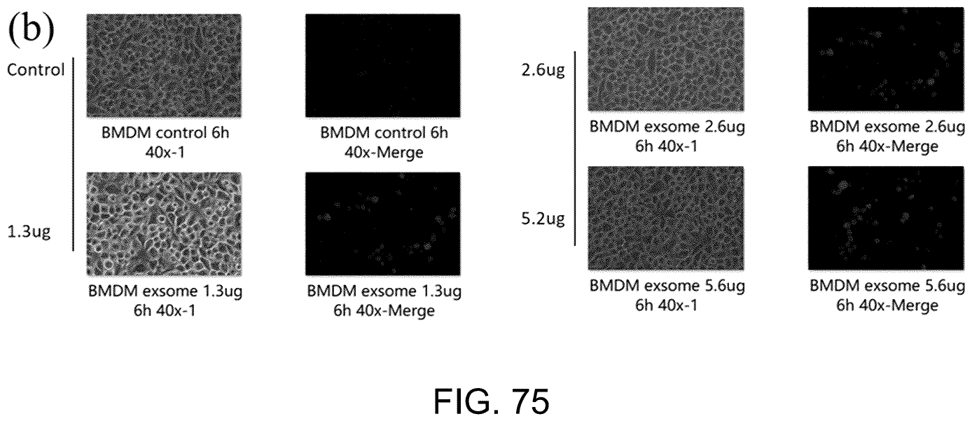

In this example, the HepLPCs-Ex was labelled with DIL, mixed with macrophage BMDMs and then cultured to investigate the phagocytic efficiency of 5×10⁵ macrophage BMDMs on the exosomes at different times and under conditions of different exosome concentrations. It was found that the exosomes phagocytized by the macrophages were more over time, and more macrophages were phagocytized when the concentration of the added exosomes was higher. In this example, the sample after 6 hours of mixed culture was further subjected to nuclear staining with DAPI and fusion analysis by software, so as to obtain a fusion image of exosomes with different concentrations after DAPI nuclear staining as shown in FIG. 75. For the preparation method of the macrophage BMDMs, please refer to Example 2. Referring to FIG. 75, after the concentration of the exosomes was increased from 1.3 ug/uL to 5.2 ug/uL and subjected to mixed culture for 6 hours, the phagocytic effect of the macrophages on the exosomes was all significant, and the phagocytosis efficiency of the macrophages was better when the concentration of the exosomes was higher.

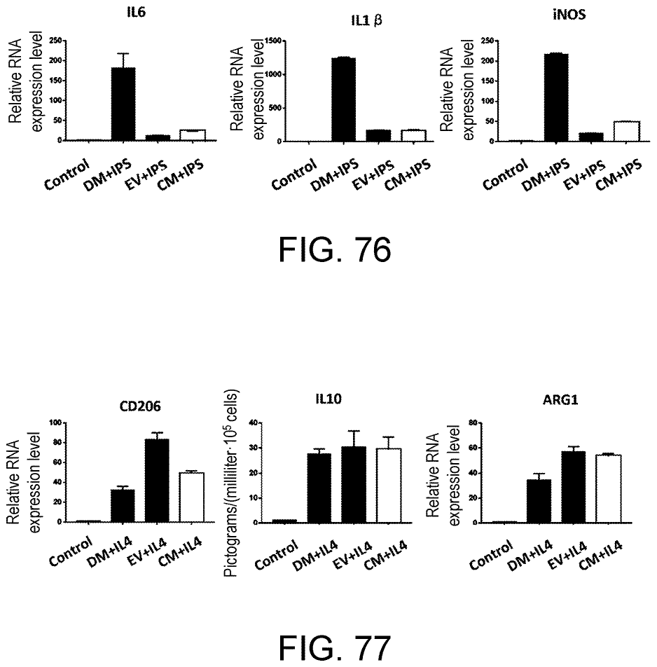

In this example, under the guidance of the condition of the exosomes uptaken by the macrophage as shown in FIG. 75, it was controlled that the number of the BMDMs cells was 5×10⁵, and the concentrations of the exosomes and the HepLPCs-CM were both 1.3 ug/uL. HepLPCs-CM and HepLPCs-Ex were added into the inflammatory cell model, mixed and then cultured for 6 hours to form a CM+LPS group and an EV+LPS group. The expression of M1-related inflammatory genes in the Control group, the DM+LPS group, the CM+LPS group and the EV+LPS group were investigated by qPCR to obtain a comparison chart of levels of M1-related inflammatory genes in each group as shown in FIG. 76.

Referring to FIG. 76, the expression of the M1-related inflammatory genes IL6, IL1β and iNOS in the control group and the DM+LPS group were significant, the expression levels of the aforementioned M1-related inflammatory genes could be reduced significantly by intervention with HepLPCs-CM and HepLPCs-Ex, and the intervention with HepLPCs-Ex allowed the reduction degree of the expression level of the aforementioned M1-related inflammatory genes to be equivalent as the intervention effect of HepLPCs-CM.

In view of the above, the exosome HepLPCs-Ex in the HepLPCs-CM, as an inhibitory component, could inhibit the inflammatory activation of type M1 macrophages.

### Examples 3-5

This example investigated the effect of the exosome component HepLPCs-Ex of Example 3-4 on the repair cell model of Example 3-3.

In this example, under the guidance of the condition of the exosomes uptaken by the macrophage as shown in FIG. 75, it was controlled that the number of the BMDMs cells was 5×10⁵, and the concentrations of the exosomes and the HepLPCs-CM were both 1.3 ug/uL. HepLPCs-CM and HepLPCs-Ex were added into the repair cell model of Example 3, mixed and then cultured for 6 hours to form a CM+IL4 group and an EV+IL4 group. The expression of M2-related inflammatory genes in the Control group, the DM+IL4 group, the CM+IL4 group and the EV+IL4 group were investigated by qPCR to obtain a comparison chart of levels of M2-related inflammatory genes in each group as shown in FIG. 77.

Referring to FIG. 77, compared with the expression levels of M2-related inflammatory genes CD206, IL10 and ARG1 in the control group and the DM+IL4 group, the expression levels of the aforementioned M2-related inflammatory genes could be up-regulated by intervention with either HepLPCs-CM or HepLPCs-Ex, and the intervention with HepLPCs-Ex allowed the up-regulated degree of the expression level of the aforementioned M2-related inflammatory genes to be comparable to the effect of intervention with HepLPCs-CM.

In view of the above, the exosome HepLPCs-Ex in the HepLPCs-CM was used as a promoting component, which could promote the production of repair-type macrophages.

### Examples 3-6

This example provided a modeling method for a mouse NASH model.

In this example, healthy male 5-week-old wild-type (WT) C57BL/6 mice purchased from Shanghai Lingchang Biotechnology Co., Ltd. were used for modeling. The average weight of the mice reached 20 g. There were 7 mice in each group, and the grouping situation was as follows:
model group: the mice were fed with a CDAHFD high-fat feed at a feeding rate of 2.5-3 g/mouse, and the feed was added 2-3 times per week.
Control group: the mice were fed with a standard diet at a feeding rate of 2.5-3 g/mouse.

The mice in each group were quarantined and pre-adapted for 1 week, and then reared in a specific pathogen-free feeding environment (SPF grade) with controlled room temperature of 20-26° C., humidity of 40-70%, and lighting for 12 hours with alternating light and dark. The number of animals in each case was no more than 5.

The mice in the model group were fed for 3 weeks to obtain an early NASH mouse model; and the early NASH mouse model was fed for 6 weeks to obtain a middle-advanced NASH mouse model.

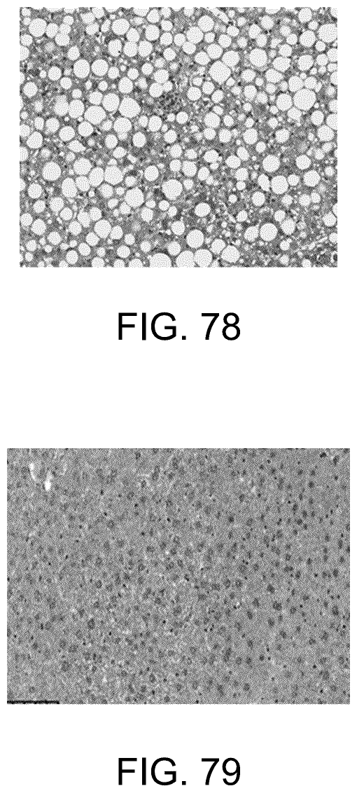

In this example, the liver tissues of the mice in the early NASH mouse model and the control group were taken for paraffin embedding, sample preparation by sectioning and H&E staining, so as to obtain the H&E stained photographs as shown in FIGS. 78 and 79 respectively. Compared with the control group, the hepatocytes in the early NASH mouse model showed ballooning degeneration and were presented with obvious diffuse lipoid degeneration.

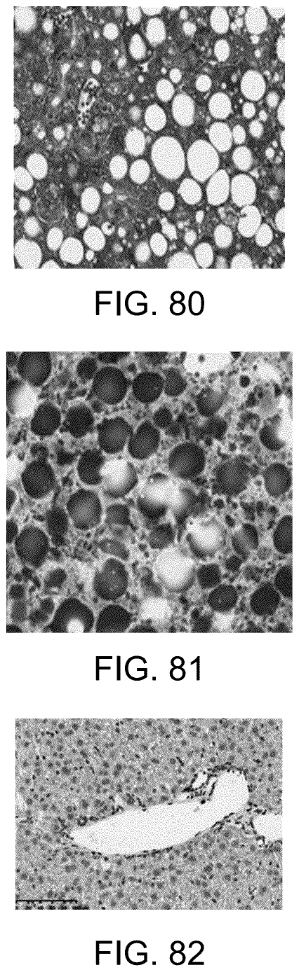

In this example, the liver tissues of the early NASH mouse model was paraffin-embedded and sectioned for sample preparation, and then subjected to Masson staining and oil red O staining respectively to obtain the Masson stained photographs and the oil red O stained photographs as shown in FIGS. 80 and 81 respectively. It could be seen that there was a large amount of lipid deposition in the livers of the mice in the early NASH mouse model.

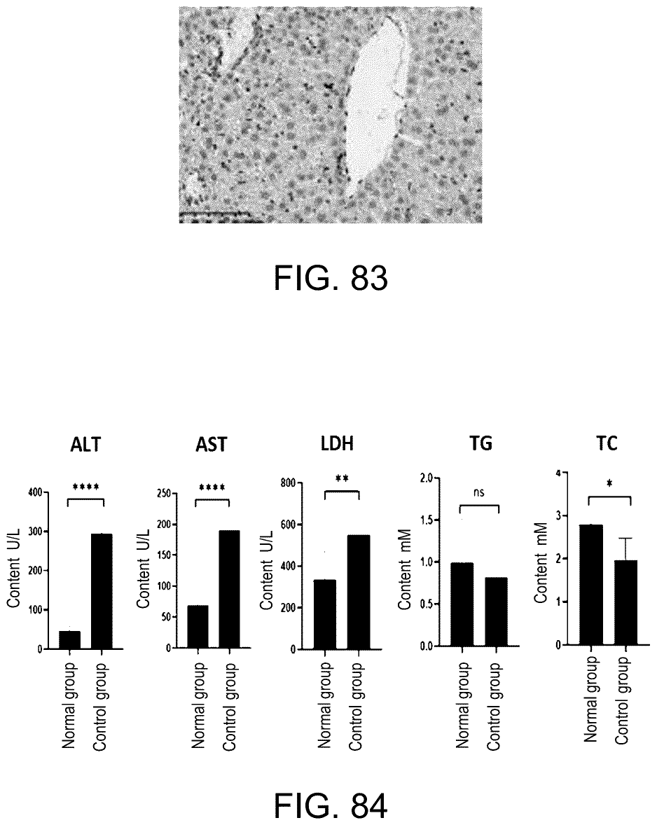

In this example, the liver tissues of the early NASH mouse model was paraffin-embedded and sectioned for sample preparation, and then respectively subjected to immunohistochemical staining of the type M1 macrophage-specific marker CD68 and immunohistochemical staining of the type M2 macrophage-specific marker CD163, so as to obtain the stained photographs as shown in FIGS. 82 and 83. It could be seen that the mouse liver tissue of the early NASH mouse model was accompanied by inflammatory cell infiltration.

In this example, liver function-related indicators AST, ALT, and LDH of blood samples were detected by orbital blood extraction, and the contents of total cholesterol TC and liver triglyceride TG in the ex vivo liver tissues were detected, so as to evaluate the lipid deposition of hepatocytes in the early NASH mouse model and the control group, thereby obtaining a comparison chart of the blood biochemistry and the contents of TC and TG in the early NASH mouse model and the control group as shown in FIG. 84. Referring to FIG. 84, it could be seen that in the early NASH mouse model, the liver blood biochemical indicator AST/ALT was increased by 6-7 times compared with the control group fed with a normal feed, and the LDH was increased by 2 times compared with the normal group, indicating that obvious liver damage occurred in the NASH mice. There was no significant difference in the TG content between the early NASH mouse model and the control group, ΔΔ P<0.05, with no statistical significance; while the TC content was significantly reduced.

It could be seen that the NASH mouse disease model was successfully established after the mice were fed with a special high-fat diet for 3 weeks.

### Examples 3-7

In this example, the early NASH mouse model was intervened with the HepLPCs of Example 3-1 to investigate the effect of HepLPCs on the NASH disease. The early NASH mouse model was grouped as follows, with 7 mice in each group:
model group: orally administrated with physiological saline;
immunosuppressive group: orally administrated with the immunosuppressant FK506 at a dose of 0.2 mg/kg;
group treated with a low dose of cells: orally administrated with 0.2 mg/kg of the immunosuppressant on the day before operation; and during operation each mouse was intraperitoneally injected with 200 microliters of a cell injection which was obtained by resuspending 0.5×106 HepLPCs in physiological saline;
group treated with a high dose of cells: the difference from the group treated with a low dose of cells was that each mouse was injected with a cell injection containing 1×106 HepLPCs; and
sham operation group: each mouse was injected with 200 microliters of physiological saline through the spleen.

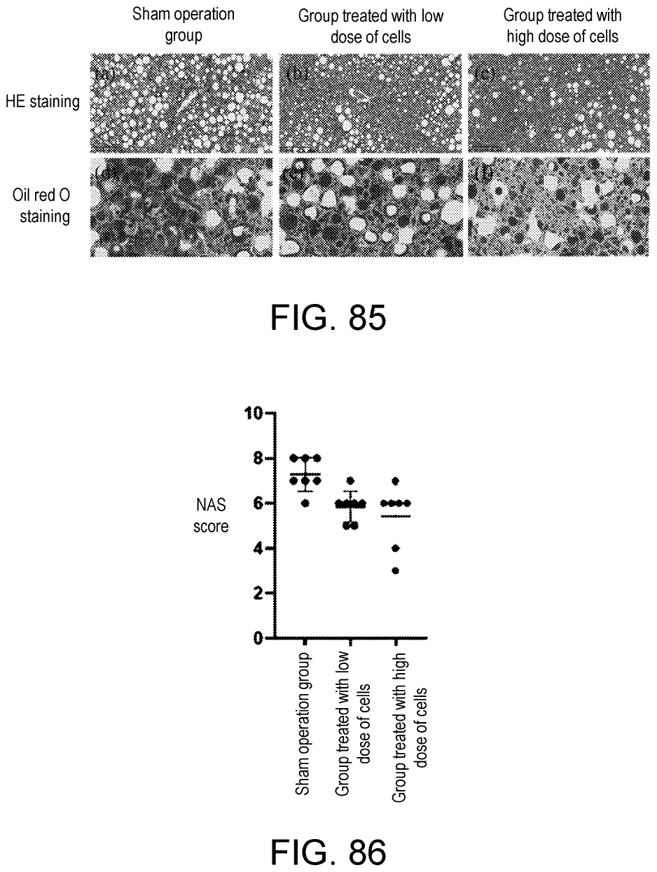

The mice in each of the aforementioned groups were sacrificed on day 10 after completion of the injection or oral administration, and the liver tissues were taken for paraffin embedding, sectioning, and then H&E staining and oil red O staining to observe the liver pathological changes and lipid deposition, so as to obtain the comparison chart of H&E stained photographs and oil red O stained photographs of the livers of the mice in the sham operation group, the group treated with a low dose of cells and the group treated with a high dose of cells as shown in FIG. 85. Referring to FIG. 86, compared with the sham operation group, the group treated with a low dose of cells and the group treated with a high dose of cells showed significantly alleviated lipid deposition in the liver.

In this example, the pathological changes of the liver under a microscope was analyzed by applying NAFLD activity scoring (NAS), so as to obtain a comparison chart of the NAS scores of the sham operation group, the group treated with a low dose of cells and the group treated with a high dose of cells as shown in FIG. 86. Referring to FIG. 86, the NAS score of the sham operation group was between 4-6, which met the pathological evaluation criteria of NASH, while the score of each group treated with cells was lower than 2, indicating that NASH could be ruled out. It showed that the HepLPCs had a positive intervention effect on the early NASH mouse model.

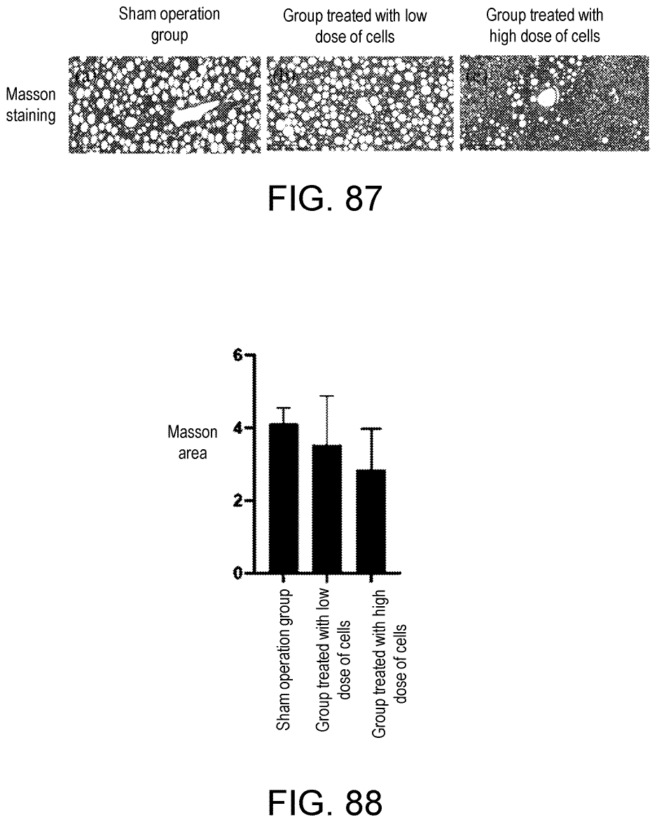

In this example, Masson staining and statistics of liver fibrosis regions were performed on the liver sections of the mice in the sham operation group, the group treated with a low dose of cells and the group treated with a high dose of cells, so as to obtain the Masson stained photograph of the liver sections of the mice in the sham operation group, the group treated with a low dose of cells and the group treated with a high dose of cells as shown in FIG. 87 and a comparison chart of the statistical results of liver fibrosis regions in each group as shown in FIG. 88. It could be seen that, the fibrosis regions in the livers of the mice in the group treated with a low dose of cells and the group treated with a high dose of cells were both significantly reduced compared with that of the sham-operated control group, and especially the reduction in the group treated with a high dose of cells was the most obvious, showing that treatment of the early NASH mouse model with the HepLPCs could have a strong ability to relieve liver fibrosis.

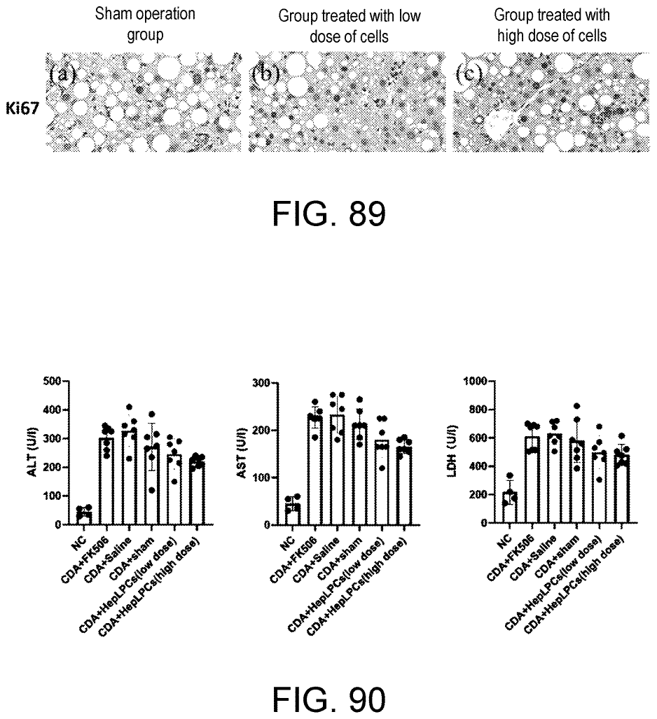

In this example, the liver sections of the mice in the sham operation group, the group treated with a low dose of cells and the group treated with a high dose of cells were stained with Ki67 to obtain the stained photograph as shown in FIG. 89. It could be seen that compared with the sham operation group, the HepLPCs promoted liver regeneration of the mice in the group treated with a low dose of cells and the group treated with a high dose of cells.

In this example, blood was taken from orbits of the mice in the normal control group NC, the immunosuppression group CDA+FK506, the model group CDA+saline administrated orally with physiological saline, the sham operation group CDA+sham, the group treated with a low dose of cells CDA+HepLPCs (low dose) and the group treated with a high dose of cells CDA+HepLPCs (high dose) to investigate the indicators ALT, AST, and LDH of each group through blood biochemical level analysis, so as to obtain the comparison chart of the levels of indicators ALT, AST and LDH in each group as shown in FIG. 90. Compared with the sham operation group, there was a certain amount of decline in the three indicators ALT, AST, and LDH in the two groups treated with cells. A treatment window of the liver function indicator ALT in the group treated with a low dose of cells was decreased by 16%, and a treatment window of the liver function indicator ALT in the group treated with a high dose of cells was decreased by 31.5%; compared with the sham operation group, a treatment window of the liver function indicator AST in the group treated with a low dose of cells was decreased by 15%, and a treatment window of the liver function indicator AST in the group treated with a high dose of cells was decreased by 22%; and compared with the sham operation group, a treatment window of the liver function indicator LDH in the group treated with a low dose of cells was decreased by 13.9%, and a treatment window of the liver function indicator LDH in the group treated with a high dose of cells was decreased by 17%. It could be seen that the HepLPCs could improve the liver function of the early NASH mouse model.

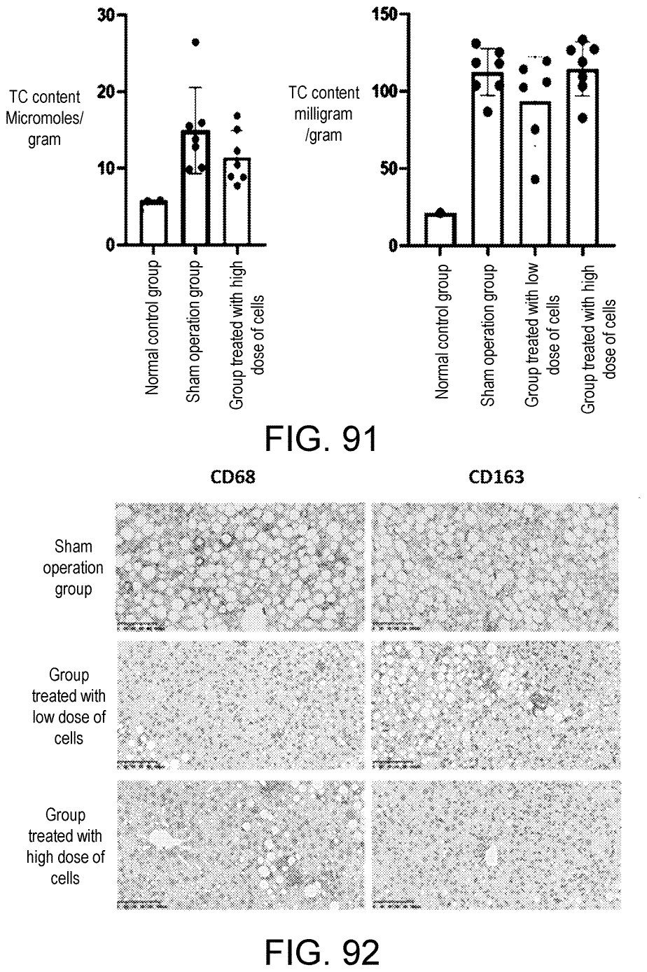

In this example, the contents of TC and TG in the livers of the mice in the normal control group, the sham operation group, the group treated with a low dose of cells and the group treated with a high dose of cells were determined, so as to obtain a comparison chart of TC and TG contents in the livers of each group as shown in FIG. 91. Compared with the sham operation group, the liver cholesterol TC content of the group treated with a high dose of cells was decreased by 23.4%; compared with the sham operation group, the liver triglyceride TG content of the group treated with a low dose of cells was decreased by 17.6%; the liver triglyceride TG content in the group treated with a high dose of cells had no significant difference; from the detection of TC and TG, it could be seen that the contents of TC and TG in the sham operation group were both higher than those in the normal group. Although the abilities of different doses of cells to alleviate the lipid metabolism of a NASH liver were different, but in summary, it was showed that the HepLPCs could significantly improve the lipid metabolism ability of the NASH liver.

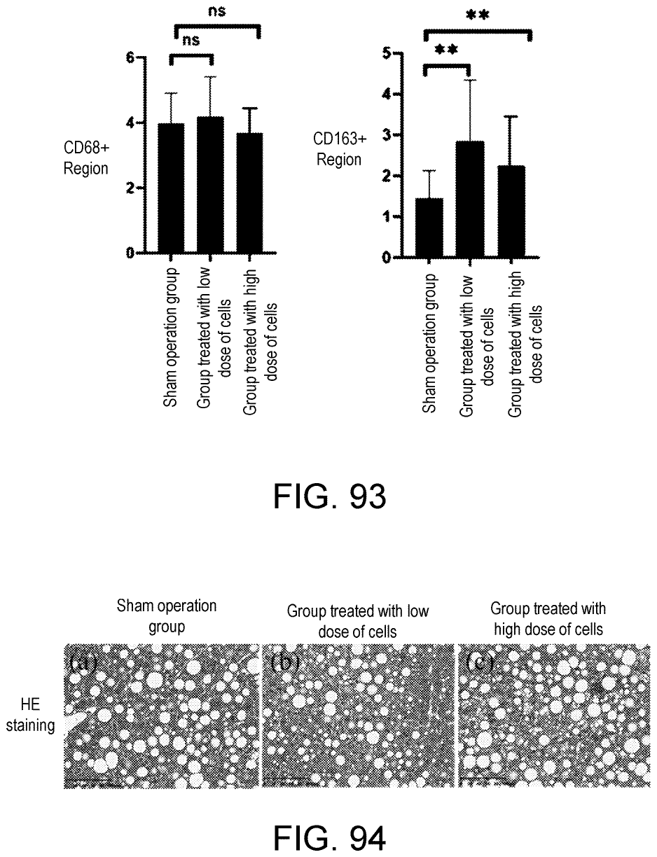

In this example, the liver tissues of the mice in the sham operation group, the group treated with a low dose of cells and the group treated with a high dose of cells were subjected to anti-CD163 and anti-CD68 immunohistochemical staining, and the CD163⁺ macrophages and the CD68⁺ macrophages in the livers of different groups were counted to obtain a comparison chart of photographs obtained after anti-CD163 and anti-CD68 immunohistochemical staining in each group as shown in FIG. 92 and a comparison chart of the number of CD163⁺ macrophages and CD68⁺ macrophages in the livers of each group as shown in FIG. 93. Compared with the mice in the sham operation group, the number of CD163⁺ macrophages in the livers was significantly increased and the number of CD68⁺ macrophages was slightly decreased in the mice of each of the two groups treated with cells. These results suggested that the HepLPCs inhibited macrophage-related inflammatory responses and promoted the production of repair-type macrophages.

### Examples 3-8

In this example, the middle-advanced NASH mouse model was intervened with the HepLPCs of Example 3-1 to investigate the effect of HepLPCs on the NASH disease. For the grouping treatment of the middle-advanced NASH mouse model, please refer to Example 3, except that the cell injection infused into the spleen of each mouse in the group treated with a low dose of cells contained 1×10⁶ cells, and the cell injection infused into the spleen of each mouse in the group treated with a high dose of cells contained 2×10⁶ cells.

In this example, the liver sections of the mice in the sham operation group, the group treated with a low dose of cells and the group treated with a high dose of cells of the middle-advanced NASH mouse model were subjected to H&E staining, so as to obtain the stained photograph as shown in FIG. 94. It could be seen that compared with the sham operation group, the livers of the mice in the group treated with a low dose of cells and the group treated with a high dose of cells showed improved lipid deposition.

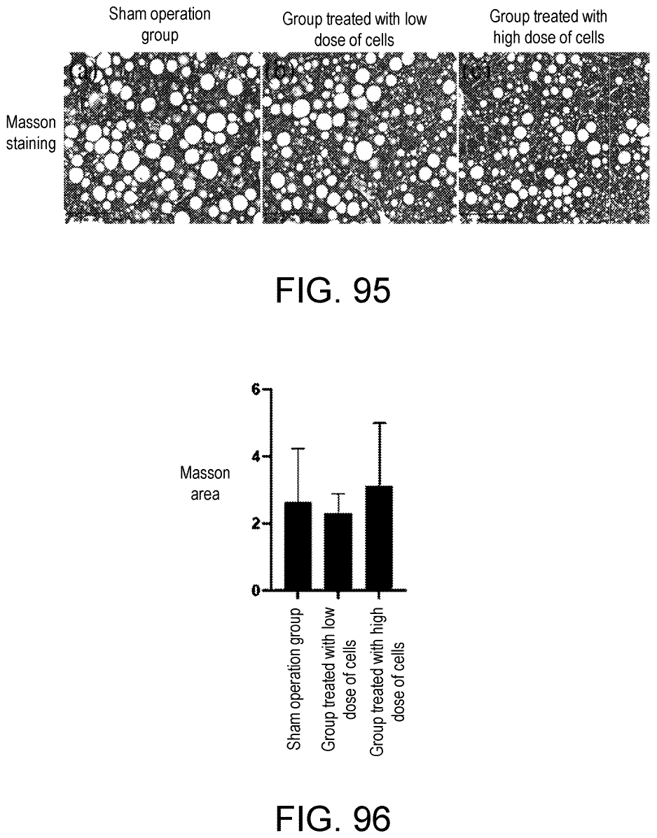

In this example, In this example, the liver sections of the mice in the sham operation group, the group treated with a low dose of cells and the group treated with a high dose of cells of the middle-advanced NASH mouse model were subjected to Masson staining, and the fibrosis regions were counted, so as to obtain a comparison chart of Masson stained photographs of each group as shown in FIG. 95 and a comparison chart of statistical results of liver fibrosis regions in each group as shown in FIG. 96. The fibrosis regions in the livers of the mice in the group treated with a low dose of cells and the group treated with a high dose of cells were significantly reduced, and especially the reduction was the most obvious in the group treated with a low dose of cells. It could be seen that the HepLPCs had a stronger ability of alleviating the liver fibrosis of the middle-advanced NASH mouse model.

### Examples 3-9

This example provided a modeling method of an inflammatory cell model.

Currently, a large number of experimental and clinical data supported the central role of macrophages in the occurrence and development of nonalcoholic fatty liver disease (NAFLD) and nonalcoholic steatohepatitis (NASH). Under the induction with different factors, two classic cell subpopulations of the macrophages could occur, namely the type M1 macrophages and the type M2 macrophages. The functions of the two were completely different. It was generally believed in the industry that the type M1 macrophages promoted inflammatory responses by producing a large number of pro-inflammatory cytokines such as interleukin-1l (IL-10), TNF-α, interleukin-6 (IL-6), as well as nitric oxide (NO) and reactive oxygen species (ROSs) to mediate the inflammatory response of a body.

Therefore, it was necessary to establish an inflammatory cell model, so as to investigate the effect of the HepLPCs on the type M1 macrophages through the inflammatory cell model.

Specifically, 6-8-week-old adult C57 mice were sacrificed by spinal dislocation, and the bone marrow mass in a femoral myelocavity was taken; the bone marrow mass was filtered through a 70 μm cell sieve and centrifuged at 500 g for 5 minutes, and then the supernatant was discarded; the precipitate was resuspended with an erythrocyte lysis buffer (available from Shanghai beyotime Biotechnology Co., Ltd.) and centrifuged again at a centrifugal force of 500 g for 5 minutes, and the supernatant was discarded to obtain a secondary precipitate; and the secondary precipitate was repeatedly resuspended in a BMDM medium and centrifuged to fully wash off the residual erythrocyte lysis buffer and cell debris in the precipitate. Finally, the purified cells were resuspended in a BMDM medium, and then inoculated into a 12-well plate according to (8-10)×10⁵/well, wherein each well was added with mouse GM-CSF at a controlled concentration of 40 ng/mL. 6-8 hours after completion of inoculation, the cells were transferred into a new culture dish for culture, and the medium was replaced with a BMDM medium supplemented with 40 ng/mL of mouse GM-CSF once every 3 days until day 7, so as to achieve the differentiation and maturation of macrophages. The BMDM medium consisted of 500 mL of a 1640 culture medium (available from Shanghai Basalmedia Technologies Co., Ltd.), 5% of a penicillin-streptomycin double antibody and 10% of FBS.

Specifically, after the macrophages were differentiated and matured, the culture medium was replaced with a BMDM medium containing 100 ng/mL of LPS, and culture was conducted for 6 hours to complete the induction by orientational polarization in vitro.

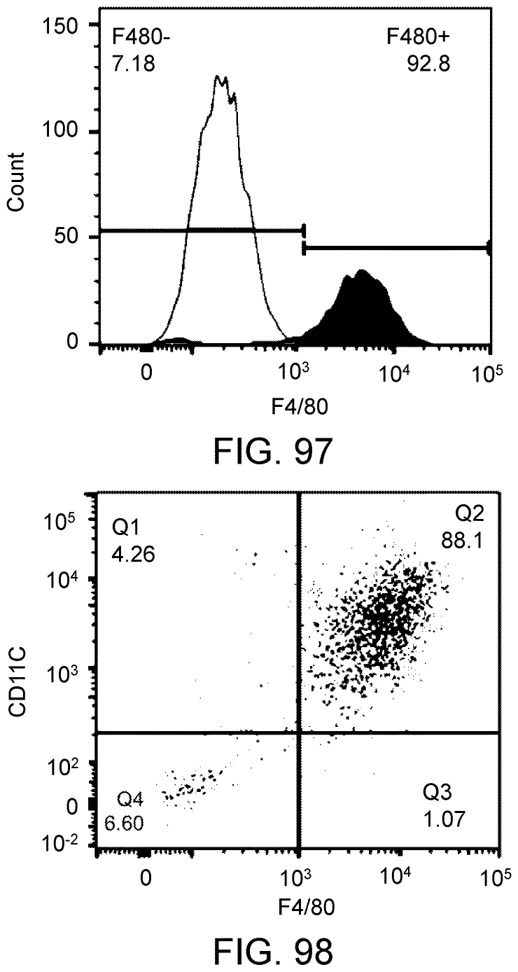

In this example, the BMDMs obtained by inducing with the mouse GM-CSF and the BMDMs obtained by inducing with LPS in vitro orientational polarization were subjected to cell identification by flow cytometry, so as to obtain the analysis result of the proportion of total macrophages as shown in FIG. 97 and the analysis result of the proportion of the type M1 macrophages as shown in FIG. 98. The expression of the total macrophage phenotype marker F4/80+ in the BMDMs obtained by inducing with the mouse GM-CSF accounted for 92.8%, that was, the total macrophage accounted for 92.8%, which was line with the experimental cell purity of BMDMs; and in the BMDMs obtained after LPS stimulation for 6 hours, the expression of M1 phenotype markers CD11c+ and F4/80+ accounted for 88.1%, that was, the type M1 macrophages accounted for 88.1%. It could be seen that in the inflammatory cell model obtained after the BMDMs were differentiated as induced by the mouse GM-CSF and LPS stimulation in this example, the proportion of the type M1 macrophages met the requirement of purity.

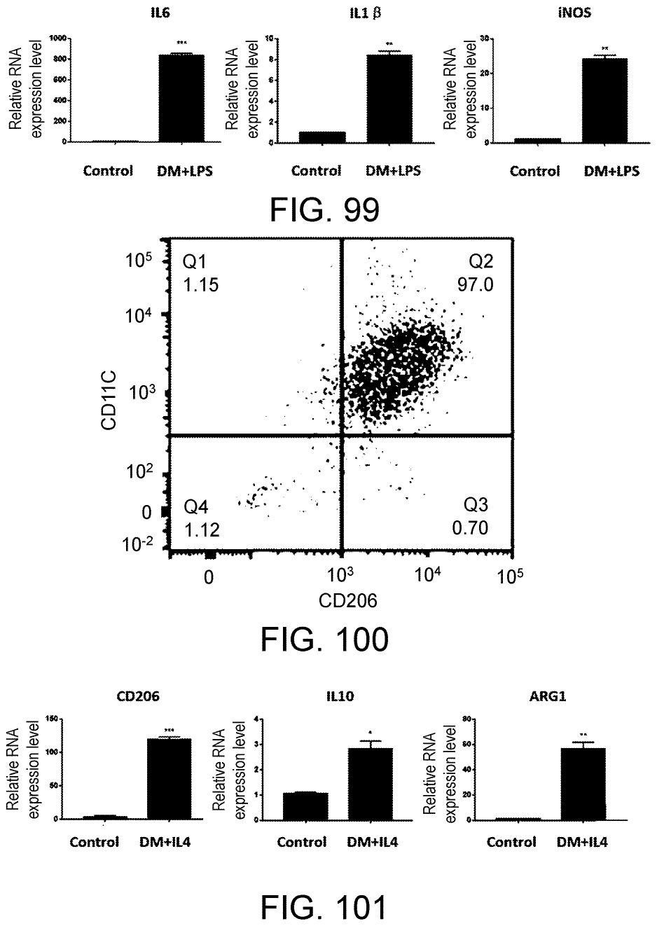

In this example, untreated BMDMs (the Control group) and BMDMs obtained by inducing through LPS in vitro orientational polarization (the DM+LPS group) were subjected to cell identification by flow cytometry, so as to obtain a comparison chart of M1-related inflammatory gene expression as shown in FIG. 99. Referring to FIG. 99, compared with the control group, the expression levels of M1-related genes in the BMDMs that had been stimulated by LPS for 6 hours were all up-regulated. Among them, the expression level of IL6 was up-regulated to 823.200±174.500, the expression level of IL1β was up-regulated to 8.389±0.029, and the expression level of iNOS was up-regulated to 24.650±1.196.

### Examples 3-10

This example provided a modeling method of a repair macrophage model.

It was generally believed in the industry that the type M2 macrophages mainly produce immune regulatory factors such as interleukin-10 (IL-10), which participated in Th2 cell-type immune responses, inhibited inflammation and fibrosis, and played an important role in tissue repair.

Therefore, it is necessary to establish a repair macrophage model, so as to further investigate the effect of the HepLPCs on the type M2 macrophages through the repair macrophage model.

In this example, the repair macrophage model was established by stimulating macrophages with IL-4. The specific process included that: for the method of acquiring mouse primary bone marrow-derived macrophages (BMDMs) and the method of conducting differentiation of the BMDMs with the mouse GM-CSF for 7 days, please refer to Example 3-2. The mature primary macrophages were induced by in vitro orientational polarization with IL-4 for 6 hours, and then the macrophages were digested to obtain the repair macrophage model. Among them, the concentration of IL-4 was controlled to be 40 ng/mL during the process of stimulation with IL-4. For other specific experimental steps, please refer to Example 5.

In this example, the BMDMs induced by IL-4 in vitro orientational polarization were subjected to cell identification by flow cytometry, so as to obtain the analysis result of the proportion of type M2 macrophages as shown in FIG. 100. It could be seen that in the BMDMs obtained after IL-4 stimulation for 6 hours, the expression of the M2 phenotype marker CD206+ and the total macrophage phenotype marker F4/80+ accounted for 97%, that was, the type M2 macrophages accounted for 97%. It could be seen that in the repair macrophage model obtained after the BMDMs were differentiated as induced by the mouse GM-CSF and IL-4 stimulation in this example, the proportion of the type M2 macrophages met the requirement of purity.

In this example, untreated BMDMs (the Control group) and BMDMs obtained by inducing through IL-4 in vitro orientational polarization (the DM+IL-4 group) were subjected to cell identification by flow cytometry, so as to obtain a comparison chart of M2-related inflammatory gene expression as shown in FIG. 101. Compared with the Control group, the expression levels of M2-related genes in the BMDMs after 6 hours of IL-4 stimulation were all up-regulated. Among them, the expression level of CD206 was up-regulated to 114.000±3.579, the expression level of IL10 was up-regulated to 2.634±0.028, and the expression level of ARG1 was up-regulated to 53.260±8.083.

### Examples 3-11

In this example, the HepLPCs of Example 3-1 were cultured in vitro, and an exosome component (abbreviated as HepLPCs-Ex) was extracted from the resultant in vitro culture supernatant to investigate its effect on the inflammatory cell model of Example 3-5.

A process of culturing the HepLPCs of Example 3-1 in vitro and acquiring the in vitro culture supernatant (HepLPCs-CM) included: after the subculture of Example 1 was completed, the TEM medium was replaced with a high-glucose serum-free DMEM medium, and in vitro culture was continued for 24 hours. After the in vitro culture was completed, the cell supernatant was collected and centrifuged at a centrifugal force of 300 g for 10 minutes to remove cell debris, so as to obtain the in vitro culture supernatant.

In this example, the exosome component in the HepLPCs-CM was extracted by using an ExoQuick-TC exosome extraction kit to obtain HepLPCs-Ex. For specific extraction methods, please refer to the instructions of the kit. The extracted HepLPCs-Ex was entrusted to Shanghai XP Biomed Ltd. for NTA detection. The results showed that the particle diameters of the extracted samples were mostly concentrated between 90-110 nm, with a peak at 96 nm.

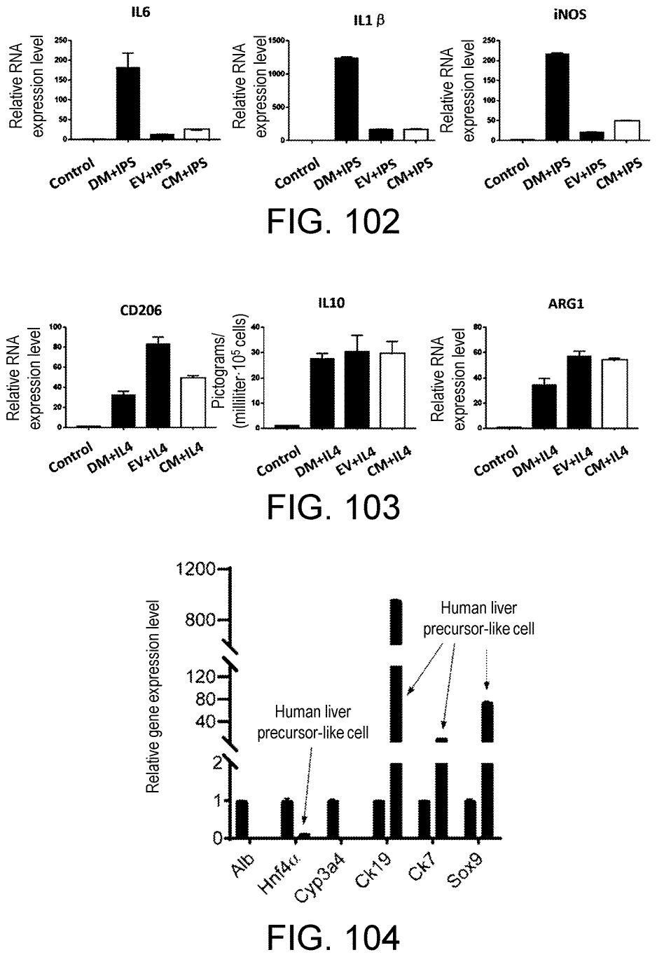

In this example, it was controlled that the number of the BMDMs cells was 5×10⁵, and the concentrations of the exosomes and the HepLPCs-CM were both 1.3 ug/uL. HepLPCs-CM and HepLPCs-Ex were added into the inflammatory cell model, mixed and then cultured for 6 hours to form a CM+LPS group and an EV+LPS group. The expression of M1-related inflammatory genes in the Control group, the DM+LPS group, the CM+LPS group and the EV+LPS group were investigated by qPCR to obtain a comparison chart of levels of M1-related inflammatory genes in each group as shown in FIG. 102.

Referring to FIG. 102, the expression of the M1-related inflammatory genes IL6, IL1β and iNOS in the control group and the DM+LPS group were significant, the expression levels of the aforementioned M1-related inflammatory genes could be reduced significantly by intervention with HepLPCs-CM and HepLPCs-Ex, and the intervention with HepLPCs-Ex allowed the reduction degree of the expression level of the aforementioned M1-related inflammatory genes to be equivalent as the intervention effect of HepLPCs-CM.

In view of the above, the exosome HepLPCs-Ex released by HepLPCs as induced by the serum-free basal medium could inhibit the inflammatory activation of the type M1 macrophages.

### Examples 3-12

This example investigated the effect of the exosome component HepLPCs-Ex of Example 3-10 on the aforementioned repair macrophage model.

In this example, it was controlled that the number of the BMDMs cells was 5×10⁵, and the concentrations of the exosomes and the HepLPCs-CM were both 1.3 ug/uL. HepLPCs-CM and HepLPCs-Ex were added into the repair macrophage model of Example 6, mixed and then cultured for 6 hours to form a CM+IL4 group and an EV+IL4 group. The expression of M2-related inflammatory genes in the Control group, the DM+IL4 group, the CM+IL4 group and the EV+IL4 group were investigated by qPCR to obtain a comparison chart of levels of M2-related inflammatory genes in each group as shown in FIG. 103.

Referring to FIG. 103, compared with the expression levels of M2-related inflammatory genes CD206, IL10 and ARG1 in the control group and the DM+IL4 group, the expression levels of the aforementioned M2-related inflammatory genes could be up-regulated by intervention with either HepLPCs-CM or HepLPCs-Ex, and the intervention with HepLPCs-Ex allowed the up-regulated degree of the expression level of the aforementioned M2-related inflammatory genes to be comparable to the effect of intervention with HepLPCs-CM.

In view of the above, the exosome HepLPCs-Ex released by the HepLPCs as induced by the serum-free basal medium could promote the production of the repair macrophages, i.e., the type M2 macrophages.

The following examples prepared immune cell proliferation inhibitors and proliferation inhibitory cell preparations respectively as liver disease regulatory formulations, and the use of such a liver disease regulatory formulation was investigated. The details were as follows:

### Examples 4-1

In this example, human primary hepatocytes derived from the donor 1 available from Invitrogen were cultured in vitro to serve as liver precursor-like cells HepLPC, and the conditional culture supernatant (HepLPC-CM) was acquired. Then the HepLPC-CM was co-cultured with ConA-stimulated mouse spleen cells to investigate the inhibitory effect of the HepLPC-CM on the proliferation of spleen cells.

A method for culturing the HepLPC in vitro included: the human primary hepatocytes were subjected to Percoll density gradient centrifugation combined with flow sorting to exclude CD24-positive and EpCAM-positive precursor cells, then firstly inoculated into a 6-well plate coated with Matrigel (Corning) at a density of 2×10⁴ cells/cm² and cultured in a WE medium (Invitrogen) containing 10% serum until adherence, and then transferred into a TEM medium at an inoculation density of 2×10⁴ cells/cm² for 10 days of culture, and the TEM medium was replaced once every other day.

The composition of the TEM medium and the sources of individual constituent components of this example were as follows: a DMEM/F12 basal medium (Invitrogen), as well as based on the content in the DMEM/F12 basal medium: an N2 additive with a volume content of 1% and a B27 additive with a volume content of 1% (Invitrogen), 1 mmol/L of sodium pyruvate (Invitrogen), 10 μg/mL of ascorbic acid (Sigma-Aldrich), 20 ng/mL of a hepatocyte growth factor HGF (Peprotech), 20 ng/mL of an epidermal cell growth factor EGF (Peprotech), 10 μmol/L of a ROCK kinase inhibitor Y27632 (TargetMol), 3 μmol/L of a Wnt signaling pathway agonist CHIR99021 (TargetMol), 1 μmol/L of a TGF-β signal inhibitor A83-01 (TargetMol), 1 μmol/L of sphingosine monophosphate S1P (Santa Cruz) and 5 μmol/L of indoleacetic acid LPA (Santa Cruz).

A method for preparing HepLPC-CM included: the precursor-like cells obtained by culturing in the TEM culture for 10 days were digested with 0.25% Trypsin-EDTA (available from Gibco, USA), inoculated into a new culture medium according to a ratio of 1:3, subjected to expansion culture to the third generation until the cell confluency reached 80%, and then the TEM medium was replaced with a serum-free DMEM medium and in vitro culture was conducted for 24 hours; and after the in vitro culture was completed, the cell supernatant was collected and centrifuged at a centrifugal force of 300 g for 10 minutes to remove cell debris, so as to obtain the conditional culture supernatant HepLPC-CM.

A method for preparing a suspension of mouse spleen cells added with a stimulator was: 1-week-old C57BL/6 mice were sacrificed by neck dislocation, then the spleen was taken, ground with a 200-mesh sieve, and then rinsed with a lymphatic separation fluid, and the separation solution containing lymphocytes was collected, resuspended by using a RPMI-1640 complete medium containing 10% fetal bovine serum and centrifuged under a centrifugal force of 800 g at low temperature for 30 minutes; and after completion of centrifugation, the lymphocyte layer was pipetted, then resuspended with a RPMI-1640 complete medium containing 10% fetal bovine serum, and then centrifuged under a centrifugal force of 250 g for 10 minutes at low temperature. The obtained lymphocyte layer was resuspended with the RPMI-1640 complete medium containing 10% fetal bovine serum again, and added with a concanavalin A(ConA) suspension resuspended with the RPMI-1640 complete medium containing 10% fetal bovine serum as a stimulator.

The co-culturing step included: the aforementioned mouse spleen cell suspension added with the stimulator was inoculated into a 6-well plate at a density of 2×10⁴ cells/cm²; HepLPC-CM derived from the donor 1 was resuspended by using the HepLPC-CM and the RPMI-1640 complete medium containing 10% fetal bovine serum to obtain HepLPC-CM suspensions with HepLPC-CM concentrations of 100%, 50%, 25%, 12.5% and 6.25%, respectively. The aforementioned HepLPC-CM suspensions were respectively added into the 6-well plate inoculated with the mouse spleen cells, with the final concentration of ConA being controlled at 2.5 micrograms/milliliter to form the following co-culture groups: a 100%-CM group, a 50%-CM group, a 25%-CM group, a 12.5%-CM group and a 6.25%-CM group. Moreover, the co-culture group without addition of HepLPC-CM was used as the positive control group.

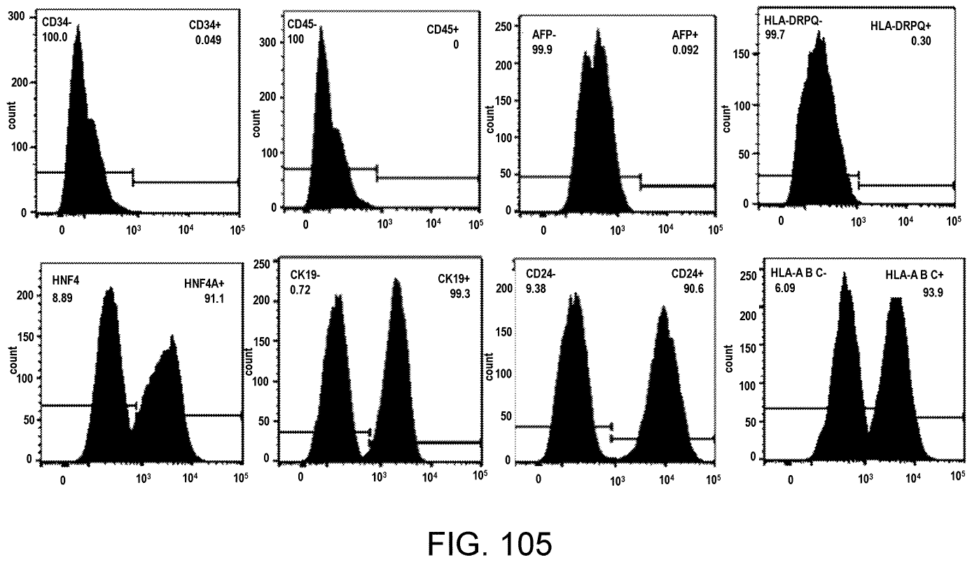

In this example, qPCR and flow cytometry were used for analyzing the gene expression of the human primary hepatocytes derived from the donor 1 and the HepLPC obtained by culturing in TEM, and the results were as shown in FIGS. 104 and 105. Under the action of the TEM medium, in the human primary hepatocytes, the expression of liver progenitor cell genes Ck7, Ck19 and Sox9 were increased significantly, while the expression of liver parenchymal cell markers such as Alb, Cyp3a4 and Hnf4α were decreased significantly. In the Human-HepLPCs, the liver cell marker HNF4α and liver stem cell/hepatic progenitor cell markers CD24 and CK19 were significantly expressed, and the expression levels of a hematopoietic stem cell antigen CD34, a leukocyte common antigen CD45 and a liver fetal cell marker AFP were all less than 2%. The Human-HepLPCs did not express the MHC class II antigens HLA-DP, HLA-DQ and HLA-DR, showing low immunogenicity.

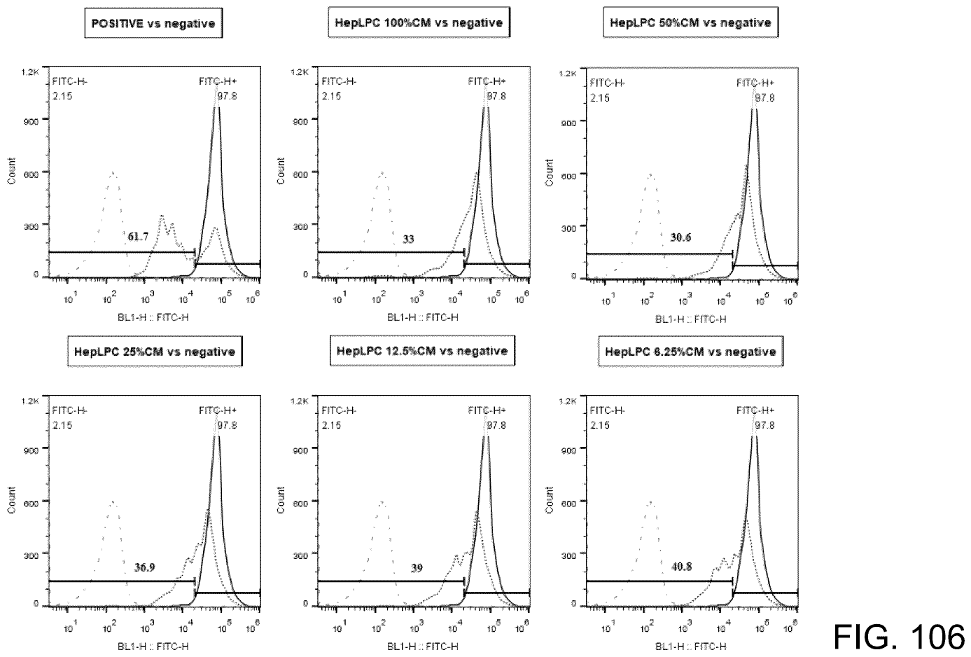

In this example, after 72 hours of co-culture, the positive control group and each co-culture group were treated with a CFSE cell proliferation detection kit and then loaded onto a flow cytometer for detection to investigate the inhibition of HepLPC-CM on proliferation of spleen cells in each co-culture group, and the results were as shown in FIG. 106. It could be seen from the proportions of proliferated spleen cells in the positive control group and each co-culture group as shown in FIG. 106 that, in the 100%-CM group, the 50%-CM group, the 25%-CM group, the 12.5%-CM group and the 6.25%-CM group, the inhibition rates of the HepLPC-CM on proliferation of spleen cells were 47%, 50%, 40%, 37% and 34%, respectively. It could be seen that the HepLPC-CM could significantly inhibit the proliferation of mouse spleen cells in a dose-dependent manner.

### Examples 4-2

In this example, human primary hepatocytes derived from different donor sources available from Invitrogen were cultured in vitro into hepatic precursor-like cells HepLPC, and the conditional culture supernatants from different donor sources were acquired, and then the conditional culture supernatants from different donor sources were co-cultured with ConA-stimulated mouse spleen cells to investigate the inhibitory effect of the HepLPC-CM on the proliferation of spleen cells.

For the in vitro culture method of human primary hepatocytes, the preparation method of the conditional culture supernatant, and the preparation method of mouse spleen cell suspension added with the stimulator, please refer to Example 4-1. The co-culture step was different from that of Example 1 in that: the HepLPC from each donor source was directly co-cultured with mouse spleen cells added with the stimulator, rather than being resuspended with the RPMI-1640 complete medium containing 10% fetal bovine serum.

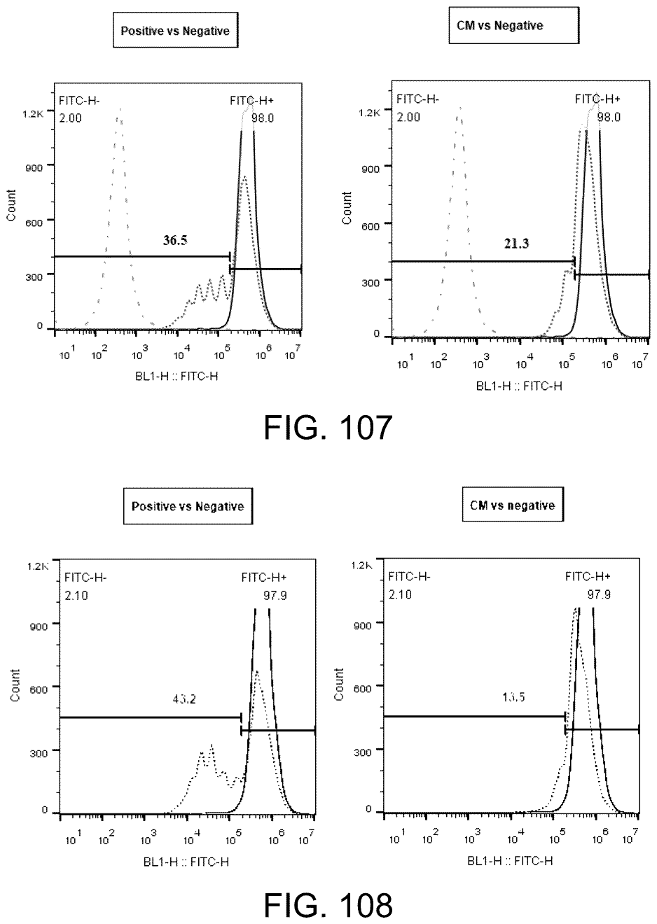

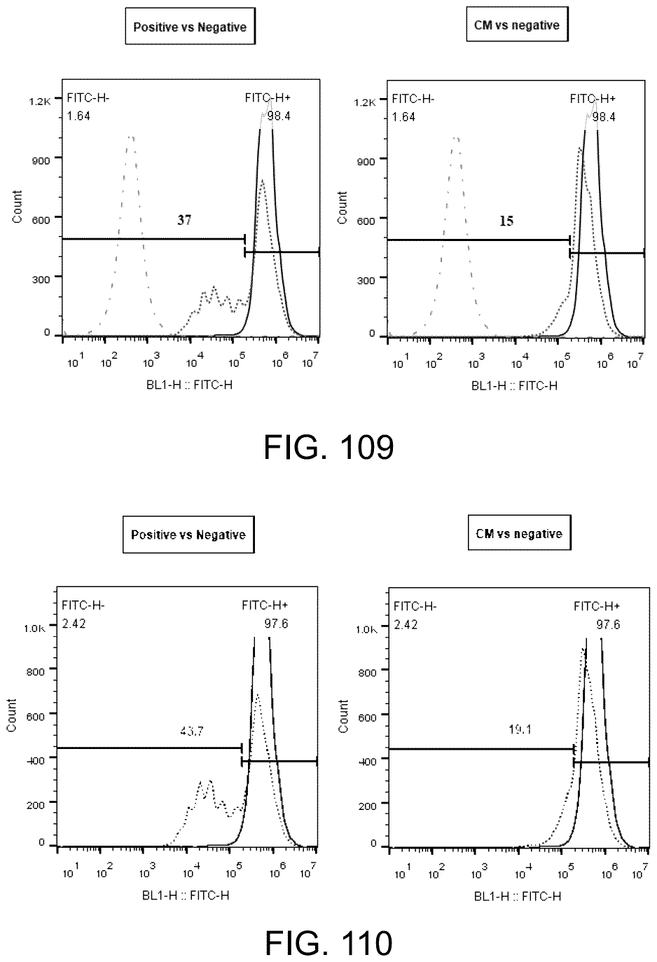

In this example, after 72 hours of co-culture, the positive control group and co-culture groups containing HepLPC-CMs derived from different donor sources were treated with the CFSE cell proliferation detection kit, and then loaded onto a flow cytometer for detection to investigate the inhibition of the co-culture group containing the HepLPC-CM derived from each donor source on the proliferation of the spleen cells and the proliferation of the spleen cells in the corresponding positive control group, and the results were as shown in FIGS. 107 to 110. The inhibition rates of the HepLPC-CMs derived from the donor 1, the donor 2, the donor 3 and the donor 4 on proliferation of spleen cells were 42%, 69%, 59% and 56%, respectively. It could be seen that the inhibitory effects of the HepLPC-CMs derived from different donor sources on proliferation of the spleen cells were different, which is conducive to the subsequent research on the heterogeneity of immune-resistant drugs.

### Examples 4-3

In this example, human primary hepatocytes derived from the donor 1 available from Invitrogen were cultured in vitro to serve as liver precursor-like cells HepLPC, and then co-cultured with ConA-stimulated mouse spleen cells to investigate the inhibitory effect of the HepLPC-CM on the proliferation of spleen cells.

For the in vitro culture method of HepLPC, the composition of the TEM medium and its constituent components, and the preparation method of the mouse spleen cell suspension added with the stimulator, please refer to the foregoing examples.

The co-culture step included: after the aforementioned TEM culture was completed and the cell confluence reached 90%, the obtained cells were successively subcultured in a TEM medium at an inoculation density of 1×10⁴ cells/square centimeter according to a proportion of 1:3 to the third generation; the aforementioned HepLPC subcultured to the third generation and having a cell confluence of 80% was digested with a 0.05% trypsin/EDTA solution, then digestion was stopped by using the RPMI-1640 complete medium containing 10% fetal bovine serum and the cells were resuspended into a single-cell suspension; the single-cell suspension was repeatedly washed with PBS to ensure the removal of the digestion solution; the cells were resuspended by using the RPMI-1640 complete medium containing 10% fetal bovine serum and the cell concentration was adjusted; the aforementioned mouse spleen cell suspension added with the stimulator was inoculated into a 6-well plate, and by controlling the addition amount of the mouse spleen cell suspension added with the stimulator, the number ratios of the HepLPC to mouse spleen cells were adjusted to 1:1, 1:2, 1:5, 1:10 and 1:20, respectively, so as to obtain several co-culture groups. Moreover, the co-culture group without addition of the HepLPC was used as the positive control group, and the co-culture group added with an immunosuppressive drug tacrolimus FK506 at a final concentration of 10 nM was used as the FK506 control group.

In this example, qPCR and flow cytometry were used for analyzing the gene expression of the human primary hepatocytes derived from the donor 1 and the HepLPC obtained by culturing in TEM, with reference to the foregoing examples.

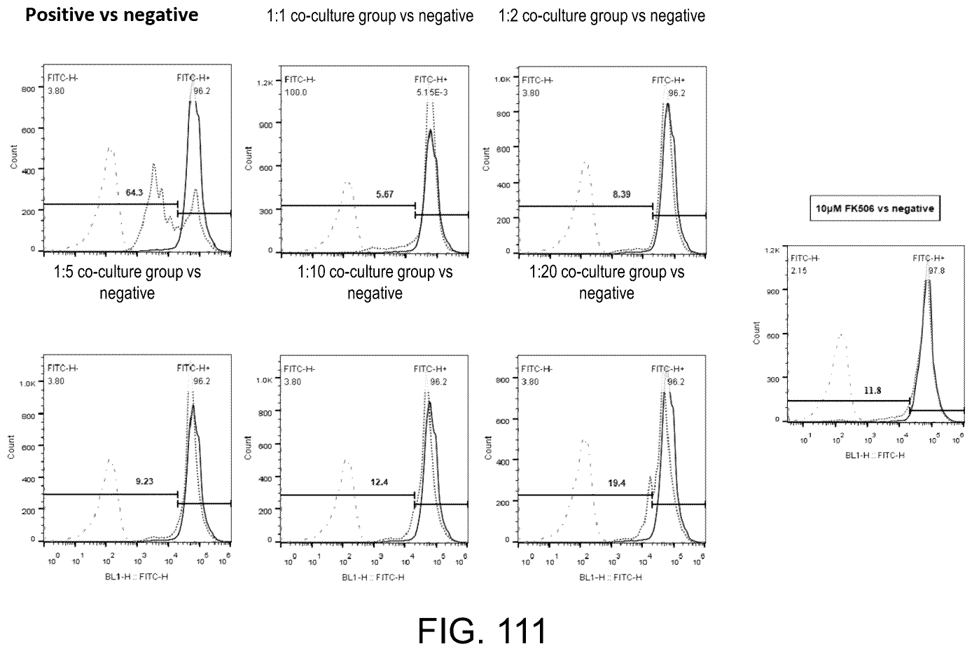

In this example, after 72 hours of co-culture, the positive control group, a FK506 control group and each co-culture group were treated with a CFSE cell proliferation detection kit and then loaded onto a flow cytometer for detection to investigate the inhibition of HepLPC and FK506 on proliferation of spleen cells in each co-culture group, and the results were as shown in FIG. 111. It could be seen from the proportions of proliferated spleen cells in the positive control group, the FK506 control group and each co-culture group as shown in FIG. 111 that, under the conditions of the number ratios of the HepLPC to the mouse spleen cells of respectively 1:1, 1:2, 1:5, 1:10 and 1:20, the inhibition rates of the HepLPC on proliferation of the spleen cells were 90%, 87%, 85%, 80% and 50%, respectively. It could be seen that the inhibitory effect of the HepLPC proliferation of spleen cells was dose-dependent. It was particularly worth noting that the inhibition rate of the FK506 control group on the proliferation of spleen cells was 82%. When the number ratio of the HepLPC to the mouse spleen cells was in a wide range of 1:1-1:5, the proliferation inhibitory effect on proliferation of the spleen cells exhibited by HepLPC was equivalent to that of FK506 or even stronger than that of FK506.

### Example 4-4

In this example, human primary hepatocytes derived from different donor sources available from Invitrogen were cultured in vitro to serve as liver precursor-like cells HepLPC, and HepLPCs derived from different donor sources were co-cultured with ConA-stimulated mouse spleen cells to investigate the inhibitory effect of HepLPC on the proliferation of spleen cells.

For the in vitro culture method of human primary hepatocytes, the preparation method of the conditional culture supernatant, the preparation method of mouse spleen cell suspension added with the stimulator and the co-culture method, please refer to Example 4-1. In the co-culture step, the number ratio of the HepLPC derived from each donor to the mouse spleen cells was controlled to be 1:5, respectively.

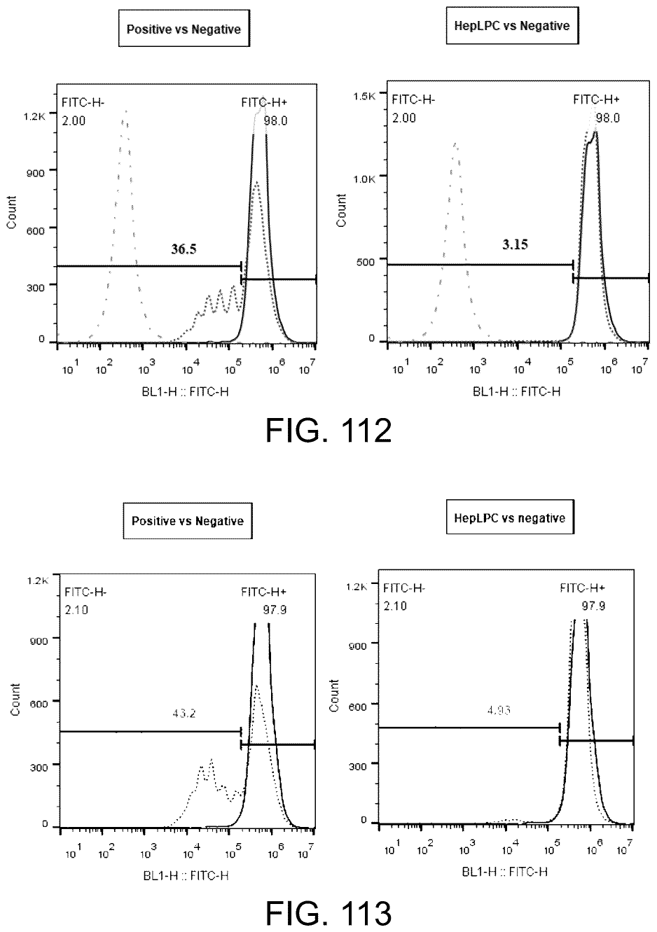

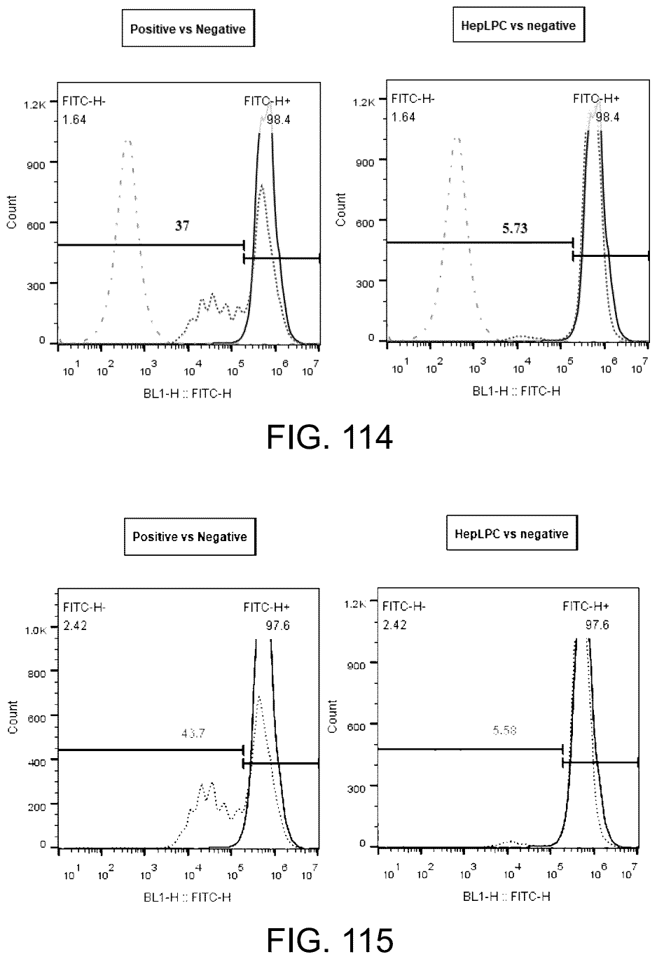

In this example, after 72 hours of co-culture, the positive control group and co-culture groups containing HepLPC derived from different donor sources were treated with the CFSE cell proliferation detection kit, and then loaded onto a flow cytometer for detection to investigate the inhibition of the co-culture group containing the HepLPC derived from each donor source on the proliferation of the spleen cells and the proliferation of the spleen cells in the corresponding positive control group, and the results were as shown in FIGS. 112 to 115. The inhibition rates of the HepLPCs derived from the donor 1, the donor 2, the donor 3 and the donor 4 on proliferation of spleen cells were 91%, 93%, 85% and 89%, respectively. It could be seen that the inhibitory effects of the HepLPCs derived from different donor sources on proliferation of the spleen cells were different, which is conducive to the subsequent research on the heterogeneity of immune-resistant drugs.

### Examples 4-5

In this example, four donor-derived HepLPCs obtained in Example 2 were co-cultured with human peripheral blood mononuclear cells (PBMCs) respectively, and the proliferation of T cells in the PBMCs was stimulated with phytohemagglutinin PHA to investigate the inhibitory effect of the HepLPC on proliferation of the PBMCs.

Firstly, the peripheral blood of a healthy man was taken, diluted with an appropriate amount of D-PBS and Histopaque-1077, and then centrifuged at 2,000 rpm for 25 minutes. The white membrane layer in the middle was pipetted, added with D-PBS, and repeatedly centrifuged and washed, and the supernatant was discarded to obtain a cell precipitate. The cell precipitate was resuspended with a RPMI-1640 medium containing 10 g/ml of PHA, 10% PBS and 2 mM GlutaMAX to obtain a PBMC suspension.

The co-culture process of this example was as follows: the HepLPCs obtained after subculture and digestion of Example 4-1 were taken, and resuspended into a single-cell suspension with PBS; the single-cell suspension was washed with PBS repeatedly to ensure the removal of the digestion solution; the cells were resuspended with the RPMI-1640 complete medium containing 10% fetal bovine serum and the cell concentration was adjusted; the HepLPCs resuspended in the RPMI-1640 complete medium containing 10% fetal bovine serum were inoculated into a 6-well plate, and added with a PBMC suspension. By controlling the usage amount of the PBMC suspension and the HepLPC suspension and the concentration of each suspension, the number ratio of the HepLPC to the PBMC was 1:5, and the final concentration of PHA was 5 g/ml. Moreover, the PBMC culture group without addition of the HepLPC suspension was used as the positive control group.

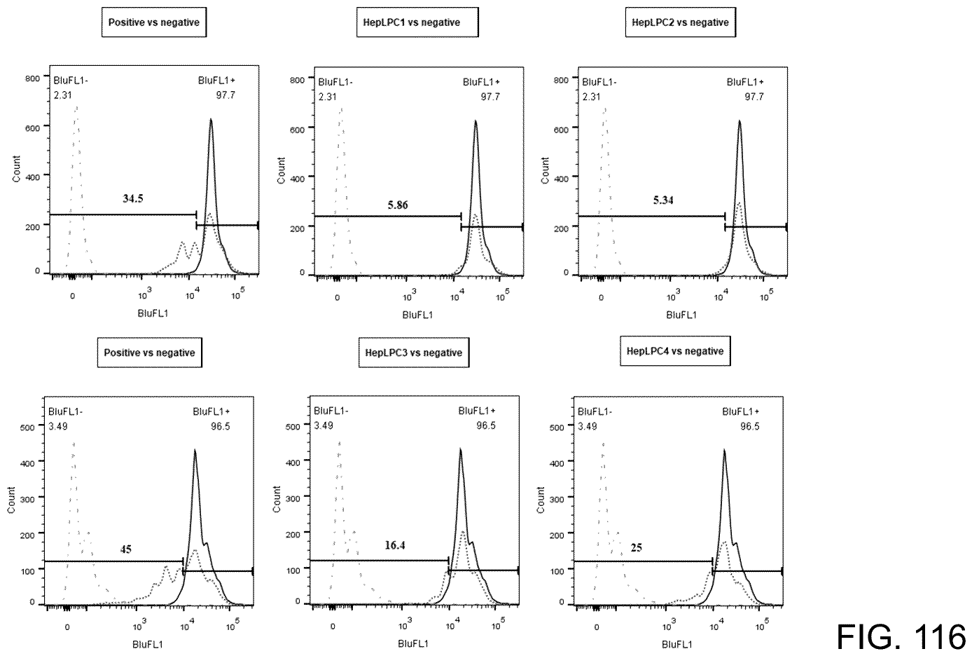

In this example, after 72 hours of co-culture, the positive control group and PBMC co-culture groups containing HepLPC derived from different donor sources were treated with the CFSE cell proliferation detection kit, and then loaded onto a flow cytometer for detection to investigate the inhibition of the PBMC co-culture group containing the HepLPC derived from each donor source on the proliferation of the PBMCs and the proliferation of the PBMCs in the corresponding positive control group, and the results were as shown in FIG. 116. It could be seen from the proportions of proliferated PBMCs in the positive control group and each co-culture group as shown in FIG. 116 that, the inhibition rates of the HepLPCs derived from a donor 1, a donor 2, a donor 3 and a donor 4 on the proliferation of the PBMCs were 85%, 83%, 64% and 44%, respectively. It could be seen that the inhibitory effects of the HepLPCs derived from different donor sources on proliferation of the PBMCs were different, which is conducive to the subsequent research on the heterogeneity of immune-resistant drugs.

### Examples 4-6

In this example, the HepLPC of Example 1 was used for cell transplantation, so as to investigate the effect of the HepLPC on the mouse autoimmune hepatitis model induced by ConA and mediated by T cells and NKT cells.

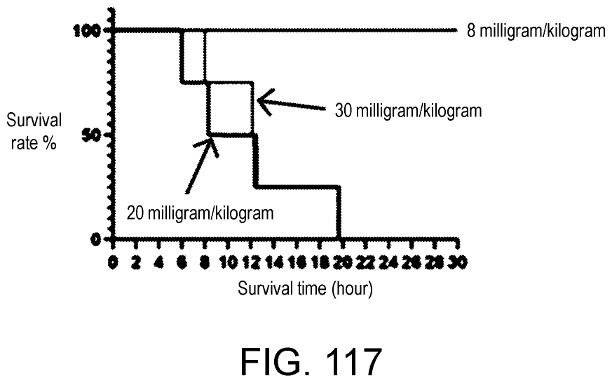

Firstly, C57BL/6 male mice of 6 weeks old and weighing 20-30 g in different groups (8 mice in each group) were injected with a ConA resuspended in PBS through tail vein injection according to injection doses of 8 milligrams/kilogram, 20 milligrams/kilogram and 30 milligrams/kilogram, to investigate the effect of the ConA concentration on the survival of the mice, so as to obtain a curve of the relationship between different injection doses of ConA and the survival rate of the mice as shown in FIG. 117. It could be seen from FIG. 117 that the lethal dose of ConA was not lower than 20 milligrams/kilogram, while 8 milligrams/kilogram was a non-lethal dose.

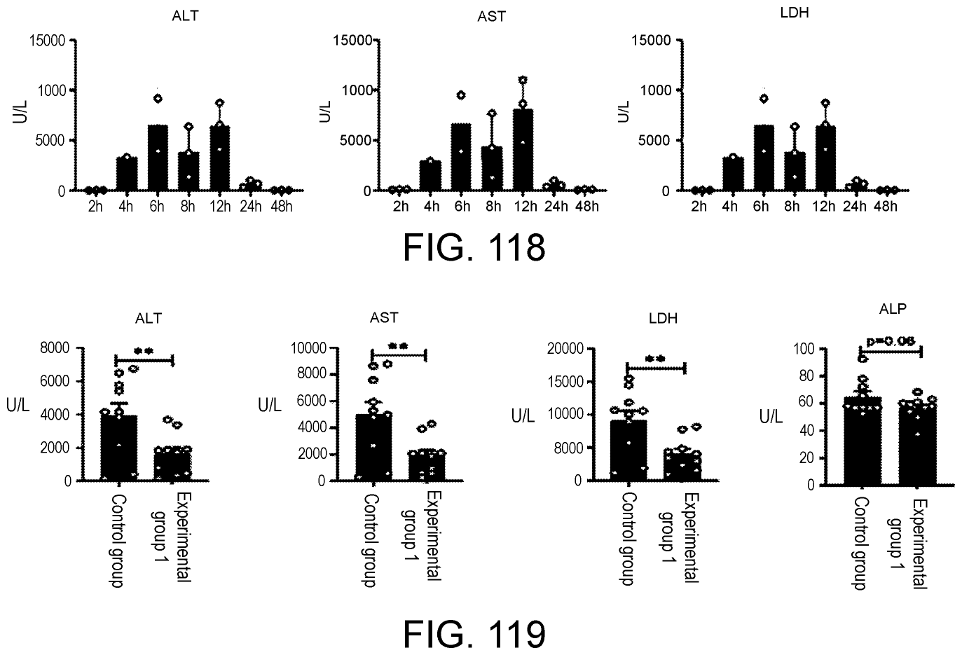

In this example, blood was taken from the orbits of the mice in the group injected with a dose of 8 milligrams/kilogram at different times, and the indicators ALT, AST and LDH of each group were investigated through blood biochemical level analysis, so as to obtain a comparison chart of levels of the indicators ALT, AST, LDH as shown in FIG. 118. Referring to FIG. 118, for the mice treated with a non-lethal dose of ConA, the blood ALT, AST, and LDH had the highest blood transaminases 6 h after injection. Therefore, the mice injected with a dose of 8 milligrams/kilogram through tail vein injection were successfully modeled at 6 hours after the end of the injection. 8 mice in the control group NC of this example were injected with the equal dose of PBS through tail vein injection.

In this example, the HepLPCs obtained after subculture and digestion in Example 4-1 were taken, and repeatedly resuspended ABD washed with PBS to ensure the purification effect, and then the HepLPCs were resuspended in PBS and grouped as follows:

Experimental group 1: 8 C57BL/6 male mice were injected with a HepLPC suspension resuspended in BS and ConA resuspended in PBS respectively through tail vein injection, the injection dose of HepLPC was controlled to be 10⁶/mouse, and the injection dose of ConA was a non-lethal dose of 8 milligrams/kilogram.

Experimental group 2: the difference of it from the experimental group 1 was that the injection dose of ConA was a lethal dose of 20 milligrams/kilogram.

Control group: each C57BL/6 male mouse was injected with the equal volume of PBS through tail vein injection.

The mice in each group survived 6 hours after completion of injection.

In this example, 6 hours after completion of injection, blood was taken from the orbits of the mice in the experimental group 1 and the control group, and the indicators ALT, AST, LDH and ALP of each group were investigated through blood biochemical level analysis, so as to obtain a comparison chart of the level of each indicator as shown in FIG. 119. The HepLPC transplantation significantly improved the levels of ALT, AST and LDH in mice.

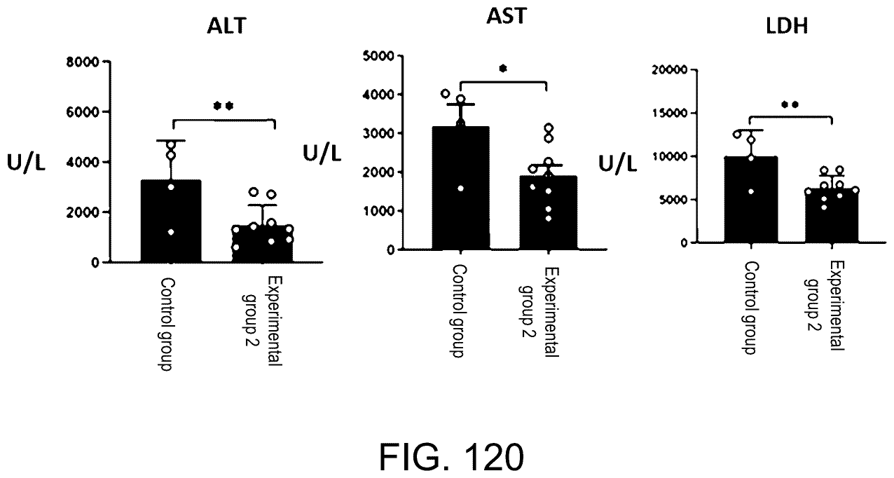

In this example, 6 hours after completion of injection, blood was taken from the orbits of the mice in the experimental group 2 and the control group, and the indicators ALT, AST and LDH of each group were investigated through blood biochemical level analysis, so as to obtain a comparison chart of the level of each indicator as shown in FIG. 120. Referring to FIG. 120, although the injection dose of ConA was a lethal dose, the transplantation of HepLPC also significantly improved the levels of ALT, AST and LDH in the mice.

What is claimed is:

1. A liver disease regulatory formulation, comprising a secretory supernatant of a hepatocyte-derived liver progenitor cell.

2. The liver disease regulatory formulation according to claim 1, wherein the secretory supernatant of the hepatocyte-derived liver progenitor cell comprises at least one miRNA which is at least one of miRNA-182, miRNA-183 and miRNA-574 and can effectively promote liver cell proliferation.

3. The liver disease regulatory formulation according to claim 1, wherein the secretory supernatant of the hepatocyte-derived liver progenitor cell comprises an active ingredient acting on a JAK-STAT pathway to inhibit activation of a hepatic stellate cell or induce death of the hepatic stellate cell.

4. The liver disease regulatory formulation according to claim 3, wherein the secretory supernatant of the hepatocyte-derived liver progenitor cell comprises at least one of a leukemia inhibitory factor, an endothelin, a colony stimulating factor, an amphiregulin and a fibroblast growth factor.

5. The liver disease regulatory formulation according to claim 1, wherein the secretory supernatant of the hepatocyte-derived liver progenitor cell comprises at least one of an inhibitory ingredient and a promoting ingredient, the inhibitory ingredient is used for inhibiting inflammatory activation of a type M1 macrophage, and the promoting ingredient is used for promoting production of a type M2 macrophage.

6. The liver disease regulatory formulation according to claim 1, wherein the secretory supernatant of the hepatocyte-derived liver progenitor cell comprises a secretory ingredient capable of inducing a receptor to establish effective immune tolerance by inhibiting proliferation of an immune cell.

7. The liver disease regulatory formulation according to claim 6, wherein the immune cell is any one of a macrophage, a B cell, a T cell, an NK cell and an NKT cell.

8. The liver disease regulatory formulation according to claim 1, wherein the secretory supernatant of the hepatocyte-derived liver progenitor cell is acquired from a culture product obtained by culturing the hepatocyte-derived liver progenitor cell in an in vitro medium.

9. The liver disease regulatory formulation according to claim 8, wherein the in vitro medium comprises a basal medium which is at least one of a HepX Basal medium, a DMEM/F12 cell culture medium, a William's E cell culture medium, a Neurobasal Medium cell culture medium, an MEM cell culture medium, a DMEM cell culture medium, a 1640RPMI cell culture medium and a F12 cell culture medium.

10. The liver disease regulatory formulation according to claim 9, wherein the in vitro medium further comprises at least one of a serum-like substance and a double antibody, the content of the serum-like substance does not exceed 20%, and the content of the double antibody does not exceed 20% based on a volume content of the basal medium.

11. The liver disease regulatory formulation according to claim 1, wherein the secretory supernatant of the hepatocyte-derived liver progenitor cell is extracted from a culture product obtained by: culturing a human hepatocyte in vitro with a medium containing a serum-like substance, which consists of a medium without the serum-like substance and the serum-like substance that accounts for 1-20% by volume of the medium containing the serum-like substance, until the confluence is not lower than 90%, then replacing the medium containing the serum-like substance with the medium without the serum-like substance, and continuing to conduct in vitro culture; and
the medium without the serum-like substance comprises a basal medium, a serum-free additive, a growth factor, a TGF-β signal inhibitor, a Wnt signaling pathway agonist and a ROCK kinase inhibitor.

12. A method for preparing the liver disease regulatory formulation according to claim 11, wherein the medium without the serum-like substance further comprises at least one of N-acetyl-L-cysteine and ascorbic acid.

13. A method for preparing the liver disease regulatory formulation according to claim 11, wherein the human hepatocyte is any one of a human primary hepatocyte, a human liver precursor cell and a human liver precursor-like cell.

14. A method for preparing the liver disease regulatory formulation according to claim 11, wherein based on the content in the medium without the serum-like substance:
a content of the growth factor is 0.1-100 nanograms/milliliter, a content of the ROCK kinase inhibitor is 0.1-100 micromoles/liter, a content of the Wnt signaling pathway agonist is 0.1-50 micromoles/liter, a content of the TGF-β signal inhibitor is 0.1-100 micromoles/liter, a content of the serum-like substance is no more than 20%, and a volume content of the serum-free additive is no more than 2%.

15. In vitro use of the liver disease regulatory formulation according to claim 1, comprising co-culturing the liver disease regulatory formulation with any one of a primary hepatocyte, a hepatic stellate cell, a macrophage and an immune-related cell.

16. The in vitro use of the liver disease regulatory formulation according to claim 15, wherein the step of co-culturing the liver disease regulatory formulation with the hepatic stellate cell comprises conducting the co-culture by using a co-culture medium, and a content of the liver disease regulatory formulation is controlled to be no less than 1% based on a volume percentage of the co-culture medium.

17. The in vitro use of the liver disease regulatory formulation according to claim 15, wherein the co-culture medium comprises an activator of the hepatic stellate cell.

18. The in vitro use of the liver disease regulatory formulation according to claim 15, wherein the step of co-culturing the liver disease regulatory formulation with the macrophage comprises co-culturing the liver disease regulatory formulation with a hepatic macrophage model which is an inflammatory cell model or a repair cell model.

19. The in vitro use of the liver disease regulatory formulation according to claim 15, wherein the step of co-culturing the liver disease regulatory formulation with the immune-related cell comprises inducing proliferation of the immune-related cell by using a stimulator.

20. The in vitro use of the liver disease regulatory formulation according to claim 19, wherein the immune-related cell is any one of a peripheral blood mononuclear cell and a spleen cell.

21. The in vitro use of the liver disease regulatory formulation according to claim 19, wherein the step of co-culturing the liver disease regulatory formulation with the immune-related cell comprises resuspending the liver disease regulatory formulation by using the co-culture medium, and controlling a volume concentration of the liver disease regulatory formulation in the co-culture medium to be no less than 5%, so that an inhibition rate of the liver disease regulatory formulation on the proliferation of the immune-related cell is no less than 30%.

22. The in vitro use of the liver disease regulatory formulation according to claim 19, wherein the step of co-culturing the liver disease regulatory formulation with the immune-related cell comprises co-culturing different liver disease regulatory formulations with the immune-related cell, and a culture supernatant of the hepatocyte-derived liver progenitor cells contained in the different liver disease regulatory formulations is derived from different donors.

23. Use of the liver disease regulatory formulation according to claim 1 in treatment of a liver disease, comprising investigating an effect on liver regeneration after an in vivo animal model is intervened with the liver disease regulatory formulation.

24. The use of the liver disease regulatory formulation in treatment of a liver disease according to claim 23, wherein the in vivo animal model is any one of a carbon tetrachloride-induced mouse acute liver failure model, an acetaminophen-induced mouse acute liver failure model, a thioacetamide-induced mammalian liver cirrhosis model, a carbon tetrachloride-induced mammalian liver cirrhosis model, a mammalian nonalcoholic steatohepatitis model, a mouse autoimmune hepatitis model induced by ConA and mediated by a T cell and a NKT cell, a rat model of immune rejection after liver cell or liver tissue transplantation, and a post-liver transplantation acute host anti-graft reaction model.
